(19) United States
(12) Patent Application Publication
(10) Pub. No.: US 2008/0152641 A1
(43) Pub. Date: Jun. 26, 2008

(54) Inhibitors of Siderophore Biosynthesis in Fungi

(75) Inventors: Hubertus Haas, Oberau (AT); Markus Schrettl, Worgl (AT)

Correspondence Address: FOLEY AND LARDNER LLP;SUITE 500, 3000 K STREET NW, WASHINGTON, DC, 20007, US

(73) Assignee: Autria Wirtschaftsservice

(21) Appl. No.: 10/592,583

(22) PCT Filed: Mar. 11, 2005
(86) PCT No.: PCT/EP2005/002654

§ 371 Date: Sep. 12, 2006

Related U.S. Application Data
(60) Provisional application No. 60/552,369, filed on Mar. 12, 2004.

(30) Foreign Application Priority Data
Mar. 12, 2004 (CA) 2,460,873

(51) Int. Cl. A61K 39/395; C12Q 1/02; C12Q 1/25; C12Q 1/68; A61K 31/70; C12Q 1/26; C12Q 1/48

(52) U.S. Cl. 424/130.1; 435/29; 435/4; 435/6; 435/25; 435/15; 514/44

(57) ABSTRACT

The present invention relates to methods for screening inhibitors of siderophore biosynthesis in fungi, preferably in Aspergillus species, particularly preferred in Aspergillus fumigatus comprising (a) contacting a cell expressing a fungal siderophore with a compound to be tested; (b) determining whether said cell is capable of siderophore biosynthesis in the presence of said compound to be tested when compared to a cell not contacted with said compound; and (c) identifying the compound which inhibits fungal siderophore biosynthesis. Accordingly, the invention also provides for a method for screening inhibitors of fungal siderophore biosynthesis comprising the steps of (a) contacting an enzyme involved in siderophore biosynthesis with a compound to be tested; (b) determining whether said enzyme is functional in the pathway of siderophore biosynthesis in the presence of said compounds to be tested when compared to an enzyme not contacted with said compound; and (c) identifying the compound which inhibits the enzymatic function involved in siderophore biosynthesis. In another aspect the present invention relates to a method of preparing a pharmaceutical composition for treating diseases associated with fungal infections, particularly, aspergillosis or coccidiosis comprising (a) identifying a compound which inhibits fungal siderophore biosynthesis; and (b) formulating said compound with a pharmaceutically acceptable carrier. In a further aspect, the present invention relates to a method for the production of a pharmaceutical composition comprising the steps of the aforementioned screening method and the subsequent step of mixing the compound identified to be an inhibitor of fungal siderophore biosynthesis with a pharmaceutically acceptable carrier. Moreover, the present invention envisages a pharmaceutical composition comprising an inhibitor of fungal siderophore biosynthesis as well as the use of such an inhibitor for the preparation of a pharmaceutical composition for the prevention and/or treatment of diseases associated with fungal infections, particularly, aspergillosis or coccidiosis.

The present invention relates to methods for screening inhibitors of siderophore biosynthesis in fungi, preferably in Aspergillus species, particularly preferred in Aspergillus fumigatus comprising (a) contacting a cell expressing a fungal siderophore with a compound to be tested; (b) determining whether said cell is capable of siderophore biosynthesis in the presence of said compound to be tested when compared to a cell not contacted with said compound; and (c) identifying the compound which inhibits fungal siderophore biosynthesis. Accordingly, the invention also provides for a method for screening inhibitors of fungal siderophore biosynthesis comprising the steps of (a) contacting an enzyme involved in siderophore biosynthesis with a compound to be tested; (b) determining whether said enzyme is functional in the pathway of siderophore biosynthesis in the presence of said compounds to be tested when compared to an enzyme not contacted with said compound; and (c) identifying the compound which inhibits the enzymatic function involved in siderophore biosynthesis. In another aspect the present invention relates to a method of preparing a pharmaceutical composition for treating diseases associated with fungal infections, particularly, aspergillosis or coccidiosis comprising (a) identifying a compound which inhibits fungal siderophore biosynthesis; and (b) formulating said compound with a pharmaceutically acceptable carrier. In a further aspect, the present invention relates to a method for the production of a pharmaceutical composition comprising the steps of the aforementioned screening method and the subsequent step of mixing the compound identified to be an inhibitor of fungal siderophore biosynthesis with a pharmaceutically acceptable carrier. Moreover, the present invention envisages a pharmaceutical composition comprising an inhibitor of fungal siderophore biosynthesis as well as the use of such an inhibitor for the preparation of a pharmaceutical composition for the prevention and/or treatment of diseases associated with fungal infections, particularly, aspergillosis or coccidiosis.

Most prokaryotes and all eukaryotes require iron for their growth. This transition metal has two readily available ionization states, ferrous and ferric iron, and thus is involved in a great variety of enzymatic processes including electron transfer in respiration, redox reactions carried out by numerous oxygenases and hydrogenases, and DNA-synthesis. While iron is one of the most abundant metals on earth, in aerobic environments it is present mostly in very insoluble compounds such as oxyhydroxide polymers. Consequently, the concentration of ferric iron in solution at neutral pH is probably not greater than 10-18 M (Neilands, J. Biol. Chem. 270 (1995), 26723-26726). On the other hand, an excess of iron within cells can be deleterious, because of the potential to catalyze the generation of cell damaging reactive oxygen species. Therefore, microbes have developed various highly regulated systems for iron uptake and storage. In the last decade, great advances have been made in the understanding of iron transport and intracellular distribution at the molecular level, especially in the baker's yeast Saccharomyces cerevisiae. This yeast certainly provides a useful paradigm of iron metabolism for other organisms. Due to a remarkable conservation of certain mechanisms involved in securing metal homeostasis between Saccharomyces and humans, studies of homologs of human disease genes in this yeast have shed light on the pathophysiology of several disorders (Askwith and Kaplan, Trends Biochem. Sci. 23 (1998), 135-138). However, an important difference exists between this best studied eukaryotic model microorganism and most other fungi—S. cerevisiae lacks the ability to synthesize siderophores although it can utilize siderophores produced by other species (Neilands, Comparative biochemistry of microbial iron assimilation. In: Winkelmann G., Winge D. R. (eds.) Iron Transport in microbes, plants and animals. Weinheim and VCH, New York (1987), pp. 3-34).

### Acquisition of Iron

As most species lack an excretory route for iron, the primary control point for iron homeostasis appears to be regulation of metal uptake across the plasma membrane. S. cerevisiae uses a variety of iron acquisition strategies, including separate high-affinity and multiple low-affinity uptake systems. This might also hold for other fungi and the explanation for such a diversity is probably that alternative mechanisms provide the organism with the ability to deal with a variety of environmental challenges. High-affinity systems are important in iron-limited conditions, whereas low-affinity systems play an important role when iron is more abundant. Furthermore, pathogenic fungi have potentially developed additional systems specialized to utilize host iron sources.

### High-Affinity Iron Uptake

Because iron is most commonly found as virtually insoluble ferric hydroxides, a general feature of high-affinity uptake systems is the necessity to solubilize ferric iron, whereby two major strategies have evolved in microorganisms: copper-dependent reductive iron uptake and copper-independent siderophore transport. The latter system is often termed “nonreductive iron assimilation”. However, it is important to note that nonreductive iron assimilation also contains a reductive step which occurs in contrast to reductive iron assimilation intracellularly subsequent to the uptake of iron. Various fungi utilize both strategies and siderophore uptake is also found in fungi unable to synthesize siderophores. Furthermore, siderophore-bound iron can in many cases be utilized by the reductive iron assimilatory pathway.

### Reductive Iron Assimilation

Reductive iron assimilation begins with solubilization of iron by extracellular reduction of ferric iron to ferrous iron which is subsequently taken up.

### Extracellular Reduction of Iron

Ferric iron is reduced to ferrous iron at the plasma membrane through transmembrane electron transfer mediated by the iron-regulated paralogous metalloreductases Fre1p, Fre2p, Fre3p, and Fre4p (Dancis, Proc. Natl. Acad. Sci. USA 89 (1992), 3869-3873); Georgatsou and Alexandraki, Mol. Cell. Biol. 14 (1994), 3065-3073; Yun, J. Biol. Chem. 276, (2001), 10218-10223). Fre1p and Fre2p have additionally been shown to facilitate copper uptake (Hassett and Kosman, J. Biol. Chem. 270 (1995), 128-134; Georgatsou, J. Biol. Chem. 272 (1997), 13786-13792) therefore the term metalloreductases is more appropriate than ferrireductases. Substrates for the reductive iron assimilatory system include iron salts, low-affinity iron chelates as ferric citrate, and siderophores like ferrioxamine B, ferrichrome, triacetylfusarinine C, enterobactin and rhodotorulic acid.

Evidence for membrane-bound reductive iron assimilatory systems has been obtained from studies of a broad array of fungi, including Schizosaccharomyces, Candida, Pichia, Hyphopichia, Kluyveromyces, Endomyces, Yarrowia, Cryptococcus, Ustilago, Histoplasma, Arxula, and Rhodotorula (Ecker and Emery, J. Bacteriol. 155 (1983), 616-622; Lesuisse, Anal. Biochem. 226 (1995), 375-377; Morrissey, Microbiology 142 (1996), 485-492; Askwith and Kaplan, J. Biol. Chem. 272 (1997), 401-405; Fedorovich, Biometals 12 (1999), 295-300; Nyhus and Jacobson, Infect. Immun. 67 (1999), 2357-2365; Timmerman and Woods, Infect. Immun. 67 (1999), 6403-6408). Homologs to S. cerevisiae metalloreductase-encoding genes have been detected in various fungi.

The reduced iron is subsequently taken up by low-affinity iron uptake systems active in iron-replete cells or the siderophore-independent high-affinity ferrous iron uptake system, which is expressed in iron-limited cells.

High-affinity ferrous iron uptake is best studied in S. cerevisiae. The combined action of the iron oxidase Fet3p and the permease Ftr1p might be required to import the specificity to the high-affinity transport of the potentially toxic metal iron. Low-affinity iron uptake is so far best studied in S. cerevisiae, too, yet, orthologues of the respective yeast gene FET4 are present in S. pombe, N. crassa and A. fumigatus.

Similarly to S. pombe, the genomes of both A. fumigatus and Neurospora crassa, but not Aspergillus nidulans contain loci with adjacent FET3 and FTR1 orthologous genes which are divergently transcribed in response to iron starvation. So far, it is not known if these two fungi have the capacity of reductive iron assimilation or if the Fet3p and Ftr1p homologs could alternatively be involved in extraction of iron from vacuolar stores, as it has been shown for the homologous S. cerevisiae Fet5p-Fth1p complex (Urbanowski and Piper, J. Biol. Chem. 274 (1999), 38061-38070).

### Nonreductive Iron Uptake (Siderophore Uptake)

Siderophore uptake involves the following steps: synthesis and excretion of an iron-free siderophore (desferrisiderophore), binding of iron by this chelator, import of the siderophore, and intracellular release of iron, probably by reduction. Subsequently, the iron-free siderophore or breakdown products are excreted. Furthermore, some siderophores appear to be not excreted, but synthesized exclusively for intracellular iron storage, e.g., ferricrocin in A. nidulans and N. crassa (Matzanke, J. Bacteriol. 169 (1987), 5873-5876; Oberegger, Mol. Microbiol. 41 (2001), 1077-1089).

There are numerous examples for fungi excreting more than one siderophore-type, possibly in order to adapt to different environmental conditions, e.g., Ustilago maydis excretes desferriferrichrome and desferriferrichrome A, whereby it utilizes ferrichrome A-bound iron exclusively via reductive iron assimilation and ferrichrome by uptake of the siderophore-iron complex (Ardon, J. Bacteriol. 180 (1998), 2021-2026). Various siderophore-producing fungi possess specific uptake systems for siderophore-types synthesized exclusively by other fungi, e.g., A. nidulans can take up various heterologous siderophores (xenosiderophores) including the hydroxamate-type siderophore ferrirubin synthesized by Aspergillus ochraceous and the catecholate-type siderophore enterobactin produced by various bacteria of the families Enterobacteriaceae and Streptomycetaceae (Fiedler, FEMS Microbiol. Lett. 196 (2001), 147-151; Oberegger (2001), loc. cit.). Such a strategy might have evolved for competitiveness with other organisms and/or conservation of metabolic energy. Some fungi are not able to synthesize siderophores, but nevertheless have the capacity to take up siderophores produced by other microorganisms, e.g., S. cerevisiae (Neilands (1987), loc. cit.; Lesuisse and Labbe, J. Gen. Microbiol. 135 (1989), 257-263).

### Siderophore Biosynthesis

Under conditions of iron depletion, most fungi excrete low-molecular weight (Mᵣ<1500) ferric iron chelators, collectively called siderophores. With the exception of carboxylates produced by zygomycetes (e.g., rhizoferrin produced by various Mucorales), most fungal siderophores are hydroxamates (van der Helm and Winkelmann, Hydroxamates and polycarbonates as iron transport agents (siderophores) in fungi. In: Winkelman G., Winge D. R. (eds.): “Metal ions in fungi”, New York, N.Y.: Marcel Decker, Inc. (1994), pp 39-148)). The nomenclature of siderophores is not uniform; in most cases they are named on the basis of their iron-charged forms, while the deferrated form is called de(s) ferrisiderophore. Detailed description of the chemistry of hydroxamates has been presented in van der Helm and Winkelmann, loc. cit. (1994). There are four major families of fungal hydroxamate-type siderophores for which representative structures and characteristics are known, i.e. rhodotorulic acid, fusarinines, coprogens, and ferrichromes. In all these fungal siderophores, the nitrogen of the hydroxamate group is derived from N⁵-hydroxyornithine. Completion of the hydroxamate prosthetic group requires acylation with the simplest group being acetyl and more complex groups being anhydromevalonyl or methylglutaconyl. Most siderophores contain three covalently linked hydroxamates in order to form an octahedral complex. The link between the hydroxamate groups can be peptide bonds or ester bonds. The simplest structure, rhodotorulic acid produced by the basidiomycetous yeast Rhodotorula, is a dipeptide built from two N⁵-acetyl-N⁵-hydroxyornithines linked head-to-head. The prototype of fusarinines, the cyclic fusarinine C (or fusigen), consists of three N⁵-cis-anhydromevalonyl-N⁵-hydroxyornithines (termed cis-fusarinine), linked by ester bonds. Fusarinine C is relatively labile; acetylation of the primary amino acid groups results in the more stable triacetylfusarinine C. Fusarinines are produced, e.g., by Fusarium spp. and Aspergillus spp. Coprogens contain two trans-fusarinine moieties connected by a peptide bond head-to-head to form a diketopiperazine unit (dimerium acid) and a third trans-fusarinine molecule esterified to the C-terminal group of dimerium acid. Coprogens are produced by, e.g., Fusarium dimerium, Neurospora crassa, and Histoplasma capsulatum. Ferrichromes are cyclic hexapeptides consisting of three N⁵-acyl-N⁵-hydroxyornithines and three amino acids—combinations of glycine, serine or alanine. Ferrichromes are produced, e.g., by the basidiomycete U. maydis and the ascomycetes Aspergillus spp. and N. crassa. It is important to note that “ferrichrome” and “coprogen” refer to specific members of their respective family.

The first committed step in siderophore biosynthesis is the N⁵-hydroxylation of ornithin catalyzed by ornithine N⁵-oxygenase, also termed ornithine N⁵-hydroxylase, and requires O₂, FAD and NADPH. The first characterized fungal ornithine N⁵-oxygenase-encoding gene was sid1 of U. maydis (Mei, Proc. Natl. Acad. Sci. U.S.A. 90 (1993), 903-907). Sid1 reveals homology to E. coli lysine N⁶-hydroxylase, which catalyzes the first step in the biosynthesis of the bacterial siderophore aerobactin. Expression of sid1 is Urbs-mediated repressed by iron at the transcriptional level, and disruption of sid1 blocks synthesis of ferrichrome and ferrichrome A, the two siderophores produced by U. maydis (Voisard, Mol. Cell. Biol. 13 (1993), 7091-7100). Recently, identification of the A. nidulans sid1 orthologue, sidA, has been reported (Oberegger, Biochem. Soc. T. 30 (2002), 781-783). Expression of sidA is regulated by iron and this control is mediated by the A. nidulans Urbs1 orthologue SreA. As in Ustilago, disruption of the ornithine N⁵-oxygenase-encoding gene sidA leads to a block in synthesis of all siderophores in A. nidulans—fusarinine, triactylfusarinine and ferricrocin. sid1 orthologous genes are present in the genomes of the siderophore-producing fungi A. fumigatus, N. crassa, and Aureobasidium pullulans; consistently, the genome of the siderophore nonproducer S. cerevisiae lacks a homologous sequence. Noteworthy, Schizosaccharomyces pombe, suggested to lack siderophore biosynthesis (Neilands, J. Biol. Chem. (1995), 26723-26726), possesses a gene with striking similarity to fungal ornithine N⁵-oxygenase. Searches in the genome of C. albicans, for which siderophore biosynthesis was reported (Ismail, Biochem. Biophys. Res. Commun. 130 (1985), 885-891), failed to identify possible sid1 orthologues. Furthermore, several attempts to identify hydroxamate siderophores in C. albicans were unsuccessful. Therefore, it is questionable if this yeast is indeed able to synthesize hydroxamate-type siderophores.

The formation of the hydroxamate group is conducted by the transfer of an acyl group from acyl CoA derivatives to N⁵-hydroxyornithine. An N⁵-hydroxyornithine:acetyl CoA-N⁵-transacetylase was found in U. sphaerogena and Rhodotorula pilimanae (Ong and Emery, Arch. Biochem. Biophys. 148 (1972), 77-83). Some siderophores require, in addition, acetylation at the N²-amino group of the hydroxamate, e.g., coprogen and triacetylfusarinine C. So far, no sequence information is available for these enzymes.

Completion of siderophore biosynthesis requires linking of the hydroxamate groups; in the case of ferrichromes, additional incorporation of three amino acids is needed. This task is carried out by nonribosomal peptide synthetases, similar to the synthesis of many peptide antibiotics. These synthetases are exceptionally large enzymes with a modular construction (Marahiel, Chem. Biol. 4 (1997), 561-567). Each module contains a substrate specific adenylation domain, a peptidyl carrier domain, and a condensation domain. As the acyl carrier domains of fatty acid and polyketide synthases, the peptidyl carrier domain contains phosphopantetheine as a covalently linked cofactor, which is attached by 4′-phosphopantetheine transferase. Recently, npgA of A. nidulans has been found to encode such an activity (Mootz, FEMS Microbiol. Lett. 213 (2002), 51-57). The genome sequences of A. fumigatus and N. crassa appear to contain only a single npgA orthologue. Consequently, only a single enzyme may be able to transfer the cofactor to a broad range of enzymes containing acyl and peptidyl carrier domains. Peptide synthetases are able to form peptide and ester bonds, the peptidyl chain grows directionally in incremental steps, and for cyclic products, the final condensation must lead to ring closure. The only functional characterized fungal peptide synthetase-encoding gene involved in siderophore biosynthesis is sid2 of U. maydis (Yuan, J. Bacteriol. 183 (2001), 4040-4051). As with many microbial genes involved in the same biosynthetic pathway, sid2 and sid1 are clustered: these two genes are divergently transcribed from a 3.7-kb intergenic region and show the same expression pattern. Disruption of sid2 leads to a block of ferrichrome biosynthesis, whereas the synthesis of the structurally different ferrichrome A is unaffected. sid2 encodes a protein, 3947 amino acids in length, which contains three similar modules of approximately 1000 amino acids plus an additional peptidyl carrier domain. This suggests that Sid2 might be able to synthesize a tripeptide. However, it was hypothesized that this enzyme might be responsible for formation of the complete hexapeptide via repeated use of one or more modules. A peptide synthetase (Psy1) said to be involved in synthesis of dimerium acid in Trichoderma virens (Wilhite, Appl. Environ. Microbiol. 67 (2001), 5055-5062) was subsequently shown to participate rather in formation of the 18-amino acid peptide peptaibol (Wiest, J. Biol. Chem. 277 (2002), 20862-20868). Peptide synthetase-encoding genes are present in the genomes of most fungi, but these are not necessarily involved in siderophore biosynthesis because most fungi produce numerous peptidic secondary metabolites and exact prediction of the synthesized product from the primary sequence of the peptide synthetase is impossible. Nevertheless, there are further candidates for fungal genes encoding siderophore peptide synthetase, e.g., sidB and side from A. nidulans, which are regulated by the iron-responsive repressor SREA (Oberegger, loc. cit. (2002)). Furthermore, in S. pombe and A. pullulans, peptide synthetase-encoding genes are found to be clustered with sid1 homologs, which might be indicative of involvement in a common pathway. In the respective A. pullulans gene cluster, an ATB-binding cassette (ABC) transporter is additionally present. ABC-transporters are transmembrane proteins which couple the energy of ATP hydrolysis to the selective transfer of substrates across biological membranes (Higgins, Cell 82 (1995), 693-696. Many ABC transporters are known as multidrug resistance (MDR) transporters due to involvement in export of toxic molecules from the cell. Members of this protein family might also be involved in intracellular transmembrane trafficking of siderophore precursors or excretion of siderophores. In A. nidulans, the expression of the ABC-transporter AtrH is repressed SREA-dependently by iron, suggesting that this transporter might be involved in iron metabolism (Oberegger, loc. cit. (2002)). Subsequent to synthesis and excretion of the siderophores, these chelators solubilize extracellular ferric iron. The binding constant for iron of siderophores containing three bidentate ligands is 10³⁰ M⁻¹, or greater, allowing microbes to extract iron even from stainless steel (Neilands, loc. cit. (1995); Askwith, Mol. Microbiol. 20 (1996), 27-34). The iron of the siderophore-iron complex is then utilized either by the reductive iron assimilatory system, or the whole siderophore-iron chelate is taken up by specific transport systems.

### Siderophore Uptake and Utilization

The high-affinity nonreductive iron assimilation system is specialized for the uptake of siderophore-bound iron. In S. cerevisiae, siderophore uptake depends on four members of the family 16, previously designated UMF (unknown major facilitator) and newly designated SIT (siderophore-iron transporter) family of the major facilitator superfamily (Pao, Microbiol. Mol. Biol. Rev. 62 (1998), 1-34; Winkelmann, Siderophore transport in fungi. In: Winkelman G. (ed.): “Microbial transport systems.” Weinheim: Wiley-VCM (2001)).

The acquisition of iron is recognized as a key step in the infection process of any pathogen, since this metal is tightly sequestered by high-affinity iron-binding proteins in mammalian hosts, e.g., transferrin, lactoferrin, ferritin and hemoglobin (Weinberg, J. Eukaryot. Microbiol. 46 (1999), 231-238). Furthermore, hosts have developed an elaborate iron withholding defense system (Weinberg, Perspect. Biol. Med. 36 (1993), 215-221). In bacteria, two systems have been developed to acquire iron from their hosts. These include binding and uptake of host iron compounds, e.g., heme or transferrin, and capture of iron from host proteins via siderophore biosynthesis and uptake (Clarke, Curr. Top. Med. Chem. 1 (2001), 17-30). There are numerous examples of fungi whose viability in culture or in hosts are enhanced by iron and/or suppressed by iron chelators reviews dealing with the impact of iron in fungal infectious diseases have recently been published (Howard, Clin. Microbiol. Rev. 12 (1999), 394-404; Weinberg, loc. cit. (1999)). In contrast to bacteria (Ratledge and Dover, Annu. Rev. Microbiol. 54 (2000), 881-941; Crosa and Walsh, Microbiol. Mol. Biol. Rev. 66 (2002), 223-249), proofs for a direct relation of fungal iron acquisition systems and virulence are scarce, probably due to a delay in development of molecular tools for manipulation of pathogenic fungi.

U. maydis mutants deficient in siderophore biosynthesis have unchanged virulence in plants (Mei, loc. cit. (1993)) which might have two reasons: U. maydis possesses other high-affinity iron uptake systems able to complement this defect—in this respect it is important to note that reductive iron assimilation has been shown in this fungus (Ardon, loc. cit. (1998))—or only a small subset of plant cells display low iron availability as recently suggested (Joyner and Lindow, Microbiology 146 (2000), 2435-2445).

For zoo-pathogenic fungi, it has been shown that reductive iron assimilation constitutes a virulence factor: CaFtr1p-deficient C. albicans mutants are unable to establish systemic infection in mice (Ramanan and Wang, Science 288 (2000), 1062-1064). However, differences were found in the pathogenicity of various mutants: virulence of C. albicans deficient in CaFet3p, assumed to be as essential as CaFr1p for reductive iron assimilation, is unaffected (Eck, Microbiology 145 (1999), 2415-2422). Moreover, deficiency in CaCcc2p, supposed to be necessary for copper loading of CaFet3p, does not lead to reduced virulence (Weissman, loc. cit. (2002)). These differences could be explained by differences in experimental conditions, such as mouse strains or fungal culture conditions before inoculation—which has been shown to possibly affect virulence (Odds, Microbiology 146 (2000), 1881-1889). Alternatively, unlike the situation in S. cerevisiae, CaFtr1 might function independently of CaFet3p in C. albicans.

For several bacterial species the essential role of siderophores in the pathogenicity has unequivocally been established (Ratledge and Dover, loc. cit. (2000)). Numerous pathogenic fungi produce siderophores, e.g., A. fumigatus, H. capsulatum, Sporotrix schenckii, Microsporum spp., Blastomyces dermatitis, and Trichophyton spp. (Burt, Infect. Immun. 35 (1982), 990-996; Holzberg and Artis, Infect. Immun. 40 (1983), 1134-1139; Nilius and Farmer, J. Med. Vet. Mycol. 28 (1990), 395-403; Howard, Clin. Microbiol. Rev. 12 (1999), 394-404), but the role of siderophore production in fungal virulence has not been clarified yet. Remarkably, the Candida siderophore transporter CaArn1p/CaSit1p is required for a specific process of infection, namely epithelial invasion and penetration, while it is not essential for systemic infection by C. albicans (Heymann, Infect. Immun. 70 (2002), 5246-5255; Hu, J. Biol. Chem. 277 (2002), 30598-30605). In case the siderophore system proves to be important for pathogenicity of various fungi, it might represent an attractive new target for an antifungal chemotherapy because the underlying biochemical pathways are absent in human cells. Moreover, it has been shown that drug-siderophore conjugates have great potential for species-selective delivery of antimicrobials to populations of microorganisms (Roosenberg, Curr. Mol. Chem. 7 (2000), 159-197). The studies of C. albicans mutants deficient in both siderophore uptake and reductive iron assimilation revealed the existence of an additional independent mechanism of iron uptake from host tissues in this yeast: uptake of hemin and hemoglobin (Heymann, loc. cit. (2002); Weissman, loc. cit. (2002)). The respective receptors have not been identified yet.

Important to note, siderophores may not only be important in fungal pathogenicity, but can also be beneficial to other organisms. Mycorrhizal symbiosis is a common phenomenon in all terrestrial plant communities. It is well documented that mycorrhizal infection affects the mineral nutrition of the plant, including micronutrient uptake (Perotto and Bonfante, Trends Microbiol. 5 (1997), 496-504). It was shown that a number of mycorrhizal fungi produce hydroxamate-type siderophores and, therefore, fungal siderophore production potentially contributes to the iron supply of plants (Haselwandter, Crit. Rev. Biotechnol. 15 (1995), 287-291; Haselwandter and Winkelmann, Biometals 15 (2002), 73-77). Moreover, fungal siderophores might indirectly improve the iron status of plants because iron solubilized by hydrolysis products of fungal siderophores present in the soil, e.g., fusarinines and dimerium acid, is an excellent source for iron nutrition of plants (Hordt, Biometals 13 (2000), 37-46). Furthermore, it has to be noted that a siderophore from Streptomyces spp., desferrioxamine (desferal), continues to be the best treatment for iron overload diseases in humans, especially thalassemy (Richardson and Ponka, Am. J. Hematol. 58 (1998), 299-305). Unfortunately, desferal therapy suffers from not being orally effective. Fundamental studies on the molecular biology of fungal siderophore biosynthesis might provide genes which can be engineered to create novel chelators for clinical use.

The genus Aspergillus is one of the most ubiquitous microorganisms worldwide and various Aspergillus species are responsible for the clinical syndromes of allergic bronchopulmonary aspergillosis, aspergilloma and pulmonary aspergillosis. In mammalian hosts iron is tightly sequestered by high-affinity iron-binding proteins, and therefore microbes require efficient iron-scavenging systems to survive and proliferate within the host. Under iron starvation, most fungi synthesize and excrete low-molecular-weight, iron specific chelators—called siderophores—which have therefore often been suggested to function as virulence factors.

Among the genus Aspergillus, Aspergillus fumigatus has become the most important airborne fungal pathogen of humans. Clinical manifestations are ranging from allergic to invasive disease, largely depending on the status of the host's immune system. Colonization with restricted invasiveness can occur in the immunocompetent host, disseminated infections are observed in immunocompromised patients. Invasive aspergillosis increased dramatically in incidence during the last decades with advances in transplantation medicine and the therapy of hematological disorders. It is associated with a mortality rate of 30-98% reflecting that the possibilities of therapeutic intervention are very limited (Denning, Clin. Infect. Dis. 26 (1998), 781-803; Latge, Clin. Microbiol. Rev. 12 (1999), 310-350). A. fumigatus, which accounts for approximately 90% of aspergillosis, is a typical saprophytic fungus found in almost all sorts of decaying organic material, e.g. compost. It is still a matter of debate if this fungus has specific pathogenicity factors (Latge, loc. cit. (1999)). Inactivation of metabolic genes, which cause auxotrophies, impair pathogenicity in a mouse model. However, none of the other genes analyzed so far—including genes encoding proteases, a ribonuclease, or a polyketide synthase involved in pigment synthesis—led to a complete loss of virulence. These data support the hypothesis that pathogenesis by A. fumigatus is a multifactoral process. Most likely, A. fumigatus possesses a combination of physiological features to cope with the immune system and to acquire essential nutrients. One of the most important nutrients in the infection process of any pathogen is iron because this metal is an essential cofactor of enzymes in many biological processes including DNA replication and electron transport. Moreover, mammals posses an elaborate iron-withholding defense system against microbial infections (Weinberg, J. Eukaryot. Microbiol. 46 (1999), 231-268). Fungi have developed various high-affinity mechanism of iron acquisition (Van Ho, Annu. Rev. Microbiol. 56 (2002), 237-261; Haas, loc. cit. (2003); Leong and Winkelmann, Met. Ions. Biol. Syst. 35 (1998), 147-186; Kossmann, Mol. Microbiol. 47 (2003), 1185-1197)), including (i) solubilization of iron by enzymatic reduction of ferric iron and subsequent uptake of ferrous iron by a complex consisting of a ferroxidase and a coupled high affinity ferric permease (ii) uptake of heme-iron, and (iii) mobilization of iron by siderophores.

For over four decades, the principal target of antifungal therapy has been the fungal cell membrane sterol ergosterol. Although this has proven to be a successful and relatively selective antifungal target, collateral toxicity to mammalian cells (amphotericin B) and drug interactions (azoles) have been by-products of agents that target the fungal cell membrane (Tkacz and DiDomenico, Curr. Opin. Microbiol. 4 (2001), 540-545). These limitations, together with the problem of development of resistance against these treatments prompts the development of new antifungal compounds (Canuto and Rodero, Lancet Infect. Dis. 2 (2002), 550-563). Recently, beta(1,3)-glucan synthase inhibitors (echinocandins) have been introduced but need to be investigated further in proper trials (Girmenia and Martino, Curr. Opin. Oncol. (2003), 283-288). As mentioned hereinabove, Aspergillus species, in particular Aspergillus fumigatus causes more infections worldwide than any other mould. Four percent of all patients dying in tertiary care hospitals in Europe have invasive aspergillosis. The fungus causes allergic diseases in asthmatics and patients suffering from cystic fibrosis. Invasive aspergillosis can occur in individuals with cavities caused by tuberculosis or other cystic lung diseases. In view of the fact that none of the so far analyzed genes of Aspergillus fumigatus led to a complete loss of pathogenicity, there is a demand for new drugs which led to a complete loss of pathogenicity.

Thus, the technical problem of the present invention is to comply with the needs described above. The solution to this technical problem is achieved by providing the embodiments characterized in the claims.

Accordingly, in one aspect the present invention relates to a method for screening inhibitors of fungal siderophore biosynthesis comprising
(a) contacting a cell expressing a fungal siderophore with a compound to be tested;
(b) determining whether said cell is capable of siderophore biosynthesis in the presence of said compound to be tested when compared to a cell not contacted with said compound; and
(c) identifying the compound which inhibits fungal siderophore biosynthesis.

It was surprisingly found that siderophore biosynthesis is essential for virulence of fungi, in particular for virulence of Aspergillus species, more particularly for virulence of Aspergillus fumigatus. In particular, it is demonstrated in the appended Examples hereinbelow that each of the novel Aspergillus fumigatus sidA (Af sidA), at1 (Af-at1), at 2 (Af-at2) and sidD (Af-sidD) genes provided herein is not essential for survival of A. fumigatus in standard growth media, but is essential for full virulence of A. fumigatus. The gene sidA encodes an oxygenase. Northern analysis demonstrated that the expression of at1 (encoding a transacylase), sidD (encoding a nonribosmal peptide synthetase) and at2 (encoding a transacylase) was—similar to sidA (FIG. 2)—found to be induced during iron depleted conditions (FIG. 9), which suggested involvement of the gene products in iron metabolism of A. fumigatus. The term “virulence” when used in the context of the present invention means capacity of a microorganism, preferably of a fungal species, more preferably of an Aspergillus spec. and most preferably of Aspergillus fumigatus to cause disease. Yet, the term “pathogenicity” when used in the present application means the ability of a microorganism, preferably of a fungal species, more preferably of an Aspergillus spec. and most preferably of Aspergillus fumigatus to inflict damage, e.g. diseases caused by Aspergillus spec. as described hereinbelow on the host.

In the development of new drugs, an important step is the validation of a drug target. Target validation encompasses the proof for the essential nature of a target and the capacity for selective inhibition of that target in vivo. Selective toxicity may be achieved by taking advantage of unique features of the pathogen's metabolism. The essential nature of a target is usually demonstrated by the correlation of chemical or genetic reduction of target activity with the loss of pathogen growth. Ultimately, a target must be validated in vivo demonstrating loss of virulence of respective mutants. Validated targets are then exploited for high-throughput compound screening. Due to high experimental costs and ethical reasons, target validation procedures—similar to the classical screening for antifungal compounds—usually screen for essential genes only “in vitro” using different formulations of solid or liquid growth media, and not animal models like mice. A serious disadvantage of such procedures is the neglect of targets, which are essential only during the pathogenic phase but not during saprophytic growth.

As demonstrated by the present invention the Af-sidA, Af-at1, Af-at2, Af-rac1 or Af-sidD gene is not essential for survival of A. fumigatus on standard growth media used for screening and, therefore, Af-sidA or respective mutants would not have turned up in standard screenings for drug targets.

Remarkably, however, deletion of the Af-sidA completely abolished the capacity of A. fumigatus to establish systemic infection in a murine model. Furthermore, deletion of Af-at1, Af-at2 or Af-sidD significantly reduced the capacitiy of A. fumigatus to establish systemic infection in a murine model. The same phenotype is expected for a deletion of the Af-rac1 gene described herein. These data show for the first time unequivocally that the siderophore system plays a central role in the pathogenicity of a fungus. Accordingly, the genes and gene products provided herein are valuable drug targets. Even more remarkable is the finding of the application that loss of the Af-at2 gene product which is involved in the conversion of fusarinine C into triacetylfusarinine C leads to abolishment of establishing systemic injections of A. fumigatus in a mouse model. This is because already fusarinine C is a siderophore capable of iron-uptake. However, unexpectedly, triacetylfusarinine C, the extracellular siderophore of A. fumigatus, is crucial for the virulence of A. fumigatus. Though, in the Af-at2 deletion triacetylfusarinine C is not synthesized and, thus, the dramatically reduced virulence was observed in a mouse model for A. fumigatus systemic infections. Since the Af-at2 deletion mutant would not have been paid attention in standard assays screening for “in vitro” essential genes, because it does not display a phenotype, even on blood agar (see Example 26), it would not have been found as being important at all.

These reasons and the fact that mammals lack a similar system make the siderophore system an attractive target for development of therapies against Aspergillus spec., in particular Aspergillus fumigatus and most likely also other siderophore-producing fungi. In this respect it is important to note that numerous pathogenic fungi produce siderophores (Howard, Clin. Microbiol. Rev. 12 (1999), 394-404). L-ornithine-N⁵-monooxygenase (OMO) which is, for example, encoded by the Af-sidA gene, represents the first committed step of biosynthesis of hydroxamate-type siderophores as will be described in detail hereinbelow.

Consistently, data base searches (http://www.ncbi.nlm.nih.gov/blast/; http://www.ncbi.nlm.nih.gov/sutils/genom_tree.cgi) identified putative Af-sidA orthologs in the genomes of numerous fungi, including Aureobasidium pullulans, Neurospora crassa, Aspergillus nidulans, Aspergillus oryzae, Schizosaccharomyces pombe, U. maydis, Gibberella zeae, and Coccidioides posadasii which could also be used for screening of inhibition of siderophore biosynthesis. At1, at2, rac1 as well as sidD can also be found, inter alia, in A. oryzae and A. nidulans. Accordingly, also these organisms may be used in methods provided herein. The finding that the Af-sidA, Af-at1, Af-at2 or Af-sidD gene is essential for virulence of A. fumigatus is even more striking in view of the fact that A. fumigatus like other fungi having a reductive iron assimilation system, possesses a reductive iron assimilation system as is shown in the appended Examples hereinbelow which has been shown in said other fungi to be relevant for virulence or pathogenicity.

For example, siderophore biosynthesis-deficient mutants of the basidiomycete Ustilago maydis, which utilizes in addition a reductive iron assimilatory system (Ardon, J. Bacteriol. 180 (1998), 2021-2026), have unchanged virulence in plants (Mei, Proc. Natl. Acad. Sci. U.S.A. (1993), 903-907). Moreover, it was reported recently that the reductive iron assimilation system is essential for virulence of this plant pathogen (Eichhorn, VAAM-Tagung der Pilze, Göttingen, Germany (2003)).

In Candida albicans, which is able to utilize hydroxamate-type siderophores but unable to synthesize them itself (Haas, Appl. Microbiol. Biotechnol. 62 (2003), 316-330), the siderophore transporter CaArn1p/CaSit1p has been found to be required for epithelial invasion and penetration, while it is not essential for systemic infection (Heymann, Infect. Immun. 70 (2002), 5246-5255; Hu, J. Biol. Chem. 277 (2002), 30598-30605). In systemic infection by this yeast, the high-affinity iron permease CaFtr1, a component of the reductive iron assimilation system, has been shown to be essential (Ramanan and Wang, Science 288 (2000), 1062-1064).

A. nidulans was shown to employ only one high-affinity iron uptake strategy: siderophore-mediated iron uptake (Eisendle, Mol. Microbiol. 49 (2003), 359-375). In particular, in Eisendle (2003), loc. cit. it is described that deletion of the A. nidulans sidA gene leads to a complete loss of excreted and cellular siderophores and, thus, sidA-deficient strains were unable to grow, unless the growth-medium was supplemented with siderophores. This finding is, however, contrary to the finding of the present invention that deletion of the orthologue of the A. nidulans sidA, i.e. the A. fumigatus sidA gene does not lead to the incapability of saprophytic growth. Hence, A. nidulans appears to be an exception among fungi: in contrast to this model ascomycete, all other analyzed fungal species analyzed so far have shown to utilize reductive iron assimilation as described herein (e.g. Candida albicans, Schizosaccharomyces pombe, Ustilago maydis) or possess genes encoding putative components of this system (e.g. N. crassa, A. fumigatus, Gibberella zeae, Magnaporthe grisea, Cryptococcus neoformans, Coccidioides posadasii, Claviceps purpurea, Rhizopus oryzae, Pichia pastoris, Arzula adeninivorans) (http://www.nebi.nlm.nih.gov/blast/Blast.cgi). Moreover, some human-pathogenic fungi are unable to synthesize hydroxamate-type siderophores, e.g. Candida albicans and Cryptococcus neoformans, ruling out the possibility of siderophores being a general fungal virulence factor (Haas, Appl. Microbiol. Biotechnol. 62 (2003), 311-330; Jacobsen, Infect. Immun. 66 (1998), 4169-4175). From the above, it could be expected that as long as a fungus has the capacity for reductive iron assimilation it is pathogenic. Furthermore, A. nidulans which is closely related to A. fumigatus has apparently only one system for iron uptake whereby deficiency of “committed step enzyme ornithine monooxygenase” leads to loss of growth. It thus follows that it could once more be not expected that the Af-sidA gene, loss of which has no apparent phenotype, plays an essential role in virulence and/or pathogenicity.

Furthermore, in contrast to A. nidulans, the genome of A. fumigatus contains genes encoding putative components of a second high-affinity iron uptake system (reductive iron assimilation: orthologs to S. cerevisiae Fet3p and Ftr1p, termed FetC and FrtA in A. fumigatus) plus a low-affinity iron permease (ortholog to S. cerevisiae Fet4p, termed FetD in A. fumigatus). The putative second high-affinity iron uptake system of A. fumigatus makes the finding that Af-sidA is essential for virulence once again even more surprising.

As is described in the appended Examples below, to elucidate the function of Af-sidA, a deletion mutant for this gene was constructed. In the generated mutant allele of ΔAf-sidA, the entire coding region, 279 bp downstream and 137 bp upstream region of the gene was replaced by the hygromycine resistance (hph) marker. Reversed-phase-HPLC analysis according to Oberegger (Mol. Microbiol. 41 (2001), 1077-1089) demonstrated that the L-ornithine-N⁵-monooxygenase-deficient ΔAf-sidA strain lost the ability to produce both triacetylfusarinine C and ferricrocin. Compared to the wildtype (wt), ΔAf-sidA showed a radial growth rate of 61% during iron-replete and 27% during iron depleted conditions. This feature distinguishes ΔAf-sidA from the respective A. nidulans mutant, which is not able to grow without siderophore supplementation (Eisendle, Mol. Microbiol. 49 (2003), 359-375), and indicates that A. fumigatus possesses in contrast to A. nidulans an alternative iron assimilation system sufficient to enable growth during these conditions. As opposed to A. nidulans, the A. fumigatus genome sequence contains one putative ferroxidase and one potential high-affinity iron permease encoding gene, suggesting that A. fumigatus utilizes in addition to the siderophore system reductive iron assimilation (Haas, Appl. Microbiol. Biotechnol. 62 (2003), 316-375). The reductive iron assimilatory system has been shown to be copper-dependent due to the copper-requirement of the ferroxidase (Askwith, Cell 28 (2003), 403-410). Consistent with reductive iron assimilation being responsible for the residual growth of ΔAf-sidA, the use of copper-deficient media or the addition of the copper-specific chelator bathocuproine disulfonic acid in a concentration of 30 μM decreased the radial growth rate of ΔAf-sidA 46% and 25% compared to the wt. The growth of ΔAf-sidA was increased to 84% and 99% by supplementation with 1.5 mM FeSO₄ and 10 μM ferricrocin, respectively. Taken together, these data demonstrate that A. fumigatus possesses at least one siderophore-independent iron uptake mechanism which is also shown in the appended Examples.

In particular, mutants deficient in both SidA and FtrA were unable to grow unless supplemented with siderophores or high concentrations of ferrous iron, revealing that SidA and FtrA are components of alternative high-affinity iron uptake mechanisms and the existence of an additional low-affinity iron uptake system is indicated.

What is more surprising, the ΔAf-sidA strain showed 99% decreased asexual sporulation and the capacity of A. fumigatus to establish systemic infection in a murine model was completely abolished. Likewise Af-sidA, als Af-at1, Af-at2 and Af-sidD are essential for virulence of A. fumigatus as is shown in the appended Examples. These data show that the siderophore system plays a crucial role in the pathogenicity of a fungus and makes thus the siderophore biosynthesis genes attractive targets for screening for inhibitors of the same.

As detailed herein below and as is evident for the person skilled in the art, in particular from this specification as well as from the appended examples, desired inhibitors of fungal siderophore biosynthesis may also be screened for, identified, validated and/or selected by methods carried out in vitro. These methods also comprise a method for screening inhibitors of fungal siderophore biosynthesis comprising the steps of
(a) contacting an enzyme involved in siderophore biosynthesis with a compound to be tested;
(b) determining whether said enzyme is functional in the pathway of siderophore biosynthesis in the presence of said compounds to be tested when compared to an enzyme not contacted with said compound; and
(c) identifying the compound which inhibits the enzymatic function involved in siderophore biosynthesis.

In such an assay/method it is particularly preferred that said enzyme involved in siderophore biosynthesis is present, inter alia, in form of whole cell extracts (for example extracts of A. fumigatus or cell extracts derived from cells wherein one or more enzymes identified herein and being involved/comprised in siderophore biosynthesis are heterologously expressed), in form of partially purified, in unpurified form or in purified form. It is also envisaged that said enzyme(s) is/are recombinantly expressed.

Also provided is a corresponding screening method which is useful in the detection, identification, validation, verification and/or selection of inhibitors of the siderophore biosynthesis which comprises tests/assays related to polynucleotides expressing an enzyme involved in said siderophore biosynthesis. Said method comprises in particular the steps of
(a) contacting a polynucleotide coding for an enzyme involved in siderophore biosynthesis with a compound to be tested;
(b) determining whether said polynucleotide is expressed in the presence of said compounds to be tested when compared to a second polynucleotide comprising the same nucleotide sequence which is not contacted with said compound; and
(c) identifying the compound which inhibits the functionally expression of the polynucleotide expressing an enzyme involved in siderophore biosynthesis.

Accordingly, also a method for screening inhibitors of fungal siderophore biosynthesis, based on the polynucleotides coding for an enzyme involved in the siderophore biosynthesis pathway is provided. The corresponding screening method, however, also relates to screening of inhibitors capable of interfering with the expression of the herein identified enzymes, e.g. promoter/gene expression regions, like 5′ non-translated sequences.

Before the present invention is described in detail, it is to be understood that this invention is not limited to the particular methodology, protocols, cells, vectors, reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland). Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

The methods for screening inhibitors of fungal siderophore biosynthesis which are described herein in detail are, inter alia, envisaged to be carried out in the presence of a ferrous iron chelator such as bathophenanthroline-disulfonic acid (BPS) or the like. In particular in case of the fungal species described herein (A. fumigatus) which can be used for screening, have a reductive iron uptake system. Said chelator inhibits the reductive iron uptake system, thereby enhancing the specificity of the screening method for inhibitors of fungal siderophore biosynthesis.

In a preferred embodiment of the present invention it is envisaged that the fungal siderophore biosynthesis takes place in Aspergillus species. The genus Aspergillus includes over 185 species. Accordingly, the methods for screening inhibitors of fungal siderophore biosynthesis are preferably carried out with the Aspergillus species described herein and more preferably with Aspergillus fumigatus. Around 20 species have so far been reported as causative agents of opportunistic infections in man. The Aspergillus species in which fungal siderophore biosynthesis takes place and which can be used in the methods described herein is preferably selected from the group consisting of Aspergillus flavus, Aspergillus niger, Aspergillus clavatus, Aspergillus glaucus, Aspergillus nidulans, Aspergillus ochraceus, Aspergillus oryzae, Aspergillus parasiticus, Aspergillus penicillioides, Aspergillus restrictus, Aspergillus sydowii, Aspergillus tamarii, Aspergillus terreus, Aspergillus ustus and Aspergillus versicolor.

Particularly preferred, the fungal siderophore biosynthesis takes place in Aspergillus fumigatus. Yet, Aspergillus fumigatus is preferably used in the methods described herein.

In accordance with this invention, the following test systems, A and B, may be employed for screening of inhibitors of siderophore biosynthesis at level of living cells. These test systems are by no means limiting and merely illustrative.

### A) Growth Assay:

Aspergillus fumigatus employs two high-affinity iron uptake systems, reductive iron uptake and siderophore-mediated iron uptake, which are redundantly essential for uptake of iron and therefore for growth. The reductive iron uptake system can be inhibited by the ferrous iron specific chelator bathophenanthroline disulfonic acid (BPS), making the siderophore system essential for growth. In the presence of BPS, inhibition of the siderophore biosynthesis can be monitored by reduction of growth of A. fumigatus which can be used for screening of inhibitors of siderophore biosynthesis as follows.

Microtiter plate wells containing liquid or solid Aspergillus minimal medium (Pontecorvo, Adv. Genet. 5 (1953), 141-238; Oberegger, Mol. Microbiol. 41 (2001), 1077-1089) plus 200 μM BPS with and without different inhibitors are inoculated with 102-104 conidia of A. fumigatus, incubated for 24-72 h at 37° C. and growth is scored. Inhibition of siderophore production causes inhibition of growth. Growth inhibition can be determined, e.g., by a spectrophotometrical (measuring the optical density at 620 nm with a microliter plate reader), quantitative, automated assay (Broekaert, FEMS Microbiol. Lett. 69 (1990), 55-60; Ludwig, FEMS Microbiol. Letters 69: (1990), 61-66). Specific inhibition of siderophore biosynthesis is indicated if the inhibitor causes less inhibition of growth on media if the inhibition can be antagonized by supplementation with siderophores, e.g. 10 μM ferricrocin or 10 μM triacetylfusarinine C.

Instead of BPS, also 5% sheep blood can be used, which also inhibits utilization of the reductive iron assimilation. Furthermore, instead of Aspergillus fumigatus, Aspergillus nidulans can be used for the screening—in this case no BPS has to be used because Aspergillus nidulans does not possess a reductive iron assimilatory system.

### B) Siderophore Detection

During iron starvation Aspergillus fumigatus excretes large amounts of siderophores (triacetylfusarinine C) into the growth medium, which can easily be monitored by different methods. Inhibition of siderophore biosynthesis blocks the excretion of siderophores and therefore detection of siderophores can be used for screening of inhibitors of siderophore biosynthesis as follows.

Microtiter plate wells containing liquid iron-depleted Aspergillus minimal medium (Pontecorvo, Adv. Genet. 5 (1953), 141-238; Oberegger, Mol. Microbiol. 41 (2001), 1077-1089) with and without different inhibitors are inoculated with 10²-10⁴ conidia of A. fumigatus, incubated for 24-72 h at 37° C. Siderophores in the supernatant turn red after addition of iron (end volume 100. Therefore, the supernatant of cells without inhibitors of siderophore biosynthesis turns red, whereas inhibition of siderophore biosynthesis causes a reduction of red color. Alternatively, siderophores can be monitored by e.g. the chrome azurol S (CAS) assay (Payne, Metods Enzymol. 235 (1994) 329-344). For detection of siderophores by the CAS-assay an equal volume of blue CAS-solution is added. In the presence of siderophores the blue CAS solution turns red. Therefore, the presence of siderophores is indicated by red color, whereas the presence of an inhibitor is indicated by blue colour. Furthermore, siderophores can be quantified by reversed-phase HPLC or mass spectroscopy, which also allows to determine the type of siderophore produced.

As pointed out herein, also cell-free systems may be employed in the herein described assay system. Accordingly, inhibitors of siderophores can be screened by activity assays using the polypeptides involved in siderophore biosynthesis—e.g. the polypeptides encoded by sidA, at1, sidD, rac1, at2 or at3—or fragments thereof. These polypeptides or fragments thereof catalyze reactions essential for formation of a siderophore, e.g. TAFC and ferricrocin in A. fumigatus. Inhibition of each of these enzymes causes a block of siderophore biosynthesis and therefore each of these enzymes can be used for screening of inhibitors of siderophore biosynthesis. Two illustrative examples, A and B, for screening assays are given below:

A) Screening of Inhibitors of Sida (OMO, Gene Product of sidA)

OMO (SidA) is purified from cellular extracts of A. fumigatus grown during iron starvation or purified from E. coli expressing the A. fumigatus OMO-encoding gene sidA. L-Ornithine-N⁵-oxygenase enzyme activity in the presence and absence of inhibitors is determined (Mei, Proc. Natl. Acad. Sci. 90 (1993), 903-907; Zhou, Mol. Gen. Genet. 259 (1998), 532-540). Briefly, OMO is incubated at 30° C. for 2 h in 0.1 mM potassium phosphate pH 8.0, 0.5 mM NADPH, 5 μM FAD, and 1.5 mM L-ornithine. The reaction is stopped by addition of perchloric acid to a final concentration of 66 mM. Samples are centrifuged and the supernatants are subject to the iodine oxidation test (Tomlinson, Anal. Biochem. 44 (1971), 670-679). Subsequently, the samples are briefly zentrifuged to remove denatured protein precipitates, and the absorbance at 520 nm is determined. A decrease of the absorbance is indicative for the presence of an inhibitor.

### B) Screening of Inhibitors of At2

AT2 is purified from cellular extracts of A. fumigatus grown during iron starvation or purified from E. coli expressing the A. fumigatus AT2-encoding gene. AT2 activity in the presence and absence of inhibitors is determined. Briefly, AT2 is incubated at 30° C. for 0.5 h in 0.1 mM potassium phosphate pH 8.0, 0.1 μCi of [1⁻¹⁴C]acetyl-CoA (55 mCi/mmol) and 0.1 mM fusarinine C in a final volume of 200 μl. Subsequently, synthesized triacetylfusarinine C is separated from fusarinine C by extraction into chloroform and quantified by scintillation counting. A decrease of the radioactivity in the chloroform phase is indicative for the presence of an inhibitor.

Similar experimental set-ups may be employed for the screening of inhibitors based on sidD, at1, rac1 or at3.

In accordance with the present invention, the term “inhibitor” denotes molecules or substances or compounds or compositions or agents or any combination thereof described herein below, which are capable of inhibiting and/or reducing fungal siderophore biosynthesis, particularly in Aspergillus species described herein and more particularly in Aspergillus fumigatus. The term “inhibitor” when used in the present application is interchangeable with the term “antagonist”. The term “inhibitor” comprises competitive, non-competitive, functional and chemical antagonists as described, inter alia, in Mutschler, “Arzneimittelwirkungen” (1986), Wissenschaftliche Verlagsgesellschaft mbH, Stuttgart, Germany. The term “partial inhibitor” in accordance with the present invention means a molecule or substance or compound or composition or agent or any combination thereof that is capable of incompletely blocking the action of agonists through, inter alia, a non-competitive mechanism. It is preferred that said inhibitor alters, interacts, modulates and/or prevents fungal siderophore biosynthesis in a way which leads to partial, preferably complete, standstill. Said standstill may either be reversible or irreversible.

Preferably, the inhibitor of fungal siderophore biosynthesis alters, interacts, modulates and/or prevents elements such as an enzyme involved in siderophore biosynthesis, wherein said enzyme is selected from the group consisting of L-ornithine N⁵-oxygenase, N⁵-transacylase, non-ribosomal peptide synthetase, enoyl CoA hydratase and N²-transacetylase and/or fragments thereof as described hereinbelow in detail. As is known in the art, the term “acylase” encompasses also enzymes having “acetylase” activity. In the context of the application both terms are used interchangeable.

Accordingly, the fungal species, in particular Aspergillus species described herein is, in the presence of the inhibitor, no longer capable of siderophore biosynthesis. During virulence, these A. species are no longer able to take up iron from the surrounding environment which during the course of time coincides with non-growth and, later, leads to death of the fungal species. The person skilled in the art is readily in a position to determine whether the fungal species, in particular the Aspergillus species described herein and more particularly Aspergillus fumigatus is capable of siderophore biosynthesis in the presence of an inhibitor as described herein or to be identified by the methods described herein. In particular, the appended Examples describe various assays how siderophore biosynthesis and/or inhibition of siderophore biosynthesis in Aspergillus species, in particular in Aspergillus fumigatus can be assessed. For example, Aspergillus fumigatus is no longer able to grow in the presence of an inhibitor of siderophore biosynthesis when it is grown in liquid or solid minimal medium containing 5% sheep blood (Pontecorvo (1953), Adv. Genet. 5, 141-238). Specific inhibition of siderophore synthesis is indicated if the inhibitor causes less inhibition of growth on media without blood or if the inhibition can be antagonized by supplementation with siderophores as is described in Examples 15, 28 and 29. A possibility to enhance specificity of siderophore biosynthesis in Aspergillus fumigatus is provided by employing ferrous iron chelators, such as bathophenanthroline disulfonic acid (BPS) which inhibits the reductive iron uptake system of Aspergillus species, in particular that of Aspergillus fumigatus. Alternatively, inhibition of siderophore biosynthesis can also be determined by the CAS-assay, HPLC-analysis or mass spectroscopy as is described in Example 15. The present invention also provides screening methods for inhibitors of siderophore biosynthesis in Aspergillus nidulans as well as assays to determine whether Aspergillus nidulans is capable of siderophore biosynthesis in the presence of an inhibitor of siderophore biosynthesis which are described in Examples 15, 17, 28 and 29. It is to be understood that the aforementioned assays for determining whether the fungal species used in the screening methods for inhibitors of siderophore biosynthesis is capable of siderophore biosynthesis or not are also useful for determining whether any of the elements, e.g., any of the enzymes described herein involved in siderophore biosynthesis is, e.g., inhibited by a potential inhibitor as described herein.

The term “siderophore biosynthesis” which is interchangeable with the term “biosynthesis of a siderophore” or “fungal siderophore biosynthesis” when used in the present invention means all elements such as preferably the enzymes described hereinbelow of the biosynthetic pathway which is involved in the synthesis of siderophores. Said term also comprises elements, such as transporters or channels or the like which secrete either actively or passively siderophores produced from intracellular to extracellular milieu and it comprises elements which are involved in the uptake and transport of secreted siderophores from extracellular milieu to intracellular milieu. Moreover, said term comprises elements involved in uncoupling or detaching iron from a siderophore as well as elements involved in channeling in iron into the metabolism of a fungal cell, wherein said iron is taken up in the extracellular milieu by a siderophore and is transported to the intracellular milieu as described above. The proposed biosynthetic pathway of siderophore biosynthesis is described in Haas (2003), loc. cit. and, for example, shown in the appended FIG. 8. Yet, it is of note that besides the elements shown in FIG. 8 further elements of the siderophore biosynthesis pathway are involved.

Siderophores are low molecular iron specific chelators as described herein.

“A cell expressing a fungal siderophore” is a cell as described hereinbelow which is capable of biosynthesis of a fungal siderophore. Said cell may be a fungal cell but said cell may also comprise a cell which heterologously expresses an enzyme involved in the siderophore biosynthesis as provided herein. Cells to be employed may be selected from the group consisting of an animal cell, e.g., a mammalian cell, insect cell, amphibian cell or fish cell, a plant cell, fungal cell and bacterial cell as will be described in more detail hereinbelow. As documented herein, also whole cell extracts may be employed in the screening methods provided herein. Also envisaged is the use of the unpurified, partially purified, purified or recombinantly expressed enzymes comprised in the siderophore biosynthesis pathway and disclosed herein.

The person skilled in the art can easily employ the compounds and the methods of this invention in order to elucidate the inhibitory effects and/or characteristics of a test compound to be identified and/or characterized in accordance with any of the methods described herein and which is an inhibitor of fungal siderophore biosynthesis.

The term “test compound” or “compound to be tested” refers to a molecule or substance or compound or composition or agent or any combination thereof to be tested by one or more screening method(s) of the invention as a putative inhibitor of fungal siderophore biosynthesis. A test compound can be any chemical, such as an inorganic chemical, an organic chemical, a protein, a peptide, a carbohydrate, a lipid, or a combination thereof or any of the compounds, compositions or agents described herein. It is to be understood that the term “test compound” when used in the context of the present invention is interchangeable with the terms “test molecule”, “test substance”, “potential candidate”, “candidate” or the terms mentioned hereinabove.

Accordingly, small peptides or peptide-like molecules as described hereinbelow are envisaged to be used in the screening methods for inhibitor(s) of fungal siderophore biosynthesis. Such small peptides or peptide-like molecules bind to and occupy the active site of a protein thereby making the catalytic site inaccessible to substrate such that normal biological activity is prevented. Moreover, any biological or chemical composition(s) or substance(s) may be envisaged as fungal siderophore biosynthesis inhibitor. The inhibitory function of the inhibitor can be measured by methods known in the art and by methods described herein. Such methods comprise interaction assays, like immunoprecipitation assays, ELISAs, RIAs as well as specific inhibition assays, like the assays provided in the appended examples (e.g. enzymatic in vitro assays) and inhibition assays for gene expression. In the context of the present application it is envisaged that cells expressing a fungal siderophore as described herein are used in the screening assays. It is also envisaged that elements of the pathway of siderophore biosynthesis may be used, e.g., enzymes. Said enzymes may be present in whole cell extracts of cells expressing a fungal siderophore or said enzymes may be purified, partially purified or recombinantly expressed as described hereinbelow. Accordingly and as documented in the examples (e.g. example 16 or 29) the herein provided screening assays also relate to enzymatic in vitro tests. Also preferred potential candidate molecules or candidate mixtures of molecules to be used when contacting a cell expressing a fungal siderophore or an element of the fungal siderophore biosynthesis pathway, particularly of an Aspergillus species as described herein, more preferably of Aspergillus fumigatus, may be, inter alia, substances, compounds or compositions which are of chemical or biological origin, which are naturally occurring and/or which are synthetically, recombinantly and/or chemically produced. Thus, candidate molecules may be proteins, protein-fragments, peptides, amino acids and/or derivatives thereof or other compounds, such as ions, which bind to and/or interact with elements, such as metabolites, intermediates or enzymes of the biosynthesis pathway for fungal siderophores, in particular with enzymes selected from the group consisting of L-ornithine N⁵-oxygenase, N⁵-transacylase, non-ribosomal peptide synthetase, and N²-transacetylase and/or fragments thereof which are described hereinbelow in detail. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means.

In addition, the generation of chemical libraries is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds can theoretically be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallon, Journal of Medicinal Chemistry, Vol. 37, No. 9, 1233-1250 (1994)). Other chemical libraries known to those in the art may also be used, including natural product libraries. Once generated, combinatorial libraries are screened for compounds that possess desirable biological properties. For example, compounds which may be useful as drugs or to develop drugs would likely have the ability to bind to the target protein identified, expressed and purified as described herein.

In the context of the present invention, libraries of compounds are screened to identify compounds that function as inhibitors of the target gene product, here elements of the pathway for fungal siderophore biosynthesis. First, a library of small molecules is generated using methods of combinatorial library formation well known in the art. U.S. Pat. Nos. 5,463,564 and 5,574,656 are two such teachings. Then the library compounds are screened to identify those compounds that possess desired structural and functional properties. U.S. Pat. No. 5,684,711, discusses a method for screening libraries. To illustrate the screening process, the target cell or gene product and chemical compounds of the library are combined and permitted to interact with one another. A labeled substrate is added to the incubation. The label on the substrate is such that a detectable signal is emitted from metabolized substrate molecules. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the cell/enzyme can be analyzed and features common to the various compounds identified can be isolated and combined into future iterations of libraries. Once a library of compounds is screened, subsequent libraries are generated using those chemical building blocks that possess the features shown in the first round of screen to have activity against the target cell/enzyme. Using this method, subsequent iterations of candidate compounds will possess more and more of those structural and functional features required to inhibit the function of the target cell/enzyme, until a group of (enzyme) inhibitors with high specificity for the enzyme can be found. These compounds can then be further tested for their safety and efficacy as antibiotics for use in animals, such as mammals. It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads, in particular for the inhibition of siderophore biosynthesis. Such techniques include the methods described in PCT publications No. WO 99/35494, WO 98/19162, WO 99/54728.

Preferably, candidate agents encompass numerous chemical classes, though typically they are organic molecules, preferably small organic compounds having a molecular weight of more than 50 and less than about 2,500 Daltons, preferably less than about 750, more preferably less than about 350 daltons.

Candidate agents may also comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Exemplary classes of candidate agents may include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

As mentioned above, candidate agents are also found among biomolecules including peptides, amino acids, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Other candidate compounds to be used as a starting point for the screening of inhibitors of fungal siderophore biosynthesis are aptamers, aptazymes, RNAi, shRNA, RNAzymes, ribozymes, antisense DNA, antisense oligonucleotides, antisense RNA, antibodies, affibodies, trinectins, anticalins, or the like compounds which are described in detail hereinbelow. Target sequences on the nucleotide level are illustratively given herein below and comprise, but are not limited to target nucleotide sequences comprising or being the sequences shown in SEQ ID NOS: 72, 75, 78, 81, 84, 87, 90, 93, 96, 99, 102, 105, 108, 111, 114, 117, 120, 123, 126, 129, 132, 135, 138, 141, 144, 147, 150, 153, 156 and 159.

Accordingly, the person skilled in the art is readily in a position to have candidate compounds at his disposal which can be used in the screening methods for inhibitors of fungal siderophore biosynthesis as a basis to, inter alia, improve or further develop the capability of such compounds to inhibit fungal siderophore biosynthesis. Accordingly, the person skilled in the art can readily modify such compounds by methods known in the art to improve their capability of acting as an inhibitor in the sense of the present invention. The capability of one or more of the aforementioned compounds to inhibit fungal siderophore biosynthesis, preferably in an Aspergillus spec., more preferably in Aspergillus fumigatus is tested as described hereinabove.

In one embodiment of the present invention, the enzymes involved in fungal siderophore biosynthesis as described herein are isolated and expressed. These recombinant proteins are then used as targets in assays to screen libraries of compounds for potential drug candidates. Corresponding embodiments are described herein and are also given in the appended examples.

It will be appreciated by those skilled in the art that Aspergillus fumigatus strains which synthesize a siderophore as described herein are used to develop in vitro assays for screening for inhibitors of siderophore biosynthesis. Of, course, also whole cell extracts of such Aspergillus fumigatus strains are envisaged to be used for the screening assays described herein. Yet, as mentioned above, also cell-based screening assays are within the scope of the present invention.

Current cell-based assays used to identify or to characterize compounds for drug discovery and development frequently depend on detecting the ability of a test compound to modulate the activity of a target molecule located within a cell or located on the surface of a cell. Most often such target molecules are proteins such as enzymes, receptors and the like. A number of highly sensitive cell-based assay methods are available to those of skill in the art to detect binding and interaction of test compounds with specified-target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment such as the periplasm of a bacterial cell. Thus, current cell-based assay methods are limited in that they are not effective in identifying or characterizing compounds that interact with their targets with moderate to low affinity or compounds that interact with targets that are not readily accessible. The cell-based assay methods of the present invention have substantial advantages over current cell-based assays. These advantages derive from the use of sensitized cells in which the level or activity of at least one gene product required for fungal siderophore biosynthesis and, thus, for virulence and/or pathogenicity has been specifically reduced to the point where the presence or absence of its function becomes a rate-determining step for fungal siderophore biosynthesis. Such sensitized cells become much more sensitive to compounds that are active against the affected target molecule. For example, sensitized cells are obtained by growing a conditional-expression Aspergillus fumigatus mutant strain in the presence of a concentration of inducer or repressor which provides a level of a gene product required for fungal siderophore biosynthesis such that the presence or absence of its function becomes a rate-determining step for fungal siderophore biosynthesis. Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells.

Current methods employed in the arts of medicinal and combinatorial chemistry are able to make use of structure-activity relationship information derived from testing compounds in various biological assays including direct binding assays and cellbased assays. Occasionally compounds are directly identified in such assays that are sufficiently potent to be developed as drugs. More often, initial hit compounds exhibit moderate or low potency. Once a hit compound is identified with low. or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus, more compounds can now be characterized providing more useful information than would be obtained otherwise.

In a preferred embodiment of the present invention the siderophore biosynthesis involves one or more enzymes selected from the group consisting of L-ornithine N⁵-oxygenase, N⁵-transacylase, non-ribosomal peptide synthetase, enoyl CoA hydratase and N²-transacetylase and/or fragments thereof. It is envisaged that inhibiting, altering and/or modulating said enzymes leads to stillstand of fungal siderophore biosynthesis when said one or more enzymes are contacted with an inhibitor or a candidate compound as described herein which is identified as being an inhibitor of fungal siderophore biosynthesis according to the methods for screening of the present invention. The present invention also provides for nucleic acid molecules encoding the above recited enzymes involved in the (fungal) siderophore biosynthesis. As pointed out above, these enzymes (and fragments thereof) as well as the corresponding polynucleotides (comprising also 5′ untranslated regions and/or gene regulatory sequences) are particularly useful in the screening methods provided herein. Said screening methods are particularly useful in the detection, identification, validation as well as verification of inhibitors of the siderophore biosynthesis.

In a more preferred embodiment said L-ornithine N⁵-oxygenase is encoded by a polynucleotide (which is also referred to herein as Af-sidA or sidA) comprising the sidA gene of Aspergillus fumigatus. More preferably said L-ornithine N⁵-oxygenase is encoded by a polynucleotide comprising the nucleic acid sequence selected from the group consisting of:
(a) the nucleic acid sequence set forth in SEQ ID NO: 1;
(b) a nucleic acid sequence encoding a polypeptide comprising the amino acid sequence set forth in SEQ ID NO: 2 or encoding a polypeptide which is at least 70, 80, 90, 95, 96, 97, 98 or 99%, preferably at least 86% identical to the amino acid sequence set forth in SEQ ID NO: 2 and which has L-ornithine N⁵-monooxygenase activity;
(c) a nucleic acid sequence encoding a fragment of the polypeptide comprising the amino acid sequence set forth in SEQ ID NO: 2 said fragment showing L-ornithine N⁵-monooxygenase activity;
(d) a nucleic acid sequence which is at least 70, 80, 90, 95, 96, 97, 98 or 99% and preferably at least 85%, identical to a polynucleotide as defined in any one of (a) to (c) and which encodes a polypeptide with L-ornithine N⁵-monooxygenase activity; and (e) a nucleic acid sequence the complementary strand of which hybridizes to a nucleic acid sequence as defined in any one of (a) to (c) and which encodes a polypeptide with L-ornithine N⁵-monooxygenase activity;
   or the complementary strand of such a nucleic acid sequence.

L-ornithine N⁵-monooxygenase is the committed step enzyme in siderophore biosynthesis. Accordingly, its activity can be determined by evaluating whether an organism or cell, preferably an Aspergillus species or an Aspergillus species cell, more preferably Aspergillus fumigatus or an Aspergillus fumigatus cell, normally expressing a fungal siderophore, which lacks L-ornithine N⁵-monooxygenase or has a non-functional L-ornithine N⁵-monooxygenase and which comprises and/or expresses the aforementioned nucleic acid molecule is capable to express again a siderophore as is described herein. Such an activity test is known in the art, e.g., as “functional complementation”:

L-ornithine N⁵-monooxygenase activity can also be determined by observation of the conversion of L-ornithine to N⁵-hydroxy-L-ornithine which is assayed as described in Example 16.

In another preferred embodiment the N⁵-transacylase is encoded by a polynucleotide comprising the nucleic acid sequence selected from the group consisting of:
(a) the nucleic acid sequence set forth in SEQ ID NO: 3;
(b) a nucleic acid sequence encoding a polypeptide comprising the amino acid sequence set forth in SEQ ID NO: 4 or encoding a polypeptide which is at least 70, 80, 90, 95, 96, 97, 98 or 99%, preferably at least 70% identical to the amino acid sequence set forth in SEQ ID NO: 4 and which has N⁵-transacylase activity;
(c) a nucleic acid sequence encoding a fragment of the polypeptide comprising the amino acid sequence set forth in SEQ ID NO: 4 said fragment showing N⁵-transacylase activity;
(d) a nucleic acid sequence which is at least 70, 80, 90, 95, 96, 97, 98 or 99%, identical to a polynucleotide as defined in any one of (a) to (c) and which encodes a polypeptide with N⁵-transacylase activity; and
(e) a nucleic acid sequence the complementary strand of which hybridizes to a nucleic acid sequence as defined in any one of (a) to (c) and which encodes a polypeptide with N⁵-transacylase activity;
   or the complementary strand of such a nucleic acid sequence.

N⁵-transacylase activity can be determined by evaluating whether an organism or cell, preferably an Aspergillus species or an Aspergillus species cell, more preferably Aspergillus fumigatus or an Aspergillus fumigatus cell, normally expressing a fungal siderophore, which lacks N⁵-transacylase or has a non-functional N⁵-transacylase and which comprises and/or expresses the aforementioned nucleic acid molecule is capable to express again a siderophore as is described herein.

N⁵-transacylase activity can also be determined by as described in Example 15.

In another preferred embodiment the non-ribosomal peptide synthetase is encoded by a polynucleotide (which is also referred to herein as Af-sidD or sidD) comprising the nucleic acid sequence selected from the group consisting of:
(a) the nucleic acid sequence set forth in SEQ ID NO: 5;
(b) a nucleic acid sequence encoding a polypeptide comprising the amino acid sequence set forth in SEQ ID NO: 6 or encoding a polypeptide which is at least 70, 80, 90, 95, 96, 97, 98 or 99%, preferably at least 80% identical to the amino acid sequence set forth in SEQ ID NO: 6 and which has non-ribosomal peptide synthetase activity;
(c) a nucleic acid sequence encoding a fragment of the polypeptide comprising the amino acid sequence set forth in SEQ ID NO: 6 said fragment showing non-ribosomal peptide synthetase activity;
(d) a nucleic acid sequence which is at least 70, 80, 90, 95, 96, 97, 98 or 99% and preferably at least 89% identical to a polynucleotide as defined in any one of (a) to (c) and which encodes a polypeptide with non-ribosomal peptide synthetase activity; and
(e) a nucleic acid sequence the complementary strand of which hybridizes to a nucleic acid sequence as defined in any one of (a) to (c) and which encodes a polypeptide with non-ribosomal peptide synthetase activity;
   or the complementary strand of such a nucleic acid sequence.

Non-ribosomal peptide synthetase activity can be determined by evaluating whether an organism or cell, preferably an Aspergillus species or an Aspergillus species cell, more preferably Aspergillus fumigatus or an Aspergillus fumigatus cell, normally expressing a fungal siderophore, which lacks non-ribosomal peptide synthetase or has a non-functional non-ribosomal peptide synthetase and which comprises and/or expresses the aforementioned nucleic acid molecule is capable to express again a siderophore as is described herein.

Non-ribosomal peptide synthetase activity can also be determined as is described in Example 15. As described in the appended Examples, in particular Examples 21 and 22 herein, to elucidate the function of sidD, a deletion mutant for this gene was constructed. In the generated mutant allele of ΔsidD the region encompassing amino acids 305-1120 was replaced by the hygromycin resistance (hph) marker. Reversed-phase-HPLC according to Oberegger (Mol. Microbiol. 41 (2001), 1077-1089) demonstrated that the ΔsidD strain lost the ability to produce the extracellular siderophore triacetylfusarinine C but still produced the intracellular siderophore ferricrocin (FIG. 11). These data further show that different nonribosomal peptide synthetases are required for synthesis of triacetylfusarinine C and ferricrocin. Compared to the wild type (wt), ΔsidD displayed no significant differences in radial growth during iron-replete and iron depleted conditions but a significant reduced growth rate on blood agar and during iron depleted conditions in the presence of bathophenantroline-disulfonic acid (FIG. 12), which is consistent with reductive iron assimilation being responsible for normal growth during iron-replete and iron-depleted conditions. The capacity to establish systemic infection in a murine model of the ΔsidD was significantly reduced (FIG. 13), demonstrating that the extracellular siderophore triacetylfusarinine C plays a crucial role in virulence of A. fumigatus and makes thus the enzymes of the underlying biosynthetic pathway an attractive target for development of screening for inhibitors of the same (see, in particular, Example 27).

In a further preferred embodiment the enoyl CoA hydratase is encoded by a polynucleotide (herein also referred to as Af-rac1 or rac1) comprising a nucleic acid sequence selected from the group consisting of:
(a) the nucleic acid sequence set forth in SEQ ID NO: 7;
(b) a nucleic acid sequence encoding a polypeptide comprising the amino acid sequence set forth in SEQ ID NO: 8 or encoding a polypeptide which is at least 70, 80, 90, 95, 96, 97, 98 or 99%, preferably at least 91% identical to the amino acid sequence set forth in SEQ ID NO: 8 and which has enoyl CoA hydratase activity;
(c) a nucleic acid sequence encoding a fragment of the polypeptide comprising the amino acid sequence set forth in SEQ ID NO: 8 said fragment showing enoyl CoA hydratase activity;
(d) a nucleic acid sequence which is at least 70, 80, 90, 95, 96, 97, 98 or 99% identical to a polynucleotide as defined in any one of (a) to (c) and which encodes a polypeptide with enoyl CoA hydratase activity; and
(e) a nucleic acid sequence the complementary strand of which hybridizes to a nucleic acid sequence as defined in any one of (a) to (c) and which encodes a polypeptide with enoyl CoA hydratase activity;
   or the complementary strand of such a nucleic acid sequence.

Enoyl CoA hydratase activity can be determined by evaluating whether an organism or cell, preferably an Aspergillus species or an Aspergillus species cell, more preferably Aspergillus fumigatus or an Aspergillus fumigatus cell, normally expressing a fungal siderophore, which lacks enoyl CoA hydratase or has a non-functional enoyl CoA hydratase and which comprises and/or expresses the aforementioned nucleic acid molecule is capable to express again a siderophore as is described herein.

Enoyl CoA activity can also be determined as described in Example 15.

Northern analysis demonstrated that similar to sidA, at1, sidD, and at2, the expression of rac1 (encoding the putative enoyl-CoA-hydratase, was induced during iron-depleted conditions, which suggested involvement of the gene products in iron metabolism of A. fumigatus.

As described in the appended Examples herein, to elucidate the function of rac1, a deletion mutant for this gene was constructed using a similar strategy as for generation of the ΔsidA deletion mutant. In the generated mutant allele of rac1 the region encoding amino acids 17-261 was replaced by the hygromycin resistance (hph) marker. Reversed-phase-HPLC according to Oberegger (Mol. Microbiol. 41 (2001), 1077-1089) demonstrated that the Δrac1 strain lost the ability to produce the extracellular siderophore triacetylfusarinine C but still produced the intracellular siderophore ferricrocin. Because the capacity to establish systemic infection in a murine model of all A. fumigatus mutants lacking the extracellular siderophore TAFC (sidA, at1, sidD, and at2) was significantly reduced, it can be expected that Δrac1 also has reduced virulence. Therefore, Af-rac1 is an attractive target for development of screening for inhibitors of the same.

In a further preferred embodiment the N²-transacetylase which may also have N⁵-transacylase activity is encoded by a polynucleotide (which is also referred to herein as Af-at1 or at1) comprising a nucleic acid sequence selected from the group consisting of:
(a) the nucleic acid sequence set forth in SEQ ID NO: 9;
(b) a nucleic acid sequence encoding a polypeptide comprising the amino acid sequence set forth in SEQ ID NO: 10 or encoding a polypeptide which is at least 70, 80, 90, 95, 96, 97, 98 or 99%, preferably at least 90% identical to the amino acid sequence set forth in SEQ ID NO: 10 and which has N²-transacetylase activity;
(c) a nucleic acid sequence encoding a fragment of the polypeptide comprising the amino acid sequence set forth in SEQ ID NO: 10 said fragment showing N²-transacetylase activity;
(d) a nucleic acid sequence which is at least 70, 80, 90, 95, 96, 97, 98 or 99% and preferably at least 84% identical to a polynucleotide as defined in any one of (a) to (c) and which encodes a polypeptide with N²-transacetylase activity; and
(e) a nucleic acid sequence the complementary strand of which hybridizes to a nucleic acid sequence as defined in any one of (a) to (c) and which encodes a polypeptide with N²-transacetylase activity;
   or the complementary strand of such a nucleic acid sequence.

N²-transacetylase activity can be determined by evaluating whether an organism or cell, preferably an Aspergillus species or an Aspergillus species cell, more preferably Aspergillus fumigatus or an Aspergillus fumigatus cell, normally expressing a fungal siderophore, which lacks N²-transacetylase or has a non-functional N²-transacetylase and which comprises and/or expresses the aforementioned nucleic acid molecule is capable to express again a siderophore as is described herein.

Activity of Af-at1 can also be determined as described in Example 15.

As described in the appended Examples herein, to elucidate the function of at1, a deletion mutant for this gene was constructed using a similar strategy as for generation of the ΔsidA deletion mutant. In the generated mutant allele of at1 the region encoding amino acids 5-451 was replaced by the hygromycin resistance (hph) marker. Reversed-phase-HPLC according to Oberegger (Mol. Microbiol. 41 (2001), 1077-1089) demonstrated that the transacylase deficient Δat1 strain lost the ability to produce the extracellular siderophore triacetylfusarinine C but still produced the intracellular siderophore ferricrocin (FIG. 11). Compared to the wild type (wt), ΔAf-at1 displayed no significant differences in radial growth during iron-replete and iron depleted conditions, but a significant reduced growth rate on blood agar and during iron depleted conditions in the presence of bathophenantroline-disulfonic acid (FIG. 12), which is consistent with reductive iron assimilation being responsible for the normal growth during iron-replete and iron-depleted conditions. The capacity to establish systemic infection in a murine model of the Δat1 was significantly reduced (FIG. 13), demonstrating that the extracellular siderophore triacetylfusarinine C plays a crucial role in virulence of A. fumigatus and makes thus the enzymes of the underlying biosynthetic pathway an attractive target for development of screening for inhibitors of the same.

In another preferred embodiment an N²-transacetylase is encoded by a polynucleotide (which is also referred to herein as Af-at2 or at2) comprising a nucleic acid sequence selected from the group consisting of:
(a) the nucleic acid sequence set forth in SEQ ID NO: 16;
(b) a nucleic acid sequence encoding a polypeptide comprising the amino acid sequence set forth in SEQ ID NO: 17 or encoding a polypeptide which is at least 70, 80, 90, 95, 96, 97, 98 or 99%, preferably at least 90% identical to the amino acid sequence set forth in SEQ ID NO: 17 and which has N²-transacetylase activity;
(c) a nucleic acid sequence encoding a fragment of the polypeptide comprising the amino acid sequence set forth in SEQ ID NO: 17 said fragment showing N²-transacetylase activity;
(d) a nucleic acid sequence which is at least 70, 80, 90, 95, 96, 97, 98 or 99% and preferably at least 84% identical to a polynucleotide as defined in any one of (a) to (c) and which encodes a polypeptide with N²-transacetylase activity; and
(e) a nucleic acid sequence the complementary strand of which hybridizes to a nucleic acid sequence as defined in any one of (a) to (c) and which encodes a polypeptide with N²-transacetylase activity;
   or the complementary strand of such a nucleic acid sequence.

N²-transacetylase activity can be determined by evaluating whether an organism or cell, preferably an Aspergillus species or an Aspergillus species cell, more preferably Aspergillus fumigatus or an Aspergillus fumigatus cell, normally expressing a fungal siderophore, which lacks N²-transacetylase or has a non-functional N²-transacetylase and which comprises and/or expresses the aforementioned nucleic acid molecule is capable to express again a siderophore as is described herein.

Activity of Af-at2 can also be determined as described in Example 15.

As described in the appended Examples herein, to elucidate the function of at2, a deletion mutant for this gene was constructed. In the generated mutant allele of Δat2 the entire coding region, 117 bp of the 3′-downstream and 113 bp of the 5′-upstream region of at2 was replaced by the hygromycin resistance (hph) marker. Reversed-phase-HPLC according to Oberegger (Mol. Microbiol. 41 (2001), 1077-1089) demonstrated that the Δat2 strain lost the ability to produce the extracellular siderophore triacetylfusarinine C but still produced the intracellular siderophore ferricrocin. However, Δat2 excreted fusarinine C in amounts comparable to the wild type triacetylfusarinine production (FIG. 11). This finding suggests that the enzyme encoded by at2 is involved in conversion of fusarinine C into triacetylfusarinine C (FIG. 8). Compared to the wild type (wt), ΔAf-at1 displayed no significant differences in radial growth during iron-replete, iron depleted conditions, on blood agar or during iron depleted conditions in the presence of bathophenantroline-disulfonic acid (FIG. 12). The comparison of radial growth of the wild type and Δat2 demonstrated that under all conditions tested fusarinine C appears to fulfill the same function as triacetylfusarinine C. However, the capacity to establish systemic infection in a murine model of the Δat2 was significantly reduced (FIG. 13), demonstrating that the extracellular siderophore triacetylfusarinine C plays a crucial role in virulence of A. fumigatus and cannot be replaced by its precursor fusarinine C. This finding makes thus the enzymes of the underlying biosynthetic pathway an attractive target for development of screening for inhibitors of the same

Interestingly, the genes encoding the enzymes involved in siderophore biosynthesis of Aspergillus fumigatus as described herein are clustered with the exception of sidA. Moreover, said genes are upregulated by iron through the transcription factor SreA. The sequence of the gene cluster comprising the open reading frames of the genes encoding N⁵-transacylase, non-ribosomal peptide synthetase, enoyl CoA hydratase and N²-transacetylase of Aspergillus fumigatus is shown in SEQ ID NO: 11.

The gene comprising the genomic region encoding N⁵-transacylase (at3) of Aspergillus fumigatus is shown in SEQ ID NO: 12 and is located at position 3942 to 5066 of SEQ ID NO: 11.

The gene comprising the genomic region encoding non-ribosomal peptide synthetase (sidD) of Aspergillus fumigatus is shown in SEQ ID NO: 13 and is located at position 9908 to 16124 of SEQ ID NO: 11.

The gene comprising the genomic region encoding enoyl CoA hydratase (rac1) of Aspergillus fumigatus is shown in SEQ ID NO: 14 and is located at position 8314 to 9199 of SEQ ID NO: 11.

The gene comprising the genomic region encoding N²-transacetylase (at1) of Aspergillus fumigatus is shown in SEQ ID NO: 15 and is located at position 6614 to 8002 of SEQ ID NO: 11.

The gene comprising the genomic region encoding N²-transacetylase (at2) of Aspergillus fumigatus is shown in SEQ ID NO: 16.

SEQ ID NO: 11 also provides for 5′ non-translated regions of the genes identified herein. Also those regions are useful in the screening methods of the present invention, in particular, when inhibitors are to be identified, detected, validated and/or verified which prevent or impair the corresponding gene expression. For example, nucleotides 5067 to 6013 of SEQ ID NO: 11 correspond to a gene expression/promoter sequence of at3. A similar promoter region for rac1 and at1 is shown at position 8003 to 8313 of SEQ ID NO: 11. A sidD promoter region is depicted between position 9200 to 9907 of SEQ ID NO: 11.

It is to be understood that the aforementioned genomic regions may comprise one or more introns or non-coding sequences which, during the process of transcription and/or translation are, e.g., spliced out to give rise to a transcript which encodes N⁵-transacylase, non-ribosomal peptide synthetase, enoyl CoA hydratase or N²-transacetylase of Aspergillus fumigatus. It is also of note that one or more of the above described genomic regions may be orientated in sense or antisense orientation. The person skilled in the art is readily in a position to determine the sense or antisense orientation by means and methods known in the art. Moreover, it is envisaged that the aforementioned and below mentioned embodiments pertaining to nucleic acid and/or amino sequences apply to the sequences shown in SEQ ID NOs: 11 to 15, mutatis mutandis. The nucleic acid molecules described herein and coding for specific gene products involved in the fungal siderophore biosynthesis pathway are not only useful as drug targets, but may also be employed in diagnostic methods provided herein. The same applies, mutatis mutandis, to the corresponding gene products provided in this invention.

In accordance with the present invention, the term “nucleic acid sequence” means the sequence of bases comprising purine- and pyrimidine bases which are comprised by nucleic acid molecules, whereby said bases represent the primary structure of a nucleic acid molecule. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

When used herein, the term “polypeptide” means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine (Se-Cys). Peptides, oligopeptides and proteins may be termed polypeptides. The terms polypeptide and protein are often used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature.

In order to determine whether a nucleic acid sequence has a certain degree of identity to the nucleic acid sequence encoding L-ornithine N⁵-monooxygenase, N⁵-transacylase, non-ribosomal peptide synthetase, enoyl CoA hydratase or N²-transacetylase, the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be, used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

% sequence identity×% maximum BLAST score/100

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

The present invention also relates to nucleic acid molecules which hybridize to one of the above described nucleic acid molecules and which has L-ornithine N⁵-monooxygenase activity, N⁵-transacylase, non-ribosomal peptide synthetase activity, enoyl CoA hydratase or N²-transacetylase activity.

The term “hybridizes” as used in accordance with the present invention may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which code for L-ornithine N⁵-monooxygenase, non-ribosomal peptide synthetase, N²-transacetylase, N⁵-transacylase or enoyl-CoA hydratase and which have a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with a nucleic acid sequence as described above encoding L-ornithine N⁵-monooxygenase, N⁵-transacylase, non-ribosomal peptide synthetase, enoyl CoA hydratase or N²-transacetylase. Moreover, the term “hybridizing sequences” preferably refers to sequences encoding L-ornithine N⁵-monooxygenase, N⁵-transacylase, non-ribosomal peptide synthetase, enoyl CoA hydratase or N²-transacetylase having a sequence identity of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with an amino acid sequence of L-ornithine N⁵-monooxygenase, N⁵-transacylase, non-ribosomal peptide synthetase, enoyl CoA hydratase or N²-transacetylase as described herein above.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands.

Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid.

The nucleic acid molecule according to the invention may be any type of nucleic acid, e.g. DNA, RNA or PNA (peptide nucleic acid).

For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by Nielsen et al., Science 254:1497 (1991); and Egholm et al., Nature 365:666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the strong binding. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

The DNA may, for example, be cDNA. In a preferred embodiment it is a genomic DNA. The RNA may be, e.g., mRNA. The nucleic acid molecule may be natural, synthetic or semisynthetic or it may be a derivative, such as peptide nucleic acid (Nielsen, Science 254 (1991), 1497-1500) or phosphorothioates. Furthermore, the nucleic acid molecule may be a recombinantly produced chimeric nucleic acid molecule comprising any of the aforementioned nucleic acid molecules either alone or in combination.

Preferably, the nucleic acid molecule(s) of the present invention is part of a vector. Therefore, the present invention relates in another embodiment to a vector comprising the nucleic acid molecule of this invention. Such a vector may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

The nucleic acid molecules of the present invention may be inserted into several commercially available vectors. Nonlimiting examples include plasmid vectors compatible with mammalian cells, such as pUC, pBluescript (Stratagene), pET (Novagen), pREP (Invitrogen), pCRTopo (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pUCTag, pIZD35, pLXIN and PSIR (Clontech) and pIRES-EGFP (Clontech). Baculovirus vectors such as pBlueBac, BacPacz Baculovirus Expression System (CLONTECH), and MaxBac™ Baculovirus Expression System, insect cells and protocols (Invitrogen) are available commercially and may also be used to produce high yields of biologically active protein. (see also, Miller (1993), Curr. Op. Genet. Dev., 3, 9; O'Reilly, Baculovirus Expression Vectors: A Laboratory Manual, p. 127). In addition, prokaryotic vectors such as pcDNA2; and yeast vectors such as pYes2 are nonlimiting examples of other vectors suitable for use with the present invention. For vector modification techniques, see Sambrook and Russel (2001), loc. cit. Vectors can contain one or more replication and inheritance systems for cloning or expression, one or more markers for selection in the host, e.g., antibiotic resistance, and one or more expression cassettes.

The coding sequences inserted in the vector can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

Furthermore, the vectors may, in addition to the nucleic acid sequences of the invention, comprise expression control elements, allowing proper expression of the coding regions in suitable hosts. Such control elements are known to the artisan and may include a promoter, translation initiation codon, translation and insertion site or internal ribosomal entry sites (IRES) (Owens, Proc. Natl. Acad. Sci. USA 98 (2001), 1471-1476) for introducing an insert into the vector. Preferably, the nucleic acid molecule of the invention is operatively linked to said expression control sequences allowing expression in eukaryotic or prokaryotic cells.

Control elements ensuring expression in eukaryotic and prokaryotic cells are well known to those skilled in the art. As mentioned above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous sarcome virus), human elongation factor 10-promoter, CMV enhancer, CaM-kinase promoter or SV40-enhancer.

For the expression in prokaryotic cells, a multitude of promoters including, for example, the tac-lac-promoter, the lacUV5 or the trp promoter, has been described. Beside elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-Vitrogene, as used, inter alia in the appended examples), pSPORT1 (GIBCO BRL) or pGEMHE (Promega), or prokaryotic expression vectors, such as lambda gt11.

An expression vector according to this invention is at least capable of directing the replication, and preferably the expression, of the nucleic acids and protein of this invention. Suitable origins of replication include, for example, the Col E1, the SV40 viral and the M 13 origins of replication. Suitable promoters include, for example, the cytomegalovirus (CMV) promoter, the lacZ promoter, the gal10 promoter and the Autographa californica multiple nuclear polyhedrosis virus (AcMNPV) polyhedral promoter. Suitable termination sequences include, for example, the bovine growth hormone, SV40, lacZ and AcMNPV polyhedral polyadenylation signals. Examples of selectable markers include neomycin, ampicillin, and hygromycin resistance and the like. Specifically-designed vectors allow the shuttling of DNA between different host cells, such as bacteria-yeast, or bacteria-animal cells, or bacteria-fungal cells, or bacteria or invertebrate cells.

Beside the nucleic acid molecules of the present invention, the vector may further comprise nucleic acid sequences encoding secretion signals. Such sequences are well known to the person skilled in the art. Furthermore, depending on the expression system used leader sequences capable of directing the expressed polypeptide to a cellular compartment may be added to the coding sequence of the nucleic acid molecules of the invention and are well known in the art. The leader sequence(s) is (are) assembled in appropriate phase with translation, initiation and termination sequences, and preferably, a leader sequence capable of directing secretion of translated protein, or a part thereof, into, inter alia, the extracellular membrane. Optionally, the heterologous sequence can encode a fusion protein including an C- or N-terminal identification peptide imparting desired characteristics, e.g., stabilization or simplified purification of expressed recombinant product. Once the vector has been incorporated into the appropriate host, the host is maintained under conditions suitable for high level expression of the nucleotide sequences, and, as desired, the collection and purification of the proteins, antigenic fragments or fusion proteins of the invention may follow. Of course, the vector can also comprise regulatory regions from pathogenic organisms.

The present invention in addition relates to a host transformed with a vector of the present invention or to a host comprising the nucleic acid molecule of the invention. Said host may be produced by introducing said vector or nucleotide sequence into a host cell which upon its presence in the cell mediates the expression of a protein encoded by the nucleotide sequence of the invention or comprising a nucleotide sequence or a vector according to the invention wherein the nucleotide sequence and/or the encoded polypeptide is foreign to the host cell.

By “foreign” it is meant that the nucleotide sequence and/or the encoded polypeptide is either heterologous with respect to the host, this means derived from a cell or organism with a different genomic background, or is homologous with respect to the host but located in a different genomic environment than the naturally occurring counterpart of said nucleotide sequence. This means that, if the nucleotide sequence is homologous with respect to the host, it is not located in its natural location in the genome of said host, in particular it is surrounded by different genes. In this case the nucleotide sequence may be either under the control of its own promoter or under the control of a heterologous promoter. The location of the introduced nucleic acid molecule or the vector can be determined by the skilled person by using methods well-known to the person skilled in the art, e.g., Southern Blotting. The vector or nucleotide sequence according to the invention which is present in the host may either be integrated into the genome of the host or it may be maintained in some form extrachromosomally. In this respect, it is also to be understood that the nucleotide sequence of the invention can be used to restore or create a mutant gene via homologous recombination.

Said host may be any prokaryotic or eukaryotic cell. Suitable prokaryotic/bacterial cells are those generally used for cloning like E. coli, Salmonella typhimurium, Serratia marcescens or Bacillus subtilis. Said eukaryotic host may be a mammalian cell, an amphibian cell, a fish cell, an insect cell, a fungal cell, a plant cell or a bacterial cell (e.g., E coli strains HB101, DH5a, XL1 Blue, Y1090 and JM101). Eukaryotic recombinant host cells are preferred. Examples of eukaryotic host cells include, but are not limited to, yeast, e.g., Saccharomyces cerevisiae, Schizosaccharomyces pombe, Kluyveromyces lactis or Pichia pastoris cells, cell lines of human, bovine, porcine, monkey, and rodent origin, as well as insect cells, including but not limited to, Spodoptera frugiperda insect cells and Drosophila-derived insect cells as well as zebra fish cells. Mammalian species-derived cell lines suitable for use and commercially available include, but are not limited to, L cells, CV-1 cells, COS-1 cells (ATCC CRL 1650), COS-7 cells (ATCC CRL 1651), HeLa cells (ATCC CCL 2), C1271 (ATCC CRL 1616), BS-C-1 (ATCC CCL 26) and MRC-5 (ATCC CCL 171).

In a more preferred embodiment, the host according to the invention is a non-human transgenic organism. Said non-human organism may be a mammal, an amphibian, a fish, an insect, a fungus or a plant. Particularly preferred non-human transgenic animals are Drosophila species, Caenorhabditis elegans, Xenopus species, zebra fish, Spodoptera frugiperda, Autographa californica, mice and rats. Transgenic plants comprise, but are not limited to, wheat, tobacco, parsley and Arabidopsis. Transgenic fungi are also well known in the art and comprise, inter alia, yeasts, like S. pombe or S. cerevisae, or Aspergillus spec, Neurospora or Ustilago species or Pichia species.

In another embodiment, the present invention relates to a method for producing the polypeptide encoded by a nucleic acid molecule of the invention comprising culturing/raising the host of the invention and isolating the produced polypeptide.

A large number of suitable methods exist in the art to produce polypeptides in appropriate hosts. If the host is a unicellular organism or a mammalian or insect cell, the person skilled in the art can revert to a variety of culture conditions that can be further optimized without an undue burden of work. Conveniently, the produced protein is harvested from the culture medium or from isolated (biological) membranes by established techniques. Furthermore, the produced polypeptide may be directly isolated from the host cell. Said host cell may be part of or derived from a part of a host organism. Additionally, the produced polypeptide may be isolated from fluids derived from said host.

The polypeptide of the invention and the polypeptide (enzyme) to be employed in the screening methods of the invention may accordingly be produced by microbiological methods or by transgenic mammals. It is also envisaged that the polypeptide of the invention is recovered from transgenic plants. Alternatively, the polypeptide of the invention may be produced synthetically or semi-synthetically.

For example, chemical synthesis, such as the solid phase procedure described by Houghton Proc. Natl. Acad. Sci. USA (82) (1985), 5131-5135, can be used. Another method is in vitro translation of mRNA. A preferred method involves the recombinant production of protein in host cells as described above. For example, nucleotide acid sequences comprising all or a portion of any one of the nucleotide sequences according to the invention can be synthesized by PCR, inserted into an expression vector, and a host cell transformed with the expression vector. Thereafter, the host cell is cultured to produce the desired polypeptide, which is isolated and purified. Protein isolation and purification can be achieved by any one of several known techniques; for example and without limitation, ion exchange chromatography, gel filtration chromatography and affinity chromatography, high pressure liquid chromatography (HPLC), reversed phase HPLC, preparative disc gel electrophoresis. In addition, cell-free translation systems can be used to produce the polypeptides of the present invention. Suitable cell-free expression systems for use in accordance with the present invention include rabbit reticulocyte lysate, wheat germ extract, canine pancreatic microsomal membranes, E. coli S30 extract, and coupled transcription/translation systems such as the TNT-system (Promega). These systems allow the expression of recombinant polypeptides or peptides upon the addition of cloning vectors, DNA fragments, or RNA sequences containing coding regions and appropriate promoter elements. As mentioned supra, protein isolation/purification techniques may require modification of the proteins of the present invention using conventional methods. For example, a histidine tag can be added to the protein to allow purification on a nickel column (IMAC). Other modifications may cause higher or lower activity, permit higher levels of protein production, or simplify purification of the protein.

The enzymes provided herein and involved in the fungal siderophore biosynthesis are particularly useful (in form of expressed enzymes (or fragments thereof) as well as in form of the polynucleotides as provided herein (in form of coding sequences as well as non-coding sequences)) in the methods for screening inhibitors of fungal siderophore biosynthesis of the present invention.

Furthermore an antibody specifically binding to the polypeptide having L-ornithine N⁵-oxygenase, N5-transacylase, non-ribosomal peptide synthetase, enoyl CoA hydratase, or N²-transacetylase activity is within the scope of the present invention. Moreover, an antibody specifically binding to other elements involved in siderophore biosynthesis is also contemplated, for example, an antibody specifically binding to elements involved in the secretion of fungal siderophores from the intracellular to the extracellular milieu, such as transporters, channels or the like or an antibody specifically binding to elements involved in the uptake of siderophores from the extracellular milieu or an antibody specifically binding to elements involved in the uncoupling or detaching of iron or elements involved in channeling in iron into the metabolism of a fungal cell as described hereinabove. The antibodies are also useful as inhibitors of siderophore biosynthesis.

In another aspect the present invention relates to an antibody or aptamer specifically recognizing a fungal siderophore(s) which is/are described herein. Aptamers commonly comprise RNA, single stranded DNA, modified RNA or modified DNA molecules. The preparation of aptamers is well known in the art and may involve, inter alia, the use of combinatorial RNA libraries to identify binding sides (Gold, Ann. Rev. Biochem. 64 (1995), 763-797).

The term “specifically” in this context means that the antibody reacts with the elements involved in fungal siderophore biosynthesis, such as the polypeptides of the present invention encoded by the polynucleotides of the present invention. Preferably this term also means that such an antibody does not bind to other polypeptides which, may be, related to said polypeptides of the present invention. Whether the antibody specifically reacts as defined herein above can easily be tested, inter alia, by methods known in the art to determine the specificity of an antibody, such as ELISA, etc.

The antibody of the present invention can be, for example, polyclonal or monoclonal. The term “antibody” also comprises derivatives or fragments thereof which still retain the binding specificity. Techniques for the production of antibodies are well known in the art and described, e.g. in Harlow and Lane “Antibodies, A Laboratory Manual”, CSH Press, Cold Spring Harbor, 1988. These antibodies can be used, for example, for the immunoprecipitation and immunolocalization of the polypeptides of the invention as well as for the monitoring of the presence of such polypeptides, for example, in recombinant organisms or in diagnosis. They can also be used for the identification of compounds interacting with the proteins according to the invention (as mentioned herein below). For example, surface plasmon resonance as employed in the BIAcore system can be used to increase the efficiency of phage antibodies which bind to an epitope of the polypeptide of the invention (Schier, Human Antibodies Hybridomas 7 (1996), 97-105; Malmborg, J. Immunol. Methods 183 (1995), 7-13).

The present invention furthermore includes chimeric, single chain and humanized antibodies, as well as antibody fragments, like, inter alia, Fab fragments. Antibody fragments or derivatives further comprise F(ab′)2, Fv or scFv fragments; see, for example, Harlow and Lane, loc. cit. Various procedures are known in the art and may be used for the production of such antibodies and/or fragments. Thus, the (antibody) derivatives can be produced by peptidomimetics. Further, techniques described for the production of single chain antibodies (see, inter alia, U.S. Pat. No. 4,946,778) can be adapted to produce single chain antibodies to polypeptide(s) of this invention. Also, transgenic animals may be used to express humanized antibodies to polypeptides of this invention, i.e. polypeptides involved in the siderophore biosynthesis. Most preferably, the antibody of this invention is a monoclonal antibody. For the preparation of monoclonal antibodies, any technique which provides antibodies produced by continuous cell line cultures can be used. Examples for such techniques include the hybridoma technique (Köhler and Milstein Nature 256 (1975), 495-497), the trioma technique, the human B-cell hybridoma technique (kozbor, Immunology Today 4 (1983), 72) and the EBV-hybridoma technique to produce, human monoclonal antibodies (Cole et al., Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to immunogenic polypeptides as described above. Furthermore, transgenic mice may be used to express humanized antibodies directed against said immunogenic polypeptides. It is in particular preferred that the antibodies/antibody constructs as well as antibody fragments or derivatives to be employed in accordance with this invention or capable to be expressed in a cell. This may, inter alia, be achieved by direct injection of the corresponding proteineous molecules or by injection of nucleic acid molecules encoding the same. Furthermore, gene therapy approaches are envisaged. Accordingly, in context of the present invention, the term “antibody molecule” relates to full immunoglobulin molecules as well as to parts of such immunoglobulin molecules. Furthermore, the term relates, as discussed above, to modified and/or altered antibody molecules, like chimeric and humanized antibodies. The term also relates to monoclonal or polyclonal antibodies as well as to recombinantly or synthetically generated/synthesized antibodies. The term also relates to intact antibodies as well as to antibody fragments thereof, like, separated light and heavy chains, Fab, Fab/c, Fv, Fab′, F(ab′)2. The term “antibody molecule” also comprises bifunctional antibodies and antibody constructs, like single chain Fvs (scFv) or antibody-fusion proteins. It is also envisaged in context of this invention that the term “antibody” comprises antibody constructs which may be expressed in cells, e.g. antibody constructs which may be transfected and/or transduced via, inter alia, viruses or vectors. It is also envisaged in context of this invention that the term “antibody” comprises antibody constructs which may be expressed in cells, e.g. antibody constructs which may be transfected and/or transduced via, inter alia, viruses or vectors. It is particularly envisaged that such antibody constructs specifically recognize the elements involved in siderophore biosynthesis as described herein, such as the polypeptides of the present invention. It is moreover envisaged that such an antibody specifically recognizes (a) fungal siderophore(s) as is described herein. Accordingly, it is, furthermore, envisaged that said antibody construct is employed in gene therapy approaches for treating and/or preventing the diseases associated with fungal infection which are described herein. Therefore, not only the antibodies provided herein and directed against the herein identified genes of the siderophore synthesis may be medically used, but also nucleic acid molecules encoding the same.

For gene therapy (for example expressing antisense molecules, ribozymes, siRNAs and the like directed against target sequences of the siderophore biosynthesis enzymes provided herein), various viral vectors which can be utilized, for example, adenovirus, herpes virus, vaccinia, or, preferably, an RNA virus such as a retrovirus.

Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can also incorporate multiple genes. All of these vectors can transfer or incorporate a gene for a selectable marker so that transduced cells can be identified and generated.

Retroviral vectors can be made target specific by inserting, for example, a polynucleotide encoding a sugar, a glycolipid, or a protein. Those of skill in the art will know of, or can readily ascertain without undue experimentation, specific polynucleotide sequences, for example polynucleotide sequences encoding an antibody of the present invention, which can be inserted into the retroviral genome to allow target specific delivery of the retroviral vector containing the inserted polynucleotide sequence.

Since recombinant retroviruses are preferably defective, they require assistance in order to produce infectious viral particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include, but are not limited to w2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced. Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium. Another targeted delivery system for polynucleotides encoding an antibody of the present invention is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 pm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

In the context of the present invention it is contemplated that the siderophore produced by a fungal species, preferably an Aspergillus species, more preferably Aspergillus fumigatus as described herein is an extracellular siderophore. Extracellular siderophores are known from other fungal species, such as rhodotorulic acid, ferrichrome, ferrichrome A, fusarinine C, triacetylfusarinine C or coprogen. It is envisaged that the extracellular siderophore of Aspergillus fumigatus is similar to fusarinine C or triacetylfusarinine C of Aspergillus nidulans.

Yet, it is also contemplated in the context of the present invention that the siderophore produced by a fungal species, preferably an Aspergillus species, more preferably Aspergillus fumigatus as described herein is an intracellular siderophore. Intracellular or cellular siderophores are known from fungi, such as ferrichrome or ferricrocin. It is envisaged that the cellular siderophore of Aspergillus fumigatus is similar to ferricrocin of Aspergillus nidulans. An explanation for the crucial role of siderophores in the pathogenicity of Aspergillus fumigatus may be that intracellular siderophores such as ferricrocin are involved in defense against oxidative stress.

Accordingly, biosynthesis of intracellular siderophores might be—alternatively or in combination—important in the defense against the host's immune system because it has been shown that killing of A. fumigatus by alveolar macrophages is mediated by reactive oxidant intermediates (Philippe (2003), Infect. Immun. 71, 3034-3042).

The structure of extracellular and intracellular (i.e. cellular) siderophores is described in detail in Haas (2003), Appl. Microbiol Biotechnol 62, 316-330. In brief, there are four major families of fungal hydroxamate-type siderophores, i.e. rhodotorulic acid, fusarinines, coprogens and ferrichromes. In all these fungal siderophores, the nitrogen of the hydroxamate group is derived from N⁵-hydroxyornithine. Completion of the hydroxamate prosthetic group requires N⁵-acylation, e.g., acetyl, anhydromevalonyl or methylglutaconyl. Most siderophores contain three hydroxamate groups linked by peptide or ester bonds to form an octahedral complex. The simplest structure, rhodotorulic acid, is the diketopiperazine of N⁵-acetyl-N⁵-hydroxyornithine. Importantly, this siderophore contains only two hydroxamate groups and forms Fe₂(rhodotorulic acid)₃ complexes. The prototype of fusarinines, the cyclic fusarine C (or fusigen), consists of three N⁵-cis-anhydromevalonyl-N⁵-hydroxyornithines (termed cis-fusarinine), linked by ester bonds. N²-acetylation of fusarine C results in the more stable triacetylfusarinine C. Coprogens contain two trans-fusarinine moieties connected head-to-head by a peptide bond to form a diketopiperazine unit (dimerium acid) and a third trans-fusarinine molecule esterified to the C-terminal group of dimerium acid. Ferrichromes like ferrichrome, ferrichrome A and ferricrocin are cyclic hexapeptides consisting of three N′-acyl-N⁵-hydroxyornithines and three amino acids-combinations of glycine, serine or alanine. It is important to note that “ferrichrome” and “coprogen” refer to specific members of their respective families.

The first committed step in siderophore biosynthesis is the N⁵-hydroxylation of ornithine catalyzed by ornithine N⁵-oxygenase. The formation of the hydroxamate group is conducted by the transfer of an acyl group from acyl-CoA derivatives to N⁵-hydroxyornithine. Linking of the hydroxamate groups and, in the case of ferrichromes, additional incorporation of a further three amino acids, is carried out by nonribosomal peptide synthetases. These proteins are exceptionally large multifunctional enzymes with a modular construction able to assemble compounds from a remarkable range of proteinogenic and nonproteinogenic precursors. Each module contains an adenylation domain, a condensation domain and a peptidyl carrier. As the acyl carrier domain in fatty acid and polyketide synthases, the peptidyl carrier domain contains phosphopantetheine as a covalently linked cofactor, which is attached by 4′-phosphopantetheine transferase. Nonribosomal peptide synthetases are able to form peptide and ester bonds; the peptidyl chain grows directionally in incremental steps and, for cyclic products, the final condensation must lead to ring closure.

Another preferred embodiment of the present invention envisages that the compound to be tested for its capability to inhibit fungal siderophore biosynthesis is of chemical or biological origin as is described in detail hereinabove.

The present invention envisages in a furthermore preferred embodiment that compound to be tested for its capability to inhibit fungal siderophore biosynthesis is synthetically, recombinantly and/or chemically produced as is described in detail hereinabove.

Moreover, in a preferred embodiment the method for screening inhibitors of fungal siderophore biosynthesis are screened in a high through put screening assay. High-throughput screening methods are described in U.S. Pat. Nos. 5,585,277 and 5,679,582, in U.S. Ser. No. 08/547,889, and in the published PCT application PCT/US96/19698, and may be used for identifying an inhibitor of fungal siderophore biosynthesis as described herein. High-throughput screening methods and similar approaches which are known in the art (Spencer, Biotechnol. Bioeng. 61 (1998), 61-67; Oldenburg, Annu. Rev. Med. Chem. 33 (1998), 301-311) carried out using 96-well, 384-well, 1536-well (and other) commercially available plates. In this method, large numbers of different small test compounds, e.g. aptamers, peptides, low-molecular weight compounds as described herein, are provided or synthesized on a solid substrate, such as plastic pins or some other surface. Further methods to be employed in accordance with the present invention comprise, but are not limited to, homogenous fluorescence readouts in high-throughput screenings (as described, inter alia, in Pope, Drug Discovery Today 4 (1999), 350-362).

The test compounds are reacted either with a cell expressing a fungal siderophore or with enzymes either in purified, partially purified or unpurified form, such as whole cell extracts, involved in the siderophore biosynthesis of the fungi described herein. In particular, said enzymes are selected from the group consisting of L-ornithine N⁵-oxygenase, N⁵-transacylase, non-ribosomal peptide synthetase, enoyl CoA hydratase and N²-transacetylase and/or fragments thereof. It is to be understood that also combinations of the aforementioned enzymes may be used in a high through put assay.

Usually, various predetermined concentrations of test compounds are used for screening, such as 0.01 micromolar, 1 micromolar and 10 micromolar. Test compound controls can include the measurement of a signal in the absence of the test compound or comparison to a compound known to inhibit the target.

In a preferred embodiment, the individual sample incubation volumes are less than about 500 μl, preferably less than about 250 μl, more preferably less than about 100 μl. Such small sample volumes minimize the use of often scarce candidate agent, expensive enzymes, and hazardous radioactive waste. Furthermore, the methods provide for automation, especially computerized automation. Accordingly, the method steps are preferably performed by a computer-controlled electromechanical robot.

While individual steps may be separately automated, a preferred embodiment provides a single computer-controlled multifunction robot with a single arm axially rotating to and from a plurality of work stations performing the mixture forming, incubating and separating steps. The computer is loaded with software which provides the instructions which direct the arm and work station operations and provides input (e.g. keyboard and/or mouse) and display (e.g. monitor) means for operator interfacing. In a particular embodiment, the robotic arm is equipped with a general purpose retrieving hand and a pipetting hand. The pipetting hand equipped with a multichannel pipettor retrieves and transfers measured aliquots of each an assay buffer, a solution comprising one or more candidate agents as described herein. The general purpose hand then transfers each microtiter plate to the next stage of the automated high through put device.

In addition to the high-throughput screening techniques described above, technologies for molecular identification can be employed in the identification of inhibitor molecules. One of these technologies is phage display technology (U.S. Pat. No. 5,403,484. Viruses Expressing Chimeric Binding Proteins).

Another relatively new screening technology which may be applied to the inhibitor screening assays of this invention is biospecific interaction analysis (BIAcore, Pharmacia Biosensor AB, Uppsala, Sweden). This technology is described in detail by Jonsson in Biotechniques 11:5, 620-627 (1991)). Biospecific interaction analysis utilizes surface plasmon resonance (SPR) to monitor the adsorption of biomolecular complexes on a sensor chip. SPR measures the changes in refractive index of a polarized light directed at the surface of the sensor chip. Specific ligands (i.e., candidate inhibitors) capable of binding to the target molecule of interest are immobilized to the sensor chip. In the presence of the target molecule, specific binding to the immobilized ligand occurs. The nascent immobilized ligand-target molecule complex causes a change in the refractive index of the polarized light and is detected on a diode array. Biospecific interaction analysis provides the advantages of; 1) allowing for label-free studies of molecular complex formation; 2) studying molecular interactions in real time as the assay is passed over the sensor chip; 3) detecting surface concentrations down to 10 pg/mm; detecting interactions between two or more molecules; and 4) being fully automated.

Once a putative inhibitor has been identified in the primary screen or screens of the present invention, it may be desirable to determine the effect of the inhibitor on the growth and/or viability of the fungal species described herein, in particular Aspergillus fumigatus, in culture. Methods for performing tests on fungal growth inhibition in culture are well-known in the art. Non-limiting examples of such procedures test the candidate inhibitor compounds for antifungal activity against a panel of Aspergillus strains: One such procedure is based on the NCCLS M27A method (The National Committee for Clinical Laboratory Standards, Reference Method for Broth Microdilution Antifungal Susceptibility Testing of Yeasts; approved standard, 1997) to measure minimum inhibitory concentrations (MICs) and minimum fungicidal concentrations (MFCs).

When performing the methods of the present invention it is preferred that the cell which is contacted with a compound to be tested is selected from the group consisting of an animal cell, e.g., a mammalian cell, insect cell, amphibian cell or fish cell, a plant cell, a fungal cell and bacterial cell as described herein.

Preferably, the cell which is contacted with a compound to be tested harbours one or more polynucleotides operatively linked to expression control sequences capable of expressing one or more of the enzymes involved in siderophore biosynthesis as described herein. It is thus to be understood that said cell is capable of synthesizing the fungal siderophores as described herein, preferably said cell produces the siderophore of Aspergillus spec., particularly preferably the siderophore of Aspergillus fumigatus. As pointed out above, it is also envisaged that the cell to be employed in screening assay comprises and expresses a herein identified enzyme. Said enzyme may be expressed heterologously. It is also envisaged that not any one but more enzymes as defined herein are expressed in said (host) cell.

Another aspect of the present invention is a method for the production of a pharmaceutical composition comprising the steps of the method for screening inhibitors of fungal siderophore biosynthesis, which preferably takes place in Aspergillus species, more preferably in Aspergillus fumigatus, and the subsequent step of mixing the compound identified to be an inhibitor of fungal siderophore biosynthesis with a pharmaceutically acceptable carrier. Yet, also inhibitors identified by the in vitro methods provided herein (enzymatic assays, gene expression or gene regulation assays, christallographic methods, spectroscopical methods, magnetic resonance spectroscopy, X-ray analysis/christallography, and the like) may be mixed with a pharmaceutically acceptable carrier.

A furthermore aspect of the present invention relates to a method for preparing a pharmaceutical composition for treating diseases associated with fungal infections, particularly, aspergillosis or coccidiosis comprising (a) identifying a compound which inhibits fungal siderophore biosynthesis; and (b) formulating said compound with a pharmaceutically acceptable carrier. In a preferred embodiment, said identifying step is performed by the methods for screening a fungal siderophore biosynthesis inhibitor in accordance with the present invention.

Furthermore the present invention relates to a pharmaceutical composition comprising an inhibitor of siderophore biosynthesis in Aspergillus species, particularly in Aspergillus fumigatus.

Potential inhibitor(s) or partial inhibitors(s) for fungal siderophore biosynthesis may be selected from aptamers (Gold, Ann. Rev. Biochem. 64 (1995), 763-797)), aptazymes, RNAi, shRNA, RNAzymes, ribozymes (see e.g., EP-B1 0 291 533, EP-A1 0 321 201, EP-B1 0 360 257), antisense DNA, antisense oligonucleotides, antisense RNA, si RNA, antibodies (Harlow and Lane “Antibodies, A Laboratory Manual”, CSH Press, Cold Spring Harbor, 1988), affibodies (Hansson, Immunotechnology 4 (1999), 237-252; Henning, Hum Gene Ther. 13 (2000), 1427-1439), trinectins (Phylos Inc., Lexington, Mass., USA; Xu, Chem. Biol. 9 (2002), 933), anticalins, or the like. These compounds are, for example, described in EP 1 017 814. Said European patent also describes the process of preparing such anticalins with the ability to bind a specific target. Other potential inhibitors which can be used as a pharmaceutical composition are identified by the methods as described herein.

In accordance with the present invention, the term “aptamer” means nucleic acid molecules that can bind to target molecules. Aptamers commonly comprise RNA, single stranded DNA, modified RNA or modified DNA molecules. The preparation of aptamers is well known in the art and may involve, inter alia, the use of combinatorial RNA libraries to identify binding sites (Gold (1995), Ann. Rev. Biochem 64, 763-797).

Antisense technology can be used to control gene expression through triple-helix formation or antisense DNA or RNA, whereby the inhibitory effect is based on specific binding of a nucleic acid molecule to DNA or RNA. For example, the 5′ coding portion of a nucleic acid molecule encoding an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof to be inhibited can be used to design an antisense oligonucleotide, e.g., of at least 10 nucleotides in length. The antisense DNA or RNA oligonucleotide hybridises to the mRNA in vivo and blocks translation of said mRNA and/or leads to destabilization of the mRNA molecule (Okano, J. Neurochem. 56 (1991), 560; Oligodeoxynucleotides as antisense inhibitors of gene expression, CRC Press, Boca Raton, Fla., USA (1988). Recently, an anitsense approach has been employed in Aspergillus species (Ngiam (2000), Appl Environ Microbiol. 66, 775-82; Bautista (2000), Appl Environ Microbiol. 66, 4579-4581; Juwadi. (2003), Arch Microbiol. 179, 416-422).

The antisense molecule may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-mannosylqueosine, 5′-methoxycarboxymethyluracil, 5-methoxyuracil, 2-methylthio-N6 isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil 5-oxyacetic acid methylester, uracil-5-oxyacetic acid (v), 5-methyl-2-thiouracil, 3-(3-amin3-N-2-carboxypropyl)uracil, and 2,6-diaminopurine. The antisense molecule may also comprise at least one modified sugar moiety selected from the group including but not limited to arabinose, 2-fluoroarabinose, xylulose, and hexose. In yet another embodiment, the antisense molecule comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense molecule is an a-anomeric oligonucleotide. An a-anomeric oligonucleotide forms specific double-stranded hybrids with complementary RNA in which, contrary to the usual-units, the strands run parallel to each other (Gautier, 1987, Nucl. Acids Res. 15: 6625-6641). The oligonucleotide is a 2′-O-methylribonucleotide (Inoue, 1987, Nucl. Acids Res. 15: 6131-6148), or a chimeric RNA-DNA analogue (Inoue, 1987, FEBS Lett. 215: 327-330).

Antisense molecules of the invention (and to be employed as “inhibitors” of the siderophore biosynthesis) may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein (1988, Nucl. Acids Res. 16:3209), methylphosphonate oligonucleotides can be prepared by use of controlled pore glass polymer supports (Sarin, 1988, Proc. Natl. Acad. Sci. U.S.A. 85: 7448-7451), etc.

For applying a triple-helix approach, a DNA oligonucleotide can be designed to be complementary to a region of the gene encoding an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof to be inhibited according to the principles laid down in the prior art (see for example Lee, Nucl. Acids Res. 6 (1979), 3073; Cooney, Science 241 (1988), 456; and Dervan, Science 251 (1991), 1360). Such a triple helix forming oligonucleotide can then be used to prevent transcription of the specific gene, and is, accordingly, an inhibition in the sense of this invention. The oligonucleotides described above can also be delivered to target cells via a gene delivery vector as described above in order to express such molecules in vivo to inhibit gene expression of the respective protein.

Examples for antisense molecules are oligonucleotides specifically hybridising to a polynucleotide encoding an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof. Such oligonucleotides have a length of preferably at least 10, in particular at least 15, and particularly preferably of at least 50 nucleotides. They are characterized in that they specifically hybridise to said polynucleotide, that is to say that they do not or only to a very minor extent hybridise to other nucleic acid sequences.

Another suitable approach is the use of nucleic acid molecules mediating an RNA interference (RNAi) effect. RNAi refers to the introduction of homologous double stranded RNA (dsRNA) to specifically target a gene's product, resulting in null or hypomorphic phenotypes. Introduction of dsRNA into a fungal cell results in the loss of the function of an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof. Because RNAi is also remarkably potent (i.e., only a few dsRNA molecules per cell are required to produce effective interference), the dsRNA must be either replicated and/or work catalytically. Thereby, the formation of double-stranded RNA leads to an inhibition of gene expression in a sequence-specific fashion. More specifically, in RNAi constructs, a sense portion comprising the coding region of the gene to be inactivated (or a part thereof, with or without non-translated region) is followed by a corresponding antisense sequence portion. Between both portions, an intron not necessarily originating from the same gene may be inserted. After transcription, RNAi constructs form typical hairpin structures. In accordance with the method of the present invention, the RNAi technique may be carried out as described by Smith (Nature 407 (2000), 319-320) or Marx (Science 288 (2000), 1370-1372). Recently, an RNAi approach has been employed in Aspergillus species (Yin, J Biol. Chem. (2003), 52454-52460) Likewise, RNA molecules with ribozyme activity which specifically cleave transcripts of a gene encoding an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof can be used. Said ribozymes may also target DNA molecules encoding the corresponding RNAs. Ribozymes are catalytically active RNA molecules capable of cleaving RNA molecules and specific target sequences. By means of recombinant DNA techniques it is possible to alter the specificity of ribozymes. There are various classes of ribozymes. For practical applications aiming at the specific cleavage of the transcript of a certain gene, use is preferably made of representatives of two different groups of ribozymes. The first group is made up of ribozymes which belong to the group I intron ribozyme type. The second group consists of ribozymes which as a characteristic structural feature exhibit the so-called “hammerhead” motif. The specific recognition of the target RNA molecule may be modified by altering the sequences flanking this motif. By base pairing with sequences in the target molecule these sequences determine the position at which the catalytic reaction and therefore the cleavage of the target molecule takes place. Since the sequence requirements for an efficient cleavage are low, it is in principle possible to develop specific ribozymes for practically each desired RNA molecule. In order to produce DNA molecules encoding a ribozyme which specifically cleaves transcripts of a gene encoding an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof, for example a DNA sequence encoding a catalytic domain of a ribozyme is bilaterally linked with DNA sequences which are homologous to sequences encoding the target protein. Sequences encoding a catalytic domain and DNA sequence flanking the catalytic domain are preferably derived from the polynucleotides encoding an enzyme involved in fungal siderophore biosynthesis, preferably at least selected from the group consisting of L-ornithine N⁵-oxygenase, N⁵-transacylase, non-ribosomal peptide synthetase, enoyl CoA hydratase and N²-transacetylase and/or fragments thereof. The expression of ribozymes in order to decrease the activity in certain proteins is also known to the person skilled in the art and is, for example, described in EP-B1 0 321 201 or EP-B1 0 360 257.

In another preferred embodiment, the inhibiting nucleic acid molecule is siRNA as dislosed in Elbashir (2001), Nature 411, 494-498.

The shRNA approach for gene silencing is well known in the art and may comprise the use of st (small temporal) RNAs; see, inter alia, Paddison (2002) Genes Dev. 16, 948-958. As mentioned above, approaches for gene silencing are known in the art and comprise “RNA”-approaches like RNAi or siRNA. Successful use of such approaches has been shown in Paddison (2002) loc. cit., Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA pol III vectors may be employed as illustrated, inter alia, in Yu (2002) loc. cit.; Miyagishi (2002) loc. cit. or Brummelkamp (2002) loc. cit.

It is envisaged that said siRNA is targeted to deplete enzymes selected from the group consisting of L-ornithine N⁵-oxygenase, N⁵-transacylase, non-ribosomal peptide synthetase, enoyl CoA hydratase and N²-transacetylase and/or fragments thereof. In accordance with the present invention the term “targeted” means that (an) siRNA duplex(es) is/are specifically targeted to a coding sequence of enzymes selected from the group consisting of L-ornithine N⁵-oxygenase, N⁵-transacylase, non-ribosomal peptide synthetase, enoyl CoA hydratase and N²-transacetylase and/or fragments thereof, to cause gene silencing by RNA interference (RNAi) since said siRNA duplex(es) is/are homologous in sequence to a gene desired to be silenced, for example, an enzyme selected from the group consisting of L-ornithine N⁵-oxygenase, N⁵-transacylase, non-ribosomal peptide synthetase, enoyl CoA hydratase and N²-transacetylase and/or fragments thereof. “Homologous in sequence” in the context of the present invention means that said siRNA duplex(es) is/are homologous in the sequence to a gene, for example the L-ornithine N⁵-oxygenase gene, N⁵-transacylase gene, non-ribosomal peptide synthetase gene, enoyl CoA hydratase or N²-transacetylase gene or fragments thereof desired to be silenced by the mechanism/pathway of RNA interference (RNAi). It is envisaged that the degree of homology between the siRNA duplex(es) and the sequence of the gene desired to be silenced is sufficient that said siRNA duplex(es) is/are capable to cause gene silencing of said desired gene initiated by double-stranded RNA (dsRNA), for example, (an) siRNA duplex(es). The person skilled in the art is readily in a position to determine whether the degree of homology is sufficient to deplete an enzyme selected from the group consisting of L-ornithine N⁵-oxygenase, N⁵-transacylase, non-ribosomal peptide synthetase, enoyl CoA hydratase and N²-transacetylase and/or fragments thereof.

siRNAs are extremely potent therapeutic tools as recently illustrated in Soutschek (2004) Nature 432, 173-178.

The following table provides for exemplified target sequences which may be targeted by inhibitors of the present invention. The table also provides for selected useful “strand” and “antistrand” RNAs which are particularly useful as siRNA to inhibit said target sequences and/or their expression. Accordingly, the following sequences provide (in form of “sense” and “antisense strands”) siRNA duplex(es) particularly useful in the medical intervention of a fungal infection.

For sidA the targets and corresponding, exemplified siRNAs are:

| | |
|---|---|
| Target sequence 1: | AAGTCGAAGCCTTACAACATT |
| GC content: | 38.1% |
| Sense strand siRNA: | GUCGAAGCCUUACAACAUUtt |
| Antisense strand siRNA: | AAUGUUGUAAGGCUUCGACtt |
| Target sequence 2: | AACAAGTCCGCTTCCAATATC |
| GC content: | 42.9% |
| Sense strand siRNA: | CAAGUCCGCUUCCAAUAUCtt |
| Antisense strand siRNA: | GAUAUUGGAAGCGGACUUGtt |
| Target sequence 3: | AAGTCCGCTTCCAATATCCAT |
| GC content: | 42.9% |
| Sense strand siRNA: | GUCCGCUUCCAAUAUCCAUtt |
| Antisense strand siRNA: | AUGGAUAUUGGAAGCGGACtt |
| Target sequence 4: | AAGGACAAGTCGAAGCCTTAC |
| GC content: | 47.6% |
| Sense strand siRNA: | GGACAAGUCGAAGCCUUACtt |
| Antisense strand siRNA: | GUAAGGCUUCGACUUGUCCtt |
| Target sequence 5: | AACAACGCTGATTATGCGGGA |
| GC content: | 47.6% |
| Sense strand siRNA: | CAACGCUGAUUAUGCGGGAtt |
| Antisense strand siRNA: | UCCCGCAUAAUCAGCGUUGtt |
| Target sequence 6: | AAGGCGCAGCAAACGACGTCA |
| GC content: | 57.1% |
| Sense strand siRNA: | GGCGCAGCAAACGACGUCAtt |
| Antisense strand siRNA: | UGACGUCGUUUGCUGCGCCtt |

For sidD the targets and corresponding, exemplified siRNAs are:

| | |
|---|---|
| Target sequence 1: | AACCTGCCTCCCACGTACAAT |
| GC content: | 52.4% |
| Sense strand siRNA: | CCUGCCUCCCACGUACAAUtt |
| Antisense strand siRNA: | AUUGUACGUGGGAGGCAGGtt |
| Target sequence 2: | AAGGGTTACTTCACCTAGAAA |
| GC content: | 38.1% |
| Sense strand siRNA: | GGGUUACUUCACCUAGAAAtt |
| Antisense strand siRNA: | UUUCUAGGUGAAGUAACCCtt |
| Target sequence 3: | AAAGCATTGTTGGACCATTAA |
| GC content: | 33.3% |
| Sense strand siRNA: | AGCAUUGUUGGACCAUUAAtt |
| Antisense strand siRNA: | UUAAUGGUCCAACAAUGCUtt |
| Target sequence 4: | AAGCCGTGCAGCAGAGTGTTT |
| GC content: | 52.4% |
| Sense strand siRNA: | GCCGUGCAGCAGAGUGUUUtt |
| Antisense strand siRNA: | AAACACUCUGCUGCACGGCtt |
| Target sequence 5: | AACCTGGCGACGGAGATCATA |
| GC content: | 52.4% |
| Sense strand siRNA: | CCUGGCGACGGAGAUCAUAtt |
| Antisense strand siRNA: | UAUGAUCUCCGUCGCCAGGtt |
| Target sequence 6: | AAACCTACACCAGTAGCGCCA |
| GC content: | 52.4% |
| Sense strand siRNA: | ACCUACACCAGUAGCGCCAtt |
| Antisense strand siRNA: | UGGCGCUACUGGUGUAGGUtt |

For rac1 the targets and corresponding, exemplified siRNAs are:

| | |
|---|---|
| Target sequence 1: | AACCTGCTCTATATGTGGCCA |
| GC content: | 47.6% |
| Sense strand siRNA: | CCUGCUCUAUAUGUGGCCAtt |
| Antisense strand siRNA: | UGGCCACAUAUAGAGCAGGtt |
| Target sequence 2: | AACCGCTATCTCTAGGAATAG |
| GC content: | 42.9% |
| Sense strand siRNA: | CCGCUAUCUCUAGGAAUAGtt |
| Antisense strand siRNA: | CUAUUCCUAGAGAUAGCGGtt |
| Target sequence 3: | AAGAGGCTAGCAGTGCACTGG |
| GC content: | 57.1% |
| Sense strand siRNA: | GAGGCUAGCAGUGCACUGGtt |
| Antisense strand siRNA: | CCAGUGCACUGCUAGCCUCtt |
| Target sequence 4: | AAGGAATGGAACGACCTCAAC |
| GC content: | 47.6% |
| Sense strand siRNA: | GGAAUGGAACGACCUCAACtt |
| Antisense strand siRNA: | GUUGAGGUCGUUCCAUUCCtt |
| Target sequence 4: | AACGACCTCAACGCGCGTGGT |
| GC content: | 61.9% |
| Sense strand siRNA: | CGACCUCAACGCGCGUGGUtt |
| Antisense strand siRNA: | ACCACGCGCGUUGAGGUCGtt |
| Target sequence 6: | AACGAAATGACAGCCCCCGGG |
| GC content: | 61.9% |
| Sense strand siRNA: | CGAAAUGACAGCCCCCGGGtt |
| Antisense strand siRNA: | CCCGGGGGCUGUCAUUUCGtt |

For at1 the targets and corresponding, exemplified siRNAs are:

| | |
|---|---|
| Target sequence 1: | AAGCGATCGGTCCATGTGTAT |
| GC content: | 47.6% |
| Sense strand siRNA: | GCGAUCGGUCCAUGUGUAUtt |
| Antisense strand siRNA: | AUACACAUGGACCGAUCGCtt |
| Target sequence 2: | AAACCGACACACTCATCCTAC |
| GC content: | 47.6% |
| Sense strand siRNA: | ACCGACACACUCAUCCUACtt |
| Antisense strand siRNA: | GUAGGAUGAGUGUGUCGGUtt |
| Target sequence 3: | AACTACGAGTTCTCCATGAAG |
| GC content: | 42.9% |
| Sense strand siRNA: | CUACGAGUUCUCCAUGAAGtt |
| Antisense strand siRNA: | CUUCAUGGAGAACUCGUAGtt |
| Target sequence 4: | AACGAAGAGCACCTGCAGCTC |
| GC content: | 57.1% |
| Sense strand siRNA: | CGAAGAGCACCUGCAGCUCtt |
| Antisense strand siRNA: | GAGCUGCAGGUGCUCUUCGtt |
| Target sequence 5: | AAGACAAGCATGTCCTGTGTT |
| GC content: | 42.9% |
| Sense strand siRNA: | GACAAGCAUGUCCUGUGUUtt |
| Antisense strand siRNA: | AACACAGGACAUGCUUGUCtt |
| Target sequence 6: | AACCTCCTTCCACTGGACTGG |
| GC content: | 57.1% |
| Sense strand siRNA: | CCUCCUUCCACUGGACUGGtt |
| Antisense strand siRNA: | CCAGUCCAGUGGAAGGAGGtt |

For at2 the targets and corresponding, exemplified siRNAs are:

| | |
|---|---|
| Target sequence 1: | AACTGGGTCTGGCCGAGGTGA |
| GC content: | 61.9% |
| Sense strand siRNA: | CUGGGUCUGGCCGAGGUGAtt |
| Antisense strand siRNA: | UCACCUCGGCCAGACCCAGtt |
| Target sequence 2: | AACGGAGTATGGCTTCCGAGT |
| GC content: | 52.4% |
| Sense strand siRNA: | CGGAGUAUGGCUUCCGAGUtt |
| Antisense strand siRNA: | ACUCGGAAGCCAUACUCCGtt |
| Target sequence 3: | AAGTCGCTGGTTTCCGGCTTT |
| GC content: | 52.4% |
| Sense strand siRNA: | GUCGCUGGUUUCCGGCUUUtt |
| Antisense strand siRNA: | AAAGCCGGAAACCAGCGACtt |
| Target sequence 4: | AAGACGAAGCAATCCAGGTTC |
| GC content: | 47.6% |
| Sense strand siRNA: | GACGAAGCAAUCCAGGUUCtt |
| Antisense strand siRNA: | GAACCUGGAUUGCUUCGUCtt |
| Target sequence 5: | AACCTGGGTCGGCTTTGCCGA |
| GC content: | 61.9% |
| Sense strand siRNA: | CCUGGGUCGGCUUUGCCGAtt |
| Antisense strand siRNA: | UCGGCAAAGCCGACCCAGGtt |
| Target sequence 6: | AAGGCGAGTTCGTGGGTTGGT |
| GC content: | 57.1% |
| Sense strand siRNA: | GGCGAGUUCGUGGGUUGGUtt |
| Antisense strand siRNA: | ACCAACCCACGAACUCGCCtt |

The person skilled in the art can deduce corresponding further “inhibiting molecules” by methods known in the art, like http://www.ambion.com/techlib/misc/siRNA finder.html and http://i.cs.hku.hk/˜sirna/software/sirna.php.

The sequences provided above, in particular the target sequences are also useful in the development of further inhibitors of fungal siderophore biosynthesis, like e.g. antisense molecules, ribozymes, shRNA and the like.

Accordingly, in a preferred embodiment of the invention, the pharmaceutical compositions, uses and therapeutic methods provided herein comprise an inhibitor of fungal siderophore biosynthesis which targets a nucleotide sequence as comprised in any one of SEQ ID NOS: 1, 3, 5, 7, 9 or 16 or which targets an expression product (e.g. RNA or encoded polypeptide/enzyme or fragment thereof) of said sequences. Corresponding sequences are also comprised in SEQ ID NO: 11 which also comprises 5′ untranslated regions which may be targets of the herein described inhibiting molecules. These inhibiting molecules directed against 5′-untranslated regions may be gene regulation/gene expression inhibitors targeting gene regulation sequences and/or promoter sequences. The person skilled in the art is readily in the position to deduce such gene regulation sequences/promoters.

Genes encoding proteins contain gene regulation and/or regions of DNA which are essentially attached to the 5′ terminus of the protein coding region. The promoter regions contain the binding site for RNA polymerase II. RNA polymerase II effectively catalyses the assembly of the messenger RNA complementary to the appropriate DNA strand of the coding region. In most promoter regions, a nucleotide base sequence related to the sequence known generally as a “TATA box” is present and is generally disposed some distance upstream from the start of the coding region and is required for accurate initiation of transcription. Other features important or essential to the proper functioning and control of the coding region are also contained in the promoter region, upstream of the start of the coding region.

Promoters (and/or other gene regulating sequences) may be defined in terms of their abilities to initiate transcription in a suitable test system. An assay for promoter activity uses a quite large DNA fragment of the gene of interest (100 to 500 bp) that is able to initiate transcription e.g. of a reporter enzyme such as luciferase. The boundaries of the sequence constituting the promoter can be determined by reducing the length of the fragment from either end, until at some point it ceases activity in said assay, see inter alia Lewin (1994), Genes V, Oxford University Press.

The method for detecting the activity of the promoter is not particularly limited and includes a method using a reporter gene plasmid carrying the corresponding gene regulation/promoter sequences operatively linked to a marker or label, like an enzyme, a fluorescent label (for example “green fluorescent protein” or luciferase and the like). These are commonly known as “reporter genes”. The reporter gene means a gene encoding a protein which can be assayed by general methods (for example, assay methods known to a person skilled in the art, such as assaying enzyme activity). As such, genes of chloramphenicol acetyltransferase, luciferase, beta-galactosidase and alkaline phosphatase are frequently used, although genes are not limited to those. Concerning the vector as a base for constructing the reporter gene plasmid, there is no limitation. Commercially available plasmid vectors for such as pGV-B2 (manufactured by Toyo Ink) and pSEAP2-Basic (manufactured by Clontech) can be used. The sequence is then inserted in the correct orientation upstream of the reporter genes of these vectors to prepare reporter gene plasmids. The amount of a reporter protein expressed in a cell transformed with such plasmid is assayed by a method appropriate for each of the reporter protein, to determine the presence or absence of the promoter activity of the sequence or the intensity thereof. By adding a test compound to a liquid culture of the transformed cell, the action of the test compound on the gene regulation sequence activity can be analyzed.

The substance inhibiting the activity of the promoter of the present invention highly possibly suppresses or inhibits just some of the physiological functions of enzymes of the fungal siderophore biosynthesis, so that such substance is useful as the active component of an agent for treating fungal infections, like aspergillosis or coccidiosis possibly including long-term pharmaceutical administration. Thus, a cell expressing the promoter of the present invention can be used as a screening tool for the substance inhibiting the activity of the gene regulation sequences of the enzymes of the present invention or an agent for treating and/or preventing fungal infections, in particular aspergillosis or coccidiosis.

The test compounds applicable to the analysis method or screening method of the present invention are not particularly limited and include for example, various known compounds (including peptides) registered in the Chemical File, compound groups obtained by the combinatorial chemistry technique (Terrett, N. K., et al., Tetrahedron, 51, 8135-8137, 1995), or general synthetic techniques, or random peptide groups prepared by the application of the phage display method (Felici, F., et al., J. Mol. Biol., 222, 301-310, 1991). The known compounds described above include for example compounds (including peptides) which have known activities of inhibiting promoters but are still unknown as to whether or not the compounds inhibit the activity of the promoter of the present invention. Additionally, microbial culture supernatants, natural components from plants or marine organisms, or extracts from animal tissues may also be used as the test compound for screening. Further, compounds (including peptides) prepared by chemical or biological modification of the compounds (including peptides) selected by the screening method of the present invention may also be used.

The analysis method of the present invention includes a process of analyzing a test compound about whether or not the test compound inhibits the activity of the gene regulation sequences of the enzymes of the present invention, including (i) a step of putting a cell transfected with an expression vector comprising the gene regulation sequences of the enzymes of the present invention into contact with the test compound, and (ii) a step of detecting the activity of said gene regulation sequences.

These gene regulation sequences may be the promoter.

An inhibitor of the fungal siderophore biosynthesis directed against the 5′ non-translated region of the genes characterizes herein above mainly lead to a repression of gene expression. Accordingly, said repression may be achieved by suppressing expression of the gene, e.g., by specifically suppressing transcription from the respective promoter by suitable compounds (inhibitors) or by rendering the promoter less efficient or non-functional by employing said inhibitors.

In another embodiment, cells are transfected with nucleic acid constructs encoding a reporter gene regulated by the gene regulation sequence/promoter of any of the enzymes characterized herein above and comprised in the fungal siderophore biosynthesis, an increase or decrease in the expression of the reporter gene in response to biological or pharmaceutical agents can be analyzed using methods that detect levels or status of protein or mRNA present in the corresponding cell or detect biological activities of the reporter gene. Suitable reporter molecules or labels, which may be used, include radionucleotides, enzymes, fluorescent, chemiluminescent or chromogenic agents as well as substrates, co-factors, inhibitors, magnetic particles, and the like. Designing such drug screening assays are well known in the art; see Harvey ed., ‘Advances in drug discovery techniques’, John Wiley and Sons, 1998; Vogel and Vogel eds., ‘Drug discovery and evaluation: Pharmaceutical assays’, Springer-Verlag Berlin, 1997). The screening assays provided herein for inhibitors of the fungal siderophore biosynthesis may, accordingly, also comprise in vitro tests using animal cells. An in vitro model can be used for screening libraries of compounds in any of a variety of drug screening techniques provided herein.

When using the term “to deplete” in the context of the present invention, it means that due to a process of sequence-specific, post-transcriptional gene silencing (PTGS) expression of a desired gene, for example, L-ornithine N⁵-oxygenase gene expression, N⁵-transacylase gene expression, non-ribosomal peptide synthetase gene expression, enoyl CoA hydratase gene expression or N²-transacetylase gene expression, is suppressed. Accordingly, the RNA encoding, for example L-ornithine N⁵-oxygenase, N⁵-transacylase, non-ribosomal peptide synthetase, enoyl CoA hydratase and N²-transacetylase and/or fragments thereof may be partially or completely degraded by the mechanism/pathway of RNAi and, thus, may not be translated or only translated in insufficient amounts which causes a phenotype almost resembling or resembling that of a knock-out of the respective gene. Consequently, for example, no or at least to less of an enzyme selected from the group consisting of L-ornithine N⁵-oxygenase, N⁵-transacylase, non-ribosomal peptide synthetase, enoyl CoA hydratase and N²-transacetylase will be produced.

20- to 50-nucleotide RNAs, preferably 15, 18, 20, 21, 25, 30, 35, 40, 45 and 50-nucleotide RNAs are chemically synthesized using appropriately protected ribonucleoside phosphoramidites and a conventional DNA/RNA synthesizer. Most conveniently, siRNAs and the like are obtained from commercial RNA oligo synthesis suppliers, which sell RNA-synthesis products of different quality and costs. In general, 20 to 50-nucleotide RNAs are not too difficult to synthesize and are readily provided in a quality suitable for RNAi. However, specific gene silencing may also be obtained by longer RNA, for example long dsRNA which may comprise even 500 nt; see, inter alia, Paddison (2002), PNAS 99, 1443-1448. The preferred targeted region is selected from a given nucleic acid sequence beginning, inter alia, 50 to 100 nt downstream of the start codon.

Dosage, pharmaceutical preparation and delivery of inhibitors of fungal siderophore biosynthesis as described herein for use in accordance with the present invention may be formulated in conventional manner according to methods found in the art, using one or more physiological carriers or excipients, see, for example Ansel et al., “Pharmaceutical Dosage Forms and Drug Delivery Systems”, 7^{th} edition, Lippincott Williams & Wilkins Publishers, 1999. Thus, the fungal siderophore biosynthesis inhibitors and its physiologically acceptable salts and solvates may be formulated for administration by inhalation, insufflation (either through the mouth, or nose), oral, buccal, parenteral, or rectal administration.

The pharmaceutical composition may be administered with a physiologically acceptable carrier to a patient, as described herein. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the inhibitor described herein, preferably in purified form, together with a suitable amount of carrier so as to provide the form for proper administration to the patient. The formulation should suit the mode of administration.

In another embodiment, the composition is formulated in accordance with routine procedures as a pharmaceutical composition adapted for intravenous administration to human beings. Typically, compositions for intravenous administration are solutions in sterile isotonic aqueous buffer. In a preferred embodiment, the pharmaceutical compositions are in a water-soluble form, such as pharmaceutically acceptable salts, which is meant to include both acid and base addition salts. The administration of the candidate agents of the present invention can be done in a variety of ways as discussed above, including, but not limited to, orally, subcutaneously, intravenously, intranasally, transdermally, intranodally, peritumourally, intratumourally, intrarectally, intraperitoneally, intramuscularly, intrapulmonary, vaginally, rectally, or intraocularly. Where necessary, the composition may also include a solubilizing agent and a local anesthetic such as lignocaine to ease pain at the site of the injection. Generally, the ingredients are supplied either separately or mixed together in unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

It is preferred that for oral administration, the pharmaceutical composition of the fungal siderophore biosynthesis inhibitors may take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration may take the form of, for example, solutions, syrups, or suspensions, or may be presented as a dry product for constitution with water or other suitable vehicle before use. Such liquid preparation may be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, soric acids). The preparations may also contain buffer salts, flavouring, coloring and sweetening agents as deemed appropriate. Preparations for oral administration may be suitably formulated to give controlled release of the fungal siderophore biosynthesis inhibitors.

Preferably, for administration by inhalation, the fungal siderophore biosynthesis inhibitors for use according to the present invention is conveniently delivered in the form of an aerosol spray presentation from a pressurised pack or a nebulizer, with the use of a suitable propellant (e.g., dichlorodifluoromethane, trichlorofluoromethane, dichlorotetrafluoroethane, carbon dioxide or other suitable gas). In the case of a pressurised aerosol, the dosage unit may be determined by providing a valve to deliver a metered amount. Capsules and cartridges of, for example, gelatine, for use in an inhaler or insufflator may be formulated containing a powder mix of the fungal siderophore biosynthesis inhibitors and a suitable powder base such as lactose or starch.

It is also preferred that a fungal siderophore biosynthesis inhibitor may be formulated for parenteral administration by injection, for example, by bolus injection or continuous infusion. Site of injections include intravenous, intraperitoneal or sub-cutaneous. Formulations for injection may be presented in units dosage form (e.g., in phial, in multi-dose container), and with an added preservative. The fungal siderophore biosynthesis inhibitors may take such forms as suspensions, solutions or emulsions in oily or aqueous vehicles, and may contain formulatory agents such as suspending, stabilizing, or dispersing agents. Alternatively, the agent may be in powder form for constitution with a suitable vehicle (e.g., sterile pyrogen-free water) before use.

Fungal siderophore biosynthesis inhibitors may, if desired, be presented in a pack, or dispenser device which may contain one or more unit dosage forms containing the said agent. The pack may for example comprise metal or plastic foil, such as blister pack. The pack or dispenser device may be accompanied with instruction for administration.

In another embodiment of the present invention the inhibitor of fungal siderophore biosynthesis is administered in combination with one or more agents known in the art to be effective against fungi, in particular effective against Aspergillus species and particularly effective against Aspergillus fumigatus. Examples of agents which are effective against Aspergillus species are amphotericin B, itraconazole, voriconazole, echinocandins, posaconazole, ravuconazole, glucan synthesis inhibitors (e.g., caspofungin, V-echinocandin, FK463) or liposomal nystatin. In particular, voriconazole is preferred for being effective against Aspergillus fumigatus. Also preferred agents for being effective against Aspergillus fumigatus are the aforementioned glucan synthesis inhibitors or liposomal nystatin. The term “agents which are effective against” means agents that impair or inhibit growth of an Aspergillus species, in particular Aspergillus fumigatus or agents that kill an Aspergillus species, in particular Aspergillus fumigatus or attenuate virulence of the aforementioned fungi.

In addition, the present invention envisages the use of an inhibitor of siderophore biosynthesis in Aspergillus species, particularly in Aspergillus fumigatus for the preparation of a pharmaceutical composition for the prevention and/or treatment of a disease associated with infection of an Aspergillus species described hereinbelow, in particular with Aspergillus fumigatus. In a preferred embodiment said disease is aspergillose or coccidiosis.

Aspergillus species are well-known to play a role in three different clinical settings in man: (i) opportunistic infections; (ii) allergic states; and (iii) toxicoses. Immunosuppression is the major factor predisposing to development of opportunistic infections (Ho, Crit. Rev Oncol Hematol 34, (2000), 55-69. These infections may present in a wide spectrum, varying from local involvement to dissemination and as a whole called aspergillosis. Among all filamentous fungi, Aspergillus is in general the most commonly isolated one in invasive infections. It is the second most commonly recovered fungus in opportunistic mycoses following Candida. Almost any organ or system in the human body may be involved. Onychomycosis, sinusitis, cerebral aspergillosis, meningitis, endocarditis, myocarditis, pulmonary aspergillosis, osteomyelitis, otomycosis, endophthalmitis, cutaneous aspergillosis, hepatosplenic aspergillosis, as well as Aspergillus fungemia, and disseminated aspergillosis may develop. Nosocomial occurrence of aspergillosis due to catheters and other devices is also likely. Construction in hospital environments constitutes a major risk for development of aspergillosis particularly in neutropenic patients.

Aspergillus spp. may also be local colonizers in previously developed lung cavities due to tuberculosis, sarcoidosis, bronchiectasis, pneumoconiosis, ankylosing spondylitis or neoplasms, presenting as a distinct clinical entity, called aspergilloma. Aspergilloma may also occur in kidneys.

Some Aspergillus antigens are fungal allergens and may initiate allergic bronchopulmonary aspergillosis particularly in atopic host. Some Aspergillus spp. produce various mycotoxins. These mycotoxins, by chronic ingestion, have proven to possess carcinogenic potential particularly in animals. Among these mycotoxins, aflatoxin is well-known and may induce hepatocellular carcinoma. It is mostly produced by Aspergillus flavus and contaminates foodstuff, such as peanuts. Aspergillus spp. can cause infections in animals as well as in man. In birds, respiratory infections may develop due to Aspergillus. It may induce mycotic abortion in the cattle and the sheep. Ingestion of high amounts of aflatoxin may induce lethal effects in poultry animals fed with grain contaminated with the toxin. Accordingly, it is envisaged that the aforementioned diseases can be treated and/or prevented with an inhibitor described herein or with an inhibitor identified by the methods for screening as described herein. It is also envisaged that the pharmaceutical compositions of the present invention and the medical uses and methods provided herein are employed in disorders when fungal infections occur an additional disorder, for example in immuno-suppressed patients. These patients may, inter alia, suffer from chronic granulomatous disease, bone marrow transplantation, acute leukaemia, cancer (as well as cytotoxic treatment) or HIV infection (AIDS).

In another aspect the present invention relates to a method of treating and/or preventing diseases associated with fungal infections comprising administering a therapeutically effective amount of a pharmaceutical composition an inhibitor of fungal siderophore biosynthesis to a subject suffering from said disorder.

In the context of the present invention the term “subject” means an individual in need of a treatment of an affective disorder. Preferably, the subject is a vertebrate, even more preferred a mammal, particularly preferred a human.

The term “administered” means administration of a therapeutically effective dose of the aforementioned inhibitor to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents maybe administered alone or in combination with other treatments.

The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the candidate agents may be directly applied as a solution dry spray.

The attending physician and clinical factors will determine the dosage regimen. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors.

The dosages are preferably given once a week, however, during progression of the treatment the dosages can be given in much longer time intervals and in need can be given in much shorter time intervals, e.g., daily. In a preferred case the immune response is monitored using herein described methods and further methods known to those skilled in the art and dosages are optimized, e.g., in time, amount and/or composition. Dosages will vary but a preferred dosage for intravenous administration of DNA encoding a potential inhibitor of fungal siderophore biosynthesis as described herein is from approximately 10⁶ to 10¹² copies of the DNA molecule. If the regimen is a continuous infusion, it should also be in the range of 1 μg to 10 mg units per kilogram of body weight per minute, respectively. Progress can be monitored by periodic assessment. The pharmaceutical composition of the invention may be administered locally or systemically. Administration will preferably be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium ion solution, Ringer's dextrose, dextrose and sodium ion, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

It is also envisaged that the pharmaceutical compositions are employed in co-therapy approaches, i.e. in co-administration with other medicaments or drugs, for example other drugs for preventing, treating or ameliorating the diseases or disorders associated with fungal infection, in particular with infection of Aspergillus species, more particular with infection of Aspergillus fumigatus as described herein.

Another aspect of the present invention is a diagnostic composition comprising the nucleic acid molecules described herein or the antibodies which preferably specifically bind to the polypeptides involved in siderophore biosynthesis described herein. A preferred aspect is diagnosing fungal infection, in particular, infection by Aspergillus spec., more particularly, by A. fumigatus by PCR techniques or immuno assays techniques known in the art.

Even today, invasive aspergillosis remains difficult to diagnose, in particular when it is in the early stages (Latge (Clin. Microbiol. Rev. 12 (1999), 310-50)). In fact, histopathological evidence of mycelial growth in tissue has to be provided to prove aspergillosis. Unfortunately, this is most often demonstrated only at autopsy. Features currently considered in the diagnosis include (i) a positive CT scan, (ii) culture and/or microscopic evidence of disease, and (iii) detection of Aspergillus antigen(s) in serum, and (iv) PCR-mediated detection of genomic DNA of A. fumigatus. Clinical symptoms are usually too nonspecific to be helpful in narrowing the focus to aspergillosis. The use of culture and microscopic examination of respiratory tract specimens has been criticized because of the presence of airborne conidia of Aspergillus and the possibility that a positive culture from such specimens results from accidental contamination. Similarly, PCR-mediated detection is prone to lead to false-positive results. A prerequisite for antigen-mediated diagnosis of aspergillosis is that the antigen is released from the fungus because the fungus itself is not disseminating constantly into the blood stream during infection and blood samples are the most commonly used diagnostic sample. Currently, detection of the circulating antigen galactomannan (a component of the cell wall which is also released by the fungus) is the method most used. However, a high percentage of false-negative results as well as false-positive results were recorded. Consequently it is clear that despite significant progress in the serological diagnosis of aspergillosis by antigen detection, the sensitivity and specificity of detection must be improved. However, it is shown in various Examples herein that genes required for siderophore biosynthesis and in particular for production of triacetylfusarinine C are essential for virulence of A. fumigatus. Consequently, siderophores, and in particular traicetylfusarinine C, are produced in vivo during pathogenic growth. During iron depleted conditions in vitro, A. fumigatus excretes high amounts of siderophores into the medium and pathogenic growth apparently resembles growth during iron starvation. Therefore, high siderophore excretion is expected during virulent-growth. Consequently, detection of siderophores, and in particular of triacetylfusarinine, can be used as a diagnostic marker of invasive aspergillosis. Siderophores as triacetylfusarinine C can be detected with high specificity and sensitivity by either serological methods using specific antibodies or by mass spectrometry.

Since animals, for example, mammals, preferably humans, do not have genes encoding polypeptides involved in siderophore biosynthesis, the present invention relates in another aspect to a diagnostic composition comprising the nucleic acid molecule(s), the vector, the host, the polypeptide or the antibody described herein, optionally further comprising suitable means for detection. Thus, it is envisaged that, for example, the nucleic acid molecules or the antibodies described herein can be used for detecting fungal infections, in particular, infections with one or more Aspergillus species, preferably, Aspergillus fumigatus. Of course, and as already described herein above, also the siderophores fusarinine C and/or triacetylfusarinine C can be detected using, for example, specific antibodies directed against these molecules, HPLC or mass spectrometry.

Moreover, the present invention also relates to a kit comprising the nucleic acid molecule(s), the vector, the host, the polypeptide or the antibody described herein or antibodies specifically binding fusarinine C and/or triacetylfusarinine C. In particular, the nucleic acid molecules (or fragments thereof) as provided herein are useful in diagnostic methods, comprising, inter alia, the PCR-technology.

Advantageously, the kit of the present invention further comprises, optionally (a) reaction buffer(s), storage solutions and/or remaining reagents or materials required for the conduct of scientific or diagnostic assays or the like. Furthermore, parts of the kit of the invention can be packaged individually in vials or bottles or in combination in containers or multicontainer units.

The kit of the present invention may be advantageously used, inter alia, for detecting one or more of the nucleic acid molecules described herein which encode (a) polypeptide(s) involved in fungal siderophore biosynthesis as described herein. Said kit may also be used to detect one or more of the polypeptides involved in siderophore biosynthesis as described herein. Thus, said kit could be, for example, employed in a variety of applications, e.g., as diagnostic kit, as research tool or therapeutic tool. Additionally, the kit of the invention may contain means for detection suitable for scientific, medical and/or diagnostic purposes. The manufacture of the kits follows preferably standard procedures which are known to the person skilled in the art.

In addition, the nucleic acid molecules, the polypeptide, the vector, the host cell or the antibody of the present invention or the antibodies specific, for fusarinine C and/or triacetylfusarinine C are used for the preparation of a diagnostic composition for detecting (a) fungal infection(s), preferably, an Aspergillus species infection, more preferably, infection with Aspergillus fumigatus in a sample.

Moreover, the nucleic acid molecules, the polypeptide, the vector, the host cell or the antibody of the present invention or the antibodies specific for fusarinine C and/or triacetylfusarinine C are used for the preparation of a diagnostic composition for the detection of, e.g., aspergillosis or coccidiosis in a sample.

In accordance with the present invention by the term “sample” is intended any biological sample obtained from an individual, cell line, tissue culture, or other source containing polynucleotides or polypeptides or portions thereof. As indicated, biological samples include body fluids (such as blood, sera, plasma, urine, synovial fluid and spinal fluid) and tissue sources found to express the polynucleotides of the present invention. Methods for obtaining tissue biopsies and body fluids from mammals are well known in the art. A biological sample which includes genomic DNA, mRNA or proteins is preferred as a source.

The diagnostic composition optionally comprises suitable means for detection. The nucleic acid molecule(s), vector(s), host(s), antibody(ies), and polypeptide(s) described above are, for example, suitable for use in immunoassays in which they can be utilized in liquid phase or bound to a solid phase carrier. Examples of well-known carriers include glass, polystyrene, polyvinyl ion, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

Solid phase carriers are known to those in the art and may comprise polystyrene beads, latex beads, magnetic beads, colloid metal particles, glass and/or silicon chips and surfaces, nitrocellulose strips, membranes, sheets, duracytes and the walls of wells of a reaction tray, plastic tubes or other test tubes. Suitable methods of immobilizing nucleic acid molecule(s), vector(s), host(s), antibody(ies), aptamer(s), polypeptide(s), etc. on solid phases include but are not limited to ionic, hydrophobic, covalent interactions or (chemical) crosslinking and the like. Examples of immunoassays which can utilize said compounds of the invention are competitive and non-competitive immunoassays in either a direct or indirect format. Commonly used detection assays can comprise radioisotopic or non-radioisotopic methods. Examples of such immunoassays are the radioimmunoassay (RIA), the sandwich (immunometric assay) and the Northern or Southern blot assay. Furthermore, these detection methods comprise, inter alia, IRMA (Immune Radioimmunometric Assay), EIA (Enzyme Immuno Assay), ELISA (Enzyme Linked Immuno Assay), FIA (Fluorescent Immuno Assay), and CLIA (Chemiluminescent Immune Assay). Furthermore, the diagnostic compounds of the present invention may be are employed in techniques like FRET (Fluorescence Resonance Energy Transfer) assays.

Appropriate labels and methods for labeling are known to those of ordinary skill in the art. Examples of the types of labels which can be used in the present invention include inter alia, fluorochromes (like fluorescein, rhodamine, Texas Red, etc.), enzymes (like horse radish peroxidase, β-galactosidase, alkaline phosphatase), radioactive isotopes (like ³²P, ³³P, ³⁵S or ¹²⁵I), biotin, digoxygenin, colloidal metals, chemi- or bioluminescent compounds (like dioxetanes, luminol or acridiniums).

A variety of techniques are available for labeling biomolecules, are well known to the person skilled in the art and are considered to be within the scope of the present invention and comprise, inter alia, covalent coupling of enzymes or biotinyl groups, phosphorylations, biotinylations, random priming, nick-translations, tailing (using terminal transferases). Such techniques are, e.g., described in Tijssen, “Practice and theory of enzyme immunoassays”, Burden and von Knippenburg (Eds), Volume 15 (1985); “Basic methods in molecular biology”, Davis L G, Dibmer M D, Battey Elsevier (1990); Mayer, (Eds) “Immunochemical methods in cell and molecular biology” Academic Press, London (1987); or in the series “Methods in Enzymology”, Academic Press, Inc. Detection methods comprise, but are not limited to, autoradiography, fluorescence microscopy, direct and indirect enzymatic reactions, etc.

A diagnostic application in which the kit or the diagnostic composition of the present invention is used comprises any amplification technique. The term “amplification technique” refers to any method that allows the generation of a multitude of identical or essentially identical (i.e. at least 95% more preferred at least 98%, even more preferred at least 99% and most preferred at least 99.5% such as 99.9% identical) nucleic acid molecules or parts thereof. Such methods are well established in the art; see Sambrook et al. “Molecular Cloning, A Laboratory Manual”, 2^{nd} edition 1989, CSH Press, Cold Spring Harbor. Various PCR techniques, including real-time PCR are reviewed, for example, by Ding, J. Biochem. Mol. Biol. 37 (2004), 1-10.

PCR is an example of an amplification technique. PCR is a powerful technique used to amplify DNA millions of fold, by repeated replication of a template, in a short period of time. The process utilizes sets of specific in vitro synthesized oligonucleotides to prime DNA synthesis. The design of the primers is dependent upon the sequences of the DNA that is desired to be analyzed. It is known that the length of a primer results from different parameters (Gillam (1979), Gene 8, 81-97; Innis (1990), PCR Protocols: A guide to methods and applications, Academic Press, San Diego, USA). Preferably, the primer should only hybridize or bind to a specific region of a target nucleotide sequence. The length of a primer that statistically hybridizes only to one region of a target nucleotide sequence can be calculated by the following formula: (¼)^{x} (whereby x is the length of the primer). For example a hepta- or octanucleotide would be sufficient to bind statistically only once on a sequence of 37 kb. However, it is known that a primer exactly matching to a complementary template strand must be at least 9 base pairs in length, otherwise no stable-double strand can be generated (Goulian (1973), Biochemistry 12, 2893-2901). It is also envisaged that computer-based algorithms can be used to design primers capable of amplifying the nucleic acid molecules of the invention. Preferably, the primers of the invention are at least 10 nucleotides in length, more preferred at least 12 nucleotides in length, even more preferred at least 15 nucleotides in length, particularly preferred at least 18 nucleotides in length, even more particularly preferred at least 20 nucleotides in length and most preferably at least 25 nucleotides in length. The invention, however, can also be carried out with primers which are shorter or longer. The person skilled in the art can readily design primers to be used in the diagnostic method of the invention, particular on basis of the nucleic acid molecules provided herein and homologous molecules as defined herein above. In a diagnostic method also the appended examples provide for means and methods how specific primers (or probes) may be generated. “Primers” and “probes” are particularly useful in the diagnostic methods provided herein.

The PCR technique is carried out through many cycles (usually 20-50) of melting the template at high temperature, allowing the primers to anneal to complimentary sequences within the template and then replicating the template with DNA polymerase. The process has been automated with the use of thermostable DNA polymerases isolated from bacteria that grow in thermal vents in the ocean or hot springs. During the first round of replication a single copy of DNA is converted to two copies and so on resulting in an exponential increase in the number of copies of the sequences targeted by the primers. After just 20 cycles a single copy of DNA is amplified over 2,000,000 fold.

### THE FIGURES SHOW

FIG. 1: Extracellular (triacetylfusarinine C, TAFC) and intracellular (ferricrocin, FC) siderophore production of A. fumigatus. A. Wild type strains after growth for 24 h in Aspergillus minimal medium (AMM) (Pontecorvo (1953), Adv. Genet. 5, 141-124) during iron starvation (−Fe) and iron-replete conditions (10 μM FeSO₄, +Fe). B. Wild type and mutant strains after growth at 37° C. for 12 h and 24 h during iron starvation. The siderophore production was analyzed by reversed-phase HPLC analysis as described in Oberegger (2001), loc. cit. and normalized to that of CEA10 during iron starvation for 24 h.

FIG. 2: Iron-regulated expression of sidA, ftrA, and fetC in A. fumigatus CEA10. Fungal strains were grown at 37° C. for 24 h in AMM containing 10 μM FeSO₄ (+Fe) or lacking iron (−Fe) iron. Total RNA was isolated from the harvested mycelia and subject to Northern analysis (Sambrook, Russell (2001), loc. cit.). As a control for loading and RNA quality, blots were hybridized with the β-tubuline encoding tubA gene. The lanes on the Northern blot shown are as follows: left lane, iron replete condition (10 μM FeSO₄).; right lane, iron depleted conditions.

FIG. 3: A. Growth phenotypes of wild type and mutant strains. A. fumigatus possesses two high-affinity iron uptake mechanisms. 10⁴ conidia of the respective strain were point-inoculated on AMM plates containing the respective iron source, and incubated for 48 h at 37° C. Blood agar was AMM containing 5% sheep blood.

FIG. 4: A sidA deficient strain was in contrast to a ftrA-deficient strain and a reconstituted sidA strain avirulent (The parental wild type A. fumigatus strain ATCC46645 showed the same virulence as ΔftrA and sidA^{r}, data not shown). Fifteen mice per group were infected by intranasal instillation of 2×10⁵ conidiospores.

FIG. 5: Southern blot analysis of ΔsidA and sidA^{R} The lanes on the Southern blot shown are as follows: left lane, sidA^{R} (reconstituted ΔsidA); middle lane, wild type ATCC46645, right lane, ΔsidA.

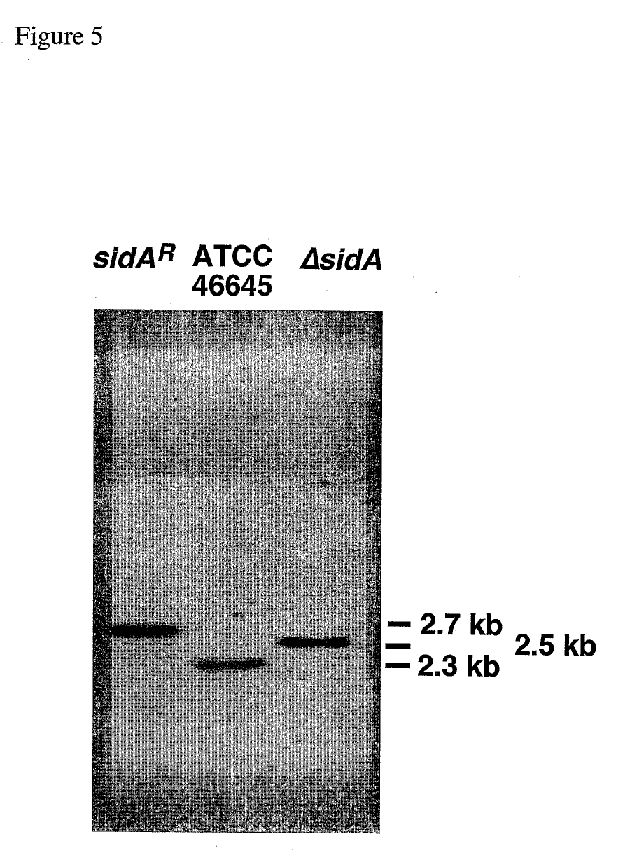

FIG. 6: Southern blot analysis of ΔftrA The lanes on the Southern blot shown are as follows: left lane, ΔftrA; right lane, wild type ATCC46645.

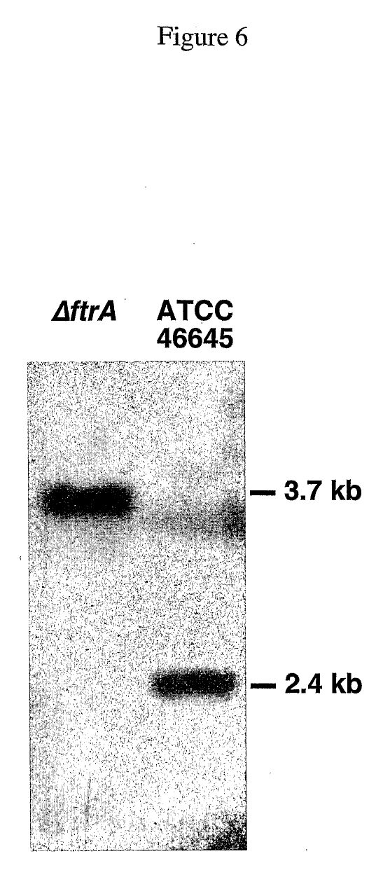

FIG. 7: Growth inhibition of siderophore negative A fumigatus (corresponds to wild type plus specific inhibitor of siderophore biosynthesis) by bathophenanthroline disulfonic acid (BPS) and blood—antagonist action of ferricrocin. wt, A. fumigatus wild type strain ATCC46645; ΔAf-sidA, OMO-deficient A. fumigatus strain

FIG. 8: Siderophore biosynthesis in fungi The Figure shows a schematic overview about the proposed biosynthesis pathway for siderophore biosynthesis in fungi.

FIG. 9: Iron-regulated expression of at1, sidD and at2 in A. fumigatus ATCC46645 Northern analysis was performed as described in FIG. 2. The lanes on the Northern blot shown are as follows: left lane, iron-replete conditions (10 μM FeSO₄); right lane, iron depleted conditions.

FIG. 10: Southern blot analysis of at1, sidD and at2

FIG. 11: Extracellular (triacetylfusarinine C, TAFC; fusarinine C, FSC) and intracellular (ferricrocin, FC) siderophore biosynthesis of A. fumigatus Δat1, ΔsidD and Δat2 during iron depleted conditions. The siderophore production was analyzed as described in FIG. 1. TAFC and FSC production was normalized to TAFC production of the wild type ATCC46645; FC production was normalized to that of FC production of ATCC46645.

FIG. 12: Growth phenotypes of wild type and Δat1, ΔsidD and Δat2 mutant strains. Growth assays were performed during iron replete conditions (+Fe, 10 μM FeSO₄), iron depleted conditions (−Fe), iron depleted conditions in the presence of bathophenantroline-disulfonate (BPS, 200 μM) and on blood agar (blood) as described in FIG. 3, radial growth was scored at 48 h.

FIG. 13: Δat1, ΔsidD and Δat2 display reduced capacity to establish systemic infection in a murine model. The virulence assay was performed as described in FIG. 5 but with 5 mice per group.

The invention will now be described by reference to the following biological examples which are merely illustrative and are not construed as a limitation of the scope of the present invention.

### EXAMPLE 1

### cDNA-Sequence and Northern Analysis of Aspergillus fumigatus

RNA was isolated using TRI Reagent™ (Sigma). The cDNA sequences of Af-sidA and Af-sreA were analyzed by reverse transcribed-PCR using Superscript™ (Invitrogen). The 5′- and 3′-ends were determined with the GenRacer™ method (Invitrogen) using total RNA.

For Northern analysis, 5 μg of total RNA was electrophoresed on 1.2% agarose-2.2 M formaldehyde gels and blotted onto Hybond N membranes (Amersham). The hybridization probes used in this study were generated by PCR using oligonucleotides 5′-AACTACCTCCACCAGAAG and 5′-GAACGGCAATGTTGTAAG for sidA, 5′-GGGACAAGAGCAAGATGC and 5′-CCCAGTAGAGGATGCAAG for ftra, 5′-GTGACCGATCCCAAGAAC and 5′-GGATGGGAATGTCTTGTG for fetC, and 5′-ATATGTTCCTCGTGCCGTTC and 5′-CCTTACCACGGAAAATGGCA for β-tubulin encoding tubA.

### EXAMPLE 2

### Siderophore Production in Aspergillus fumigatus

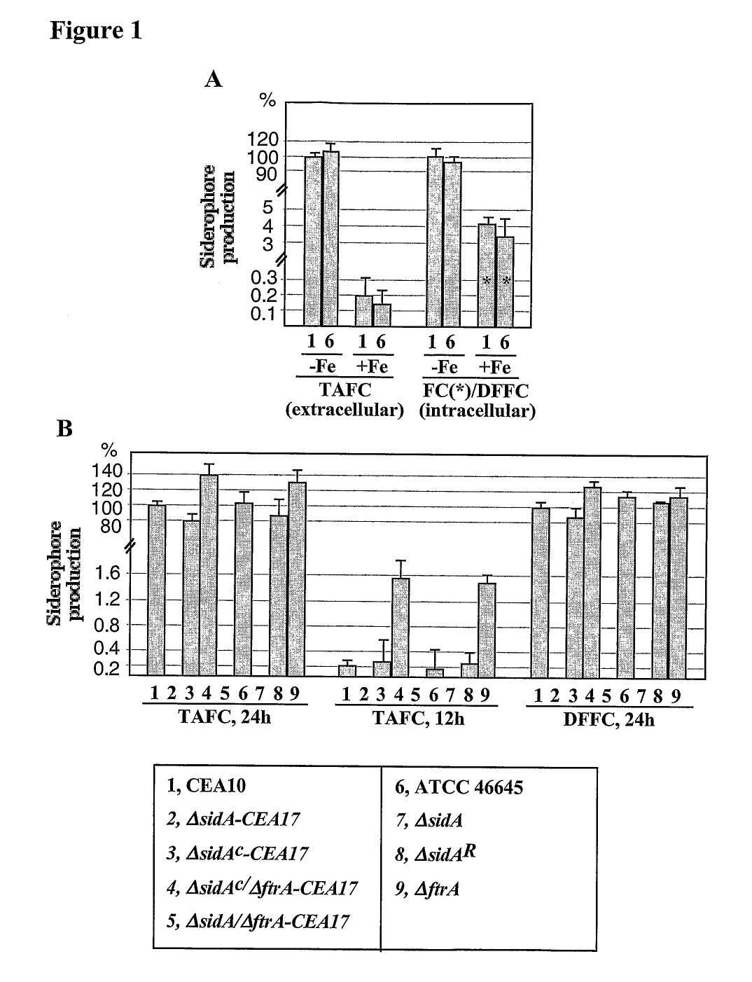

In a first step to study the role of the siderophore system in iron homeostasis and its impact on virulence of A. fumigatus, siderophore production was analyzed (FIG. 1). During iron starvation, A. fumigatus ATCC46645 and CEA10 (the genotypes of the strains used in this study are summarized in Table 1, infra, excreted triacetylfusarinine C and accumulated intracellular desferriferricrocin (iron-free ferricrocin)). During iron-replete conditions the mycelia contained low amounts of the siderophore ferricrocin and excreted very low amounts of extracellular siderophores.

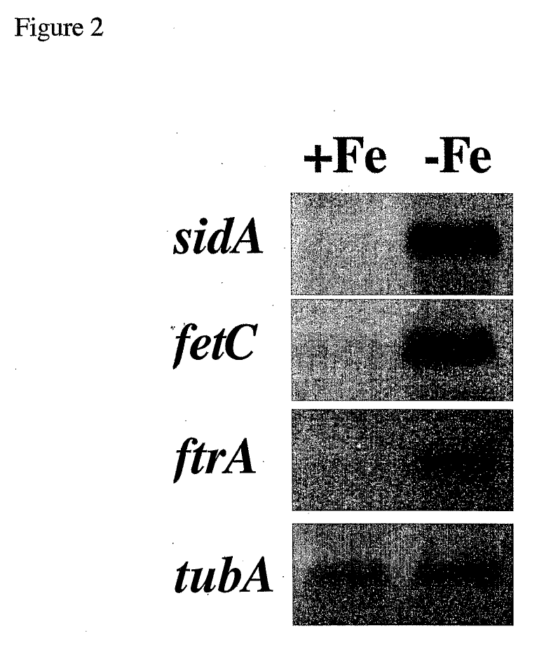

Therefore, the A. fumigatus siderophore system resembles that of A. nidulans (Oberegger, Mol. Microbiol. 41 (2001), 1077-1089). A search in the genome sequence of A. fumigatus revealed one putative L-ornithine-N⁵-monooxygenase encoding gene, termed sidA. Comparison of the genomic and cDNA sequences revealed the presence of one intron in sidA. The deduced amino acid sequence of SidA is 501 amino acids in length, contains all signatures typical for hydroxylases involved in siderophore biosynthesis and displays 78% identity with A. nidulans SidA. Northern analysis indicated that Af-sidA expression is upregulated by iron starvation (FIG. 2).

TABLE 1
A. fumigatus strains used:

| STRAIN | genotype | reference |
|---|---|---|
| CEA10 | wild type | d′Enfert (1996), Infect. |
| | | Immun. 64, 4401-4405 |
| CEA17 | pyrG⁻ | d′Enfert (1996), loc. cit. |
| ΔsidACEA17 | CEA17, ΔsidA::pyrG | described herein |
| sidA^{c}CEA17 | ΔsidACEA17, (p)::sidA | described herein |
| ΔsidA/Δ | ΔsidACEA17, ΔftrA::hph | described herein |
| ftrACEA17 | | |
| sidA^{c}/ΔftrACEA17 | ΔsidA/Δ ftrACEA17, | described herein |
| | (p)::sidA | |
| ΔsidA/ftrA^{c}CEA17 | ΔsidA/Δ ftrACEA17, | described herein |
| | (p)::ftrA | |
| ATCC46645 | wild type | American Type Culture |
| | | Collection |
| ΔsidA | ATCC46645, ΔsidA::hph | described herein |
| ΔftrA | ATCC46645, ΔftrA::hph | described herein |
| sidA^{r} | ΔsidA, ΔsidA::sidA | described herein |
| ΔsidD | ATCC46645, ΔsidD::hph | described herein |
| Δat1 | ATCC46645, Δat1::hph | described herein |
| Δat2 | ATCC46645, | described herein |
| | Δat2::hphTK | |

### EXAMPLE 3

### Disruption of sidA

For generation of the Af-sidA disruption vectors, a 5.1-kb fragment containing Af-sidA was amplified by PCR using primers 5′-TCACCTGCTCGTCATGCGTC and 5′-GGAGTATCTAGATGCGACACTACTCTC, subcloned into the pGEM-T vector (Promega). The resulting plasmid was sequenced and termed pSIDA. For generation of ΔsidA-CEA17, an internal 1.5-kb SmaI-ClaI fragment was replaced by a 1.9-kb SmaI-ClaI fragment of vector pAfpyrG containing the pyrG selection marker (Weidner, Curr. Genet. 33 (1998), 378-385). For transformation of CEA17, the gel-purified 5.5-kb XbaI fragment was used. In the generated mutant allele of ΔsidA-CEA17 the deleted region encompasses the region encoding amino acids 174-501 and 476 bp of the 3′-downstream region of sidA.

For generation of ΔsidA, the internal 2-kb BglII-HindIII fragment of pSIDA was replaced by the 4.0-kb BglII-HindIII fragment of vector pAN7-1 (Punt, Gene 56 (1987), 117-124) containing the hygromycin B (hph) selection marker. For transformation of A. fumigatus ATCC46645, the gel-purified 6.9-kb BssHII fragment was used. In the generated mutant allele of ΔsidA, the deleted region encompasses the entire coding region, 279 bp of the 3′-downstream and 137 bp of the 5′-upstream region of sidA.

### EXAMPLE 4

### Complementation of sidA Deficiency

For complementation of sidA-deficiency of strains ΔsidACEA17 and ΔsidA/ΔftrA-CEA17 a single copy of pSIDA was ectopically integrated by transformation, which gave strains sidA^{c}CEA17 and sidA^{c}/ΔftrA-CEA17, respectively.

For reconstitution of sidA in the ΔsidA strain, a Bpu1102I restriction site in the 3′-noncoding region of sidA in pSIDA was deleted by digestion and blunt-ending, which generated a Mwol restriction site. For transformation of ΔsidA, the gel-purified 4.9-kb BssHII fragment was used. This procedure allowed to distinguish the homologously reconstituted sidA^{c} strain from the ATCC46645.

### EXAMPLE 5

### Southern Blot Analysis of ΔsidA and sidA^{R}

For Southern analysis genomic DNA was digested with NcoI/Bpu11021, subject to electrophoresis, blotted onto nylon membrane and hybridized with a probe amplified with 5′-CACCGCTTGAAACCCAGAAT and 5′-GGAGTATCTAGATGCGACACTACTCTC by techniques known in the art. Consistent with the genotypes, the probe detected fragments in the length of 2.7, 2.3, and 2.5 kb in sidA^{R}, ATCC46645, and ΔsidA, respectively.

### EXAMPLE 6

### Disruption of ftrA

To construct the ΔftrA alleles, a 5.0-kb fragment was amplified using primers 5′-GTGGGATTGCTGATGCTG and 5′-AAGATTGATATCAACACCTTTCCCATAAC. The amplification product was subcloned into the pGEM-T, the plasmid termed pFTRA, and the insert sequence-confirmed. Subsequently, an internal 1.7-kb NheI-HindIII fragment was replaced by the 3.2-kb NheI-HindIII fragment of vector pAN7-1 carrying the hph selection marker. For transformation of A. fumigatus ΔsidACEA17 and ATCC46645, the gel-purified 6.5-kb EcoRV fragment was used. The deleted region encompasses the region encoding amino acids 82-370 and 764 bp of the 3′-downstream region of ftrA.

### EXAMPLE 7

### Complementation of ftrA Deficiency

For complementation of ftrA-deficiency of ΔsidA/ΔftrACEA17 one copy of pFTRA was ectopically integrated by transformation, yielding strain ΔsidA/ftrA^{c}-CEA17.

### EXAMPLE 8

### Southern Blot Analysis of ΔftrA

For Southern analysis genomic DNA was digested with EcoRV, subject to electrophoresis, blotted onto nylon membrane and hybridized with a probe amplified with 5′-AAGATTGATATCAACACCTTTCCCATAAC and 5′-GTGGCCTGCCTTCCCTCC using techniques well known in the art. Consistent with the genotypes, the probe detected a 2.4 kb fragment in ATCC46645 and a 3.7 kb fragment in ΔftrA.

### EXAMPLE 9

### Transformation of Aspergillus fumigatus

Transformation of A. fumigatus was carried out as is known in the art for A. nidulans. Selection for pyrG prototrophy was performed as described (Weidner (1998), loc. cit.). Selection for hygromycin B resistance was on plates containing 200 μg hygromycin B (Calbiochem) ml⁻¹. Subsequent to a 24 h-incubation, the plates were overlayed with 5 ml of soft agar containing the same hygromycin concentration. ΔsidA strains containing a reinserted functional Af-sidA copy were screened on -Fe-AMM plates and identified due to their increased growth and sporulation rate. Screening of desired transformants was performed by PCR; single homologous genomic integration was confirmed by Southern blot analysis.

### EXAMPLE 10

### SidA is Involved in Siderophore Production of Aspergillus fumigatus

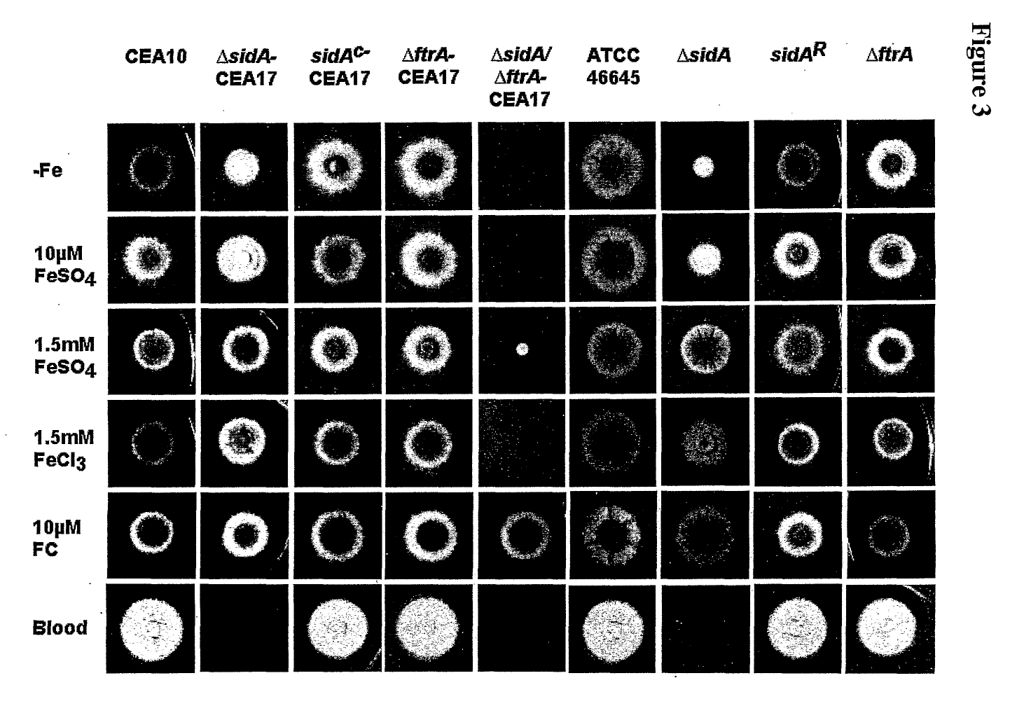

To elucidate the role of sidA a gene deletion mutant from CEA17 by replacement with pyrG was constructed (Weidner (1998), loc. cit.). Reversed-phase-HPLC analysis demonstrated that the sidA-deficient strain ΔsidACEA17 lost the ability to produce both triacetylfusarinine C and ferricrocin (FIG. 1), demonstrating that sidA indeed encodes L-ornithine-N⁵-monooxygenase. During iron depleted and replete conditions ΔsidACEA17 showed a 99% decrease in conidia production, which increased to 35% by supplementation with 1.5 mM iron sulfate or iron chloride and 125% with 10 μM ferricrocin (FIG. 3). These data emphasize the importance of the intracellular siderophore ferricrocin for efficient conidiation in A. fumigatus. In contrast to a siderophore-negative A. nidulans strain, which is not able to grow without siderophore supplementation, SidA-deficiency in A. fumigatus only caused a decrease of the growth rate to 27% during iron starvation, 61% during iron replete conditions, but a lack of growth on blood agar plates (FIG. 3). Complementation of ΔsidACEA17 by ectopic integration of a single wild type copy of sidA, leading to strain sidA^{c}CEA17, cured the defects in conidiation and growth of ΔsidACEA17 demonstrating that the ΔsidACEA17-phenotype is a direct result of loss of sidA. These data indicated that in contrast to A. nidulans, A. fumigatus possesses an additional iron assimilation system. Inspection of the A. fumigatus genome sequence revealed the presence of several putative metalloreductase-encoding genes and, as opposed to A. nidulans, one putative ferroxidase- and one potential high-affinity iron permease-encoding gene, termed fetC and ftrA, which are divergently transcribed from a 1.3 kb intergenic region. Comparison of the genomic and cDNA sequences revealed the presence of five introns in fetC and three introns in ftrA. The deduced amino acid sequence of FetC displays 52% identity with the C. albicans ferroxidase CaFet3 (Eck, Microbiology 145 (1999), 2415-2422), that of FtrA 55% with C. albicans CaFtr1. Northern blot analysis revealed that expression of both genes is upregulated by iron starvation (FIG. 2). Furthermore, ΔsidACEA17 displayed increased sensitivity to the ferrous iron-specific chelator bathophenantroline disulfonate and copper depletion (Askwith, Cell 76 (1994), 403-410), which both functionally inactivate the reductive iron uptake system. Taken together, these data suggested that A. fumigatus has the capacity for reductive iron assimilation.

### EXAMPLE 11

### FtrA is an Essential Component of the Siderophore-Independent Iron Uptake System in Aspergillus fumigatus

To analyze the potential role of FtrA in iron uptake of A. fumigatus we deleted the encoding gene in ΔsidACEA17 by replacement with the hygromycin resistance (hph) marker (Punt (1987), loc. cit.). The resulting double mutant ΔsidA/ΔftrACEA17 was unable to grow unless the growth medium contained ferricrocin (FIG. 3). These data suggest that ftrA indeed encodes an essential component of the siderophore-independent iron uptake system, a fact underscored by the reversion of the ftrA-deletion phenotype by ectopic integration of a single wild-type ftrA copy (FIG. 3, strain ΔsidAftrA^{c}CEA17). The ΔsidA/lΔftrACEA17 double mutant sidA failed to grow on blood agar plates (FIG. 3) or on media containing 10 μM of hemoglobin, hemin, holotransferrin, or ferritin as the sole iron source, which indicates that A. fumigatus lacks specific systems for the uptake of host iron compounds. The slight growth promotion by high amounts of ferrous iron but not ferric iron also suggested the presence of a ferrous uptake system (FIG. 3).

Complementation of sidA-deficiency of ΔsidA/ΔftrACEA17 by ectopic integration of a single wild-type copy of sidA created the ftrA-deficient mutant sidA^{c}/ΔftrACEA17. This strain showed a wild-type growth phenotype (FIG. 3). Compared to CEA10, sidA^{c}/ΔftrACEA17 displayed slightly increased production of triacetylfusarinine C after 24 h but produced about 8-times increased amounts after 12 h of growth during iron depleted conditions. These data demonstrate that lack of FtrA causes an earlier start of siderophore production probably to compensate the lack of reductive iron assimilation.

### EXAMPLE 12

### Preparation of Inocula

A. fumigatus spores for inoculation were propagated on Aspergillus complete medium slants which are known in the art, containing 5 mM ammonium tartrate, 200 mM NaH₂PO₄, and 1.5 mM FeSO₄, at 37° C. for 5 days prior to infection. Conidiospores were harvested on the day of infection using sterile saline (Baxter Healthcare Ltd. England) and filtered through Miracloth (Calbiochem). Following a 5 minute spin at 3000 g spores were washed twice with sterile saline, counted using a haemocytometer and resuspended at a concentration of 5×10⁶ spores/ml.

### EXAMPLE 13

### Virulence Assays

A. fumigatus spores for inoculation were propagated on Aspergillus complete medium slants, containing 5 mM ammonium tartrate, 200 mM NaH₂PO₄, and 1.5 mM FeSO₄, at 37° C. for 5 days prior to infection. Conidiospores were harvested on the day of infection using sterile saline (Baxter Healthcare Ltd. England) and filtered through Miracloth (Calbiochem). Following a 5 minute spin at 3000 g spores were washed twice with sterile saline, counted using a haemocytometer and resuspended at a concentration of 5×10⁶ spores/ml.

All murine infections described in this study were performed under UK Home Office Project Licence PPL/70/5361. Weight-matched (18-22 g) CD1 male mice (Harlan UK Ltd.) were housed in individually vented cages and allowed free access to food and water. Mice were immunosuppressed as previously described (Smith 1994). Briefly, cyclophosphamide (150 mg/kg, Endoxana, Asta Medica) was administered by intraperitoneal injection on days −3, −1, +2, and every subsequent third day throughout all experiments. A single dose of hydrocortisone acetate (112.5 mg/kg, Hydrocortistab, Sovereign Medical) was administered subcutaneously on day −1. All mice received 1 g/l tetracycline hydrochloride (Sigma) and 64 mg/l Ciprofloxacin (Bayer) in drinking water as prophylaxis against bacterial infection. Mice were anaesthetised by halothane inhalation and infected by intranasal instillation of 2×10⁵ conidiospores in 40 μl of saline. Mice were weighed at 24-hourly intervals starting on Day 0. Visual inspections were made twice daily. In the majority of cases the end point for survival experimentation was a 20% reduction in body weight calculated from the day of infection, at which point mice were sacrificed. This usually occurred prior to emergence of more severe indicators of infection such as hunched posture, laboured breathing or moribundity, which served as additional stand-alone end points in cases where weight loss was less than 20%. Survival curves were compared using Kaplan-Meier log rank analysis. Immediately after sacrifice, lungs were removed and fixed in 4% formaldehyde (Sigma). Lungs were embedded in paraffin prior to sectioning and staining with Haemotoxylin and Eosin (H & E) or Grocotts Methiamine Silver (GMS).

### EXAMPLE 14

### SidA is Essential for Virulence in a Murine Model

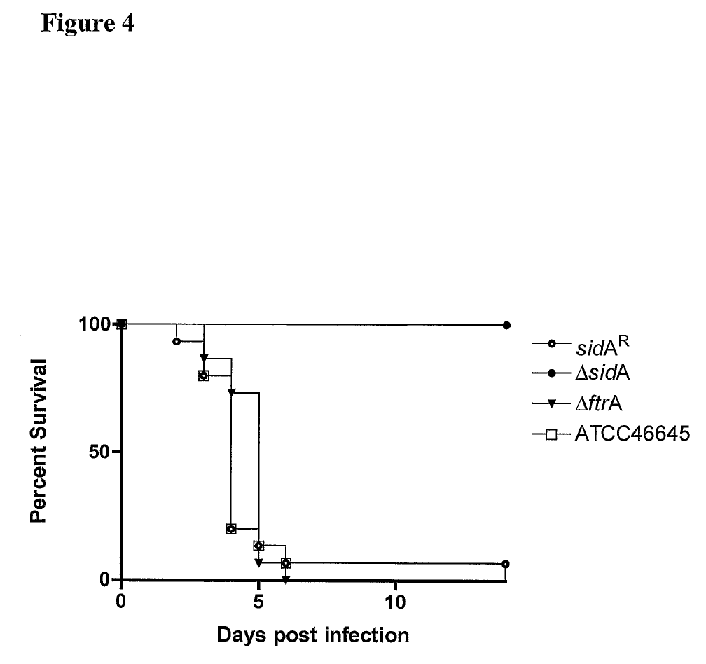

To test the impact of sidA and ftrA in a murine model of systemic aspergillosis, we generated the sidA-deficient mutant ΔsidA and the ftrA-deficient mutant ΔftrA in A. fumigatus ATCC46645 by replacement with the hph marker instead of using pyrG. This procedure was required because it has been suggested recently that the genomic location of the pyrG ortholog URA3 contributes to the severity of murine systemic candidiasis, which confounds interpretation of the role of the gene of interest in pathogenicity (Staab, Trends. Microbiol. (2003), 11, 69-73), and, as URA3 in C. albicans, pyrG is an essential gene of A. fumigatus and required for virulence (D'Enfert (1996), Infect. Immun. 64, 4401-4405). In all tests described, ΔsidA and ΔftrA displayed the same features as the respective mutants generated in CEA17 (FIG. 1). The ΔsidA strain was in contrast to a ftrA-deficient strain absolutely avirulent (FIG. 4). The wild-type growth phenotype siderophore production (FIG. 1) and virulence of A. fumigatus strain ΔsidA^{r}, which was generated by reconstitution of sidA by a silently mutated version in ΔsidA, demonstrated that the loss of virulence of ΔsidA is solely due to SidA-deficiency. Remarkably, sidA is the first A. fumigatus gene described, which is not essential for survival in standard growth media but nevertheless is essential for virulence in a murine model. Due to the fact that mammals lack a similar system, SidA—and possibly the siderophore system in general represents an attractive target for development of therapies against A. fumigatus and likely also other siderophore-producing fungi. In this respect it is important to note that several pathogenic fungi produce hydroxamate-type siderophores (Howard (1999), loc. cit.) and data base searches (http://www.ncbi.nlm.nih.gov/blast/; http://www.ncbi.nlm.nih.gov/sutils/genom_tree.cgi) identified sidA orthologs in the genomes of numerous fungi, including Aureobasidium pullulans, Neurospora crassa, Aspergillus oryzae, Schizosaccharomyces pombe, U. maydis, Gibberella zeae, and Coccidioides posadasii.

### EXAMPLE 15

### Screening Methods for Inhibitors of Siderophore Biosynthesis of Aspergillus fumigatus

1) Discrimination of siderophore production versus non-production by inhibition of growth of siderophore non-producing strains of A. fumigatus in the presence of 5% blood.

Microtiter plate wells containing liquid or solid Aspergillus minimal medium (Pontecorvo, Adv. Genet. 5 (1953), 141-238, Oberegger, Mol. Microbiol. 41 (2001), 1077-1089) plus 5% sheep blood with and without different inhibitors are inoculated with 10²-10⁴ conidia of A. fumigatus, incubated for 24-72 h at 37° C. and growth is scored. Lack of siderophore production causes inhibition of growth. Growth inhibition can be determined, e.g., by a spectrophotometrical (measuring the optical density at 620 nm with a microtiter plate reader), quantitative, automated assay (Broekaert, FEMS Microbiol. Lett. 69 (1990), 55-60; Ludwig, FEMS Microbiol. Letters 69: (1990), 61-66). Specific inhibition of siderophore biosynthesis is indicated if the inhibitor causes less inhibition of growth on media without blood or if the inhibition can be antagonized by supplementation with siderophores, e.g. 10 μM ferricrocin or 10 μM triacetylfusarinine C. Inhibition of siderophore biosynthesis can also be determined by the CAS-assay, HPLC-analysis or mass spectroscopy (see below).

2) Discrimination of siderophore production versus non-production by inhibition of growth of siderophore non-producing strains of A. fumigatus in the presence of 200 μM ferrous iron chelators like bathophenanthroline-disulfonic acid (BPS) (due to inhibition of the reductive iron uptake system).

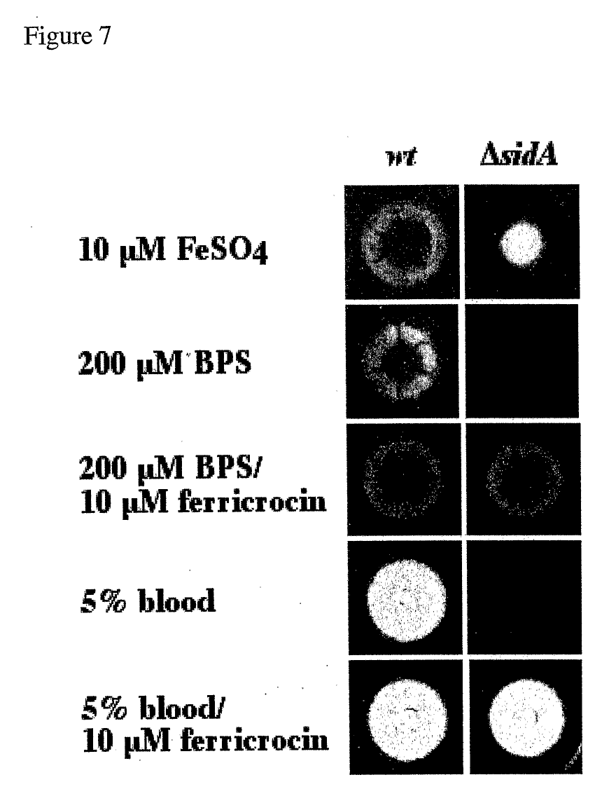

Microtiter plate wells containing liquid or solid Aspergillus minimal medium (Pontecorvo, Adv. Genet. 5 (1953), 141-238; Oberegger, Mol. Microbiol. 41 (2001), 1077-1089) plus 200 μM BPS with and without different inhibitors are inoculated with 10²-10⁴ conidia of A. fumigatus, incubated for 24-72 h at 37° C. and growth is scored. Lack of siderophore production causes inhibition of growth. Growth inhibition can be determined, e.g., by an spectrophotometrical (measuring the optical density at 620 nm with a microtiter plate reader), quantitative, automated assay (Broekaert, FEMS Microbiol. Lett. 69 (1990), 55-60; Ludwig, FEMS Microbiol. Letters 69: (1990), 61-66). Specific inhibition of siderophore biosynthesis is indicated if the inhibitor causes less inhibition of growth on media without BPS or if the inhibition can be antagonized by supplementation with siderophores, e.g. 10 μM ferricrocin or 10 μM triacetylfusarinine C (see FIG. 7). Inhibition of siderophore biosynthesis can also be determined by the CAS-assay, HPLC-analysis or mass spectroscopy (see below).

3) Detection of siderophores by a simple colour assay, e.g. the chrome azurol S (CAS) assay (Payne, Metods Enzymol. 235 (1994) 329-344), or reversed phase HPLC (Konetschny-Rapp Biol. Met. 1 (1988), 9-17; Oberegger, Mol. Microbiol. 41 (2001), 1077-1089), or mass spectroscopy.

Microtiter plate wells containing liquid or solid Aspergillus minimal medium (Pontecorvo, Adv. Genet. 5 (1953), 141-238; Oberegger, Mol. Microbiol. 41 (2001), 1077-1089) lacking iron with and without inhibitors are inoculated with 10²-10⁴ conidia of A. fumigatus, incubated for 24-72 h at 37° C. For detection of siderophores by the CAS-assay an equal volume of blue CAS-solution is added. In the presence of siderophores the blue CAS solution turns red. Therefore the inhibition of siderophore biosynthesis is indicated by blue colour. The type of siderophores produced can be monitored by reversed-phase HPLC or mass spectroscopy.

### EXAMPLE 16

### Screening Test for Inhibitors of Ornithine Mono Oxygenase

OMO is purified from cellular extracts of A. fumigatus grown during iron starvation or purified from E. coli expressing the A. fumigatus OMO-encoding gene. L-Ornithine-N⁵-oxygenase enzyme activity in the presence and absence of inhibitors is determined (Mei, Proc. Natl. Acad. Sci. 90 (1993), 903-907; Zhou, Mol. Gen. Genet. 259 (1998), 532-540). Briefly, OMO is incubated at 30° C. for 2 h in 0.1 mM potassium phosphate pH 8.0, 0.5 mM NADPH, 5 μM FAD, and 1.5 mM L-ornithine. The reaction is stopped by addition of perchloric acid to a final concentration of 66 mM. Samples are centrifuged and the supernatants are subject to the iodine oxidation test (Tomlinson, Anal. Biochem. 44 (1971), 670-679). Subsequently, the samples are briefly zentrifuged to remove denatured protein precipitates, and the absorbance at 520 nm is determined.

### EXAMPLE 17

### Screening Test for Inhibitors of Siderophore Biosynthesis in Aspergillus nidulans

Inhibition of siderophore biosynthesis in A. nidulans causes inhibition of growth in standard media, e.g. AMM (Eisendle, Mol. Microbiol. (2003), 359-375). Specific inhibition of siderophore biosynthesis is indicated if the activity of the inhibitor is antagonized by supplementation with siderophores, e.g. 10 μM ferricrocin or 10 μM triacetylfusarinine C.

### EXAMPLE 18

### Northern Analysis of at1, sidD, at2 Expression in A. fumigatus ATCC46645-Expression of sidD, at1, at2 is Induced by Iron Starvation

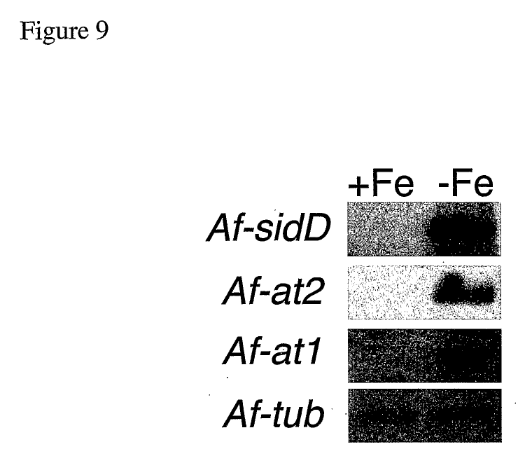

Total RNA isolation and Northern analysis was performed as described in Example 1. The hybridization probes used in this study were generated by PCR using primer 5′-TTGGCGAGAGGAGAGATG and 5′-TACGATGGGTGGTCAGAG for sidD, 5′-CCTCATCCCTATCTCACC and 5′-AGTTTTGAGCGAGAGGGG for at1, and 5′-ACAATCAAGGCTCAGCCC and 5′-ACT TCGAGTCATGCTGGG for at2 (FIG. 9).

### EXAMPLE 19

### Disruption of at1

To construct an at1 deletion mutant, the two fragments flanking the deleted region of at1 were amplified by PCR using the primers 5′-GCAGATCGATAACTTAGACGGCCTCCAC and 5′-CTCGGAGCTCCTTTGAGTCGCCATCGC for flanking region A (1.2 kb), and, 5′-CTGGAATCTAGAGATCGGATGGCGTGGG and 5′-CTGCAAGCTTATGGGGTTGGCACTAAGC for flanking region B (1.4 kb). Two primers contained the add-on restriction sites SacI and XbaI, respectively (add-on restriction sites are underlined). Subsequent to gel-purification, the fragments were digested with SacI and XbaI, respectively The hph selection marker was released from plasmid pAN7-1 (Punt, Gene 56 (1987), 117-124) by digestion with SacI and XbaI, and ligated with the two flanking regions A and B described above. For generation of Δat1, the split-marker recombination according to deHoogt (Biotechniques 28 (2000), 1112-1116) was used. Therefore, two overlapping fragments were amplified from the ligation product using primers 5′-AATGCTCGTACTCCCTCG and 5′-GAAGATGTTGGCGACCTC for fragment C (2.7-kb) and primers 5′-GGCTTGGCCTAATACCTG and 5′-GAGAGCCTGACCTATTGC for fragment D (2.8-kb). Subsequently ATCC46645 was transformed simultaneously with the overlapping fragments C and D. In the generated mutant allele of Δat1-hph the deleted region encompasses the region encoding amino acids 5-451 of at1.

### EXAMPLE 20

### Southern Blot Analysis of Δat1

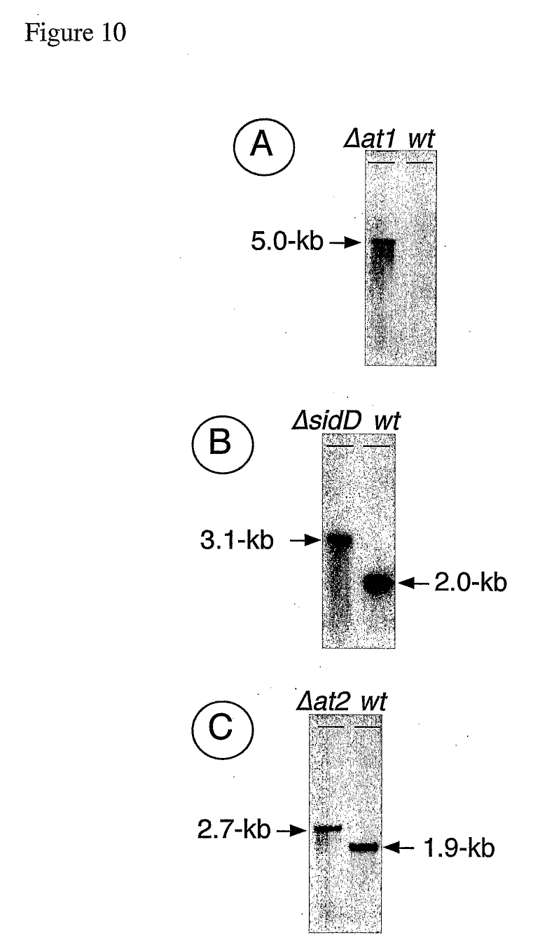

For Southern analysis genomic DNA was digested with NarI, subject to electrophoresis, blotted onto nylon membrane and hybridized with a probe amplified with 5′-CCATACTCCATCCTTCCC and 5′-TTCTGCGGGCGATTTGTG by techniques known in the art. Consistent with the genotype, the probe detected a fragment in the length of 5.0-kb in Δat1 (FIG. 10).

### EXAMPLE 21

### Disruption of sidD

To generate a ΔsidD allele, a 5.1-kb fragment was amplified using primers 5′-GGAGGCGCCGTTGTTTCCCTCGAC (containing an add-on NarI restriction site) and 5′-TTTCCGCAGATGTATCGAGTC, subsequently subcloned into pGEM-T (Promega), sequenced and termed pSIDD. An internal 2.4-kb BglII-XbaI fragment was replaced by a 3.9-kb BglII-XbaI fragment of vector pAN7-1 (Punt, Gene 56 (1987), 117-124) containing the hygromycin B (hph) selection marker. For transformation of ATCC46645, the gel-purified 6.5-kb NarI fragment was used. In the generated mutant allele of ΔsidD-hph the deleted region encompasses the region encoding amino acids 305-1120 of sidD.

### EXAMPLE 22

### Southern Blot Analysis of ΔsidD

For Southern analysis genomic DNA was digested with PvuII, subject to electrophoresis, blotted onto nylon membrane and hybridized with a probe amplified with 5′-CAGAAGTTCCCCGACAAG and 5′-AGTCGTTTACCCAGAATG by techniques known in the art. Consistent with the genotypes, the probe detected fragments in the length of 2.0-kb and 3.1-kb in ATCC46645 and ΔsidD, respectively (FIG. 10).

### EXAMPLE 23

### Disruption of at2

To construct an at2 deletion mutant, the two fragments flanking the deleted region of at2 were amplified by PCR using the primers 5′-AAGGATCGATGGAATATGACGAACCCGC, 5′-ACTCTCGAGGCATCACCCAACATCCTC for flanking region A (1.7 kb), 5′-GATATTTTAAATACCTCATGGCGTGCAAC and 5′-GTGTGCGGCCGCGTGTACCTCTTGCTTCCC for flanking region B (1.3 kb). Two primers contained the add-on restriction sites XhoI and NotI, respectively (add-on restriction sites are underlined). Subsequent to gel-purification, the fragments were digested with XhoI and NotI, respectively The hph selection marker (also containing a thymidine kinase) was released from plasmid pHYTK (Sachs, Nucleic Acids Res. 25 (1997), 2389-2395) by digestion with XhoI and NotI, and ligated with the two flanking regions A and B described above. For generation of Δat2, the split-marker recombination according to deHoogt (Biotechniques 28 (2000), 1112-1116) was used. Therefore, two overlapping fragments were amplified from the ligation product using primers 5′-GCCCACCAAACTGTCTTC and 5′-GAAGATGTTGGCGACCTC for fragment C (3.1-kb) and primers 5′-GCGTATGGAGCCAAGAGA and 5′-GAGAGCCTGACCTATTGC for fragment D (3.2-kb). Subsequently ATCC46645 was transformed simultaneously with the overlapping fragments C and D. In the generated mutant allele of Δat2-hph the deleted region encompasses the entire coding region, 117 bp of the 3′-downstream and 113 bp of the 5′-upstream region of at2.

### EXAMPLE 24

### Southern Blot Analysis of Δat2

For Southern analysis genomic DNA was digested with NruI, subject to electrophoresis, blotted onto nylon membrane and hybridized with a probe amplified with 5′-GTGTGCGGCCGCGTGTACCTCTTGCTTCCC and 5′-GCGTATGGAGCCAAGAGA by techniques known in the art. Consistent with the genotypes, the probe detected fragments in the length of 1.9-kb and 2.7-kb for ATCC46645 and Δat2, respectively (FIG. 10).

### EXAMPLE 25

### at1, sidD and at2 are Involved in Biosynthesis of Triacetylfusarinine C, TAFC but not Ferricrocin (FC)

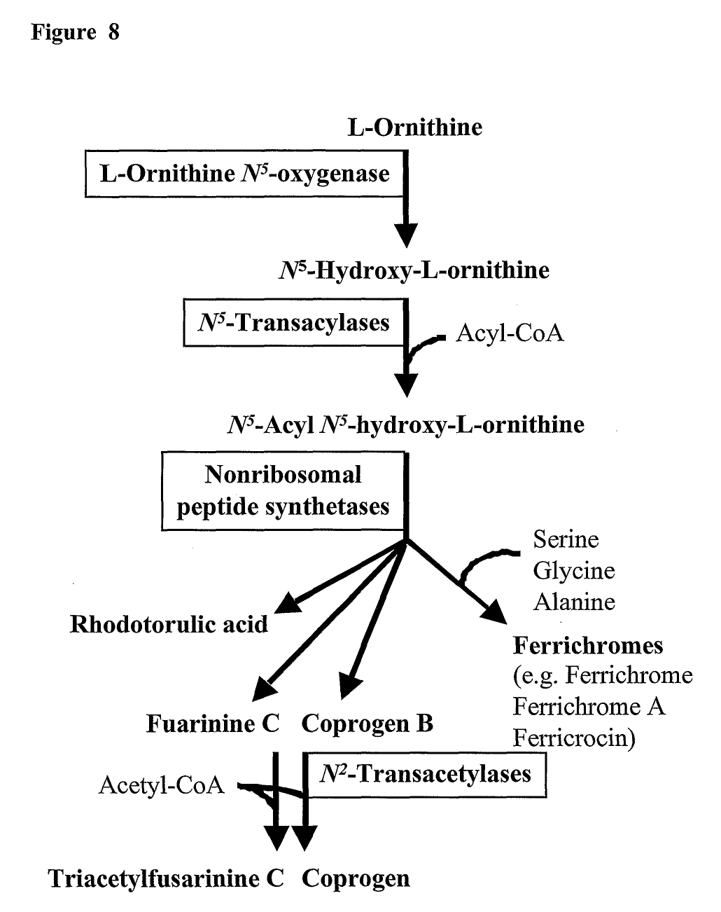

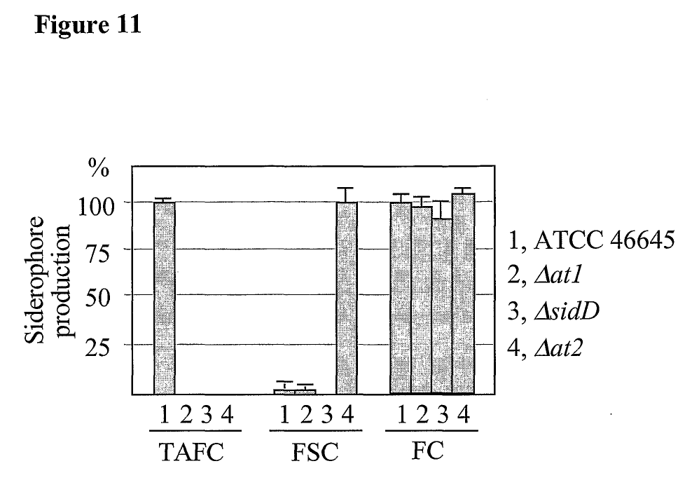

Analysis of siderophore production was performed by Reversed-phase-HPLC according to Oberegger (Mol. Microbiol. 41 (2001), 1077-1089) as performed for analysis of the sidA mutant. Deletion of at1, sidD and at2 resulted in loss of production of TAFC but not FC. In Δat2 the precursor of TAFC, Fusarinine C (FIG. 8), was detected in amounts similar to TAFC in the wild type (FIG. 11).

### EXAMPLE 26

### Deletion of at1 and sidD but not at2 Results in a Decrease of the Radial Growth Rate on Blood Agar and During Iron Depleted Conditions in the Presence of Bathophenantroline-Disulfonic Acid

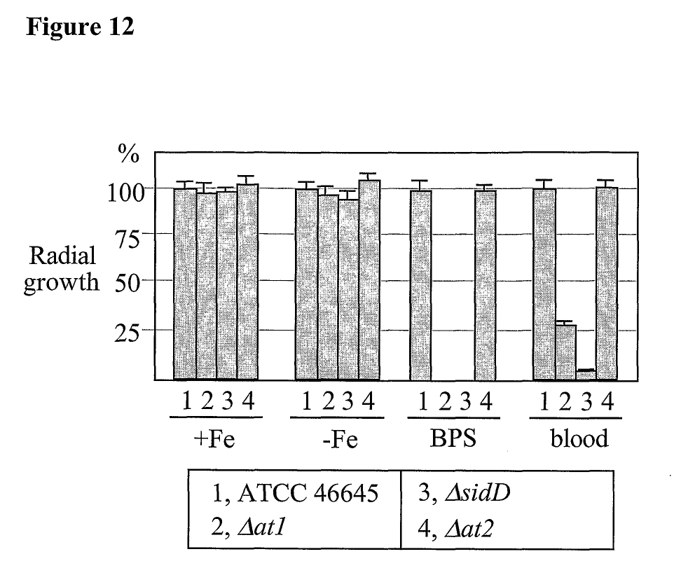

The growth rates of the strains were tested during iron-replete conditions, iron depleted conditions, iron depleted conditions in the presence of bathophenantroline-disulfonic acid and on blood agar as described in FIG. 3 and radial growth was scored at 48 h. Δat2 displayed a wild type growth rate during all conditions (FIG. 12). In contrast, Δat1 and ΔsidD showed a decreased growth rate during iron depleted conditions in the presence of bathophenantroline-disulfonic acid and on blood agar. Bathophenantroline-disulfonic acid is an inhibitor of the reductive iron assimilatory system and the iron contained in blood agar cannot be readily used by the reductive iron assimilatory system. Therefore, these data are consistent with the siderophore analysis (Example 25); i.e. Δat1 and ΔsidD do not produce a functional siderophore, which could compensate the blocked reductive iron assimilation-dependent iron uptake, whereas Δat2 does produce a functional siderophore.

### EXAMPLE 27

### at1, sidD and at2 are Essential for Full Virulence

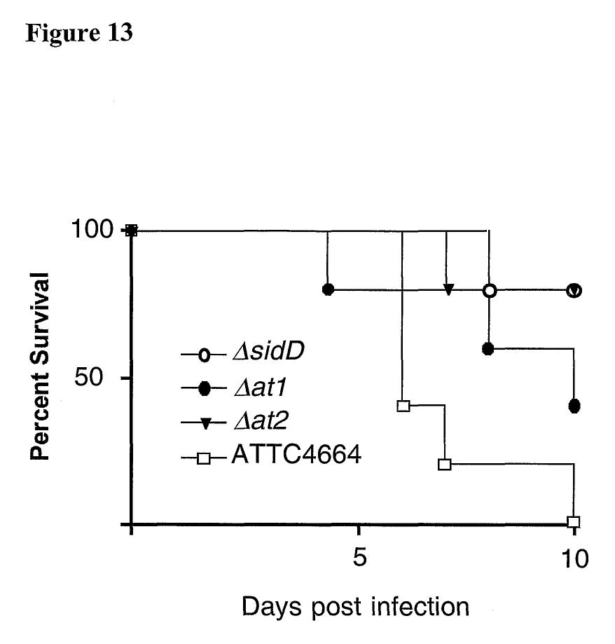

Δat1, ΔsidD and Δat2 showed attenuated virulence in a mouse model for pulmonary aspergillosis (FIG. 13)—the virulence assay is described in Example 13. Taken together, the data show that triacetylfusarinine C production is crucial for virulence of A. fumigatus. Remarkably, in Δat2 production of Fusarinine C, which fully replaces triactylfusarinine during saprophytic growth cannot replace triacetylfusarinine C function during pathogenic growth.

### EXAMPLE 28

### Addendum to Example 15

### Screening Methods for Inhibitors of Siderophore Biosynthesis of Aspergillus fumigatus

For screening of inhibitors of At1 and SidD the methods 1), 2) and 3) of Example 15 can be applied as the Δat1 and ΔsidD mutants display a decreased growth rate on blood agar and in the presence of bathophenanthroline-disulfonic acid (FIG. 12). For screening of inhibitors of At2 lack of TAFC production by reversed phase HPLC analysis as described under 3/Example 28 can be applied.

### EXAMPLE 29

### Screening Inhibitors of At2

AT2 is purified from cellular extracts of A. fumigatus grown during iron starvation or purified from E. coli expressing the A. fumigatus AT2-encoding gene. AT2 activity in the presence and absence of inhibitors is determined. Briefly, AT2 is incubated at 30° C. for 0.5 h in 0.1 mM potassium phosphate pH 8.0, 0.1 μCi of [1-¹⁴C]acetyl-CoA (55 mCi/mmol) and 0.1 mM fusarinine C in a final volume of 200 μl. Subsequently, synthesized triacetylfusarinine C is separated from fusarinine C by extraction into chloroform and quantified by scintillation counting.

### EXAMPLE 30

### Disruption of Rac1

To construct an race deletion mutant, the two fragments flanking the deleted region of rac1 were amplified by PCR using the primers 5′-AAGATCGATCGTCGGGTCCATTAGTAC, 5′-ACGGCGGCCGCTGGAGAAGCGAAAGCCAC for flanking region A (1.7 kb), 5′-AGCTTTAAAAGGTAATTGCGGTGGTGC and 5′-AGGGGATCCAAACGAGACGAGGCATCC for flanking region B (1.3 kb). Two primers contained the add-on restriction sites BamHI and NotI, respectively (add-on restriction sites are underlined). Subsequent to gel-purification, the fragments were digested with BamHI and NotI, respectively. The hph selection marker (also containing a thymidine kinase) was released from plasmid pHYTK (Sachs, Nucleic Acids Res. 25 (1997), 2389-2395) by digestion with BamHI and NotI, and ligated with the two flanking regions A and B described above. For generation of Δrac1, the split-marker recombination according to deHoogt (Biotechniques 28 (2000), 1112-1116) was used. Therefore, two overlapping fragments were amplified from the ligation product using primers 5′-GACATCATGCAGCCCAAC and 5′-GAAGATGTTGGCGACCTC for fragment C (3.1-kb) and primers 5′-GGTGCTCTTCGTTTTGCC and 5′-GAGAGCCTGACCTATTGC for fragment D (3.2-kb). Subsequently ATCC46645 was transformed simultaneously with the overlapping fragments C and D. In the generated mutant allele of Δrac1-hph the deleted region encompasses the region encoding amino acids 17-261 of rac1.

For Southern analysis genomic DNA was digested with XbaI, subject to electrophoresis, blotted onto nylon membrane and hybridized with a probe amplified with 5′-AAGATCGATCGTCGGGTCCATTAGTAC and 5′-ACGGCGGCCGCTGGAGAAGCGAAAGCCAC by techniques known in the art. Consistent with the genotypes, the probe detected fragments in the length of 3.3-kb and 5.5-kb in ATCC46645 and Δrac1, respectively (FIG. 10).

Northern was performed as described in Example 1. The rac1 hybridization probe used in this study was generated by PCR using primers 5′-CACTGTGGCTTTCGCTTC and 5′-CTCCGACCTACAGACAAC.

1. A method for screening inhibitors of fungal siderophore biosynthesis comprising
(a) contacting a cell expressing a fungal siderophore with a compound to be tested;
(b) determining whether said cell is capable of siderophore biosynthesis in the presence of said compound to be tested when compared to a cell not contacted with said compound; and
(c) identifying the compound which inhibits siderophore biosynthesis.

2. The method of claim 1, wherein said cell is a whole cell extract.

3. A method for screening inhibitors of fungal siderophore biosynthesis comprising the steps of
(a) contacting an enzyme involved in siderophore biosynthesis with a compound to be tested;
(b) determining whether said enzyme is functional in the pathway of siderophore biosynthesis in the presence of said compounds to be tested when compared to an enzyme not contacted with said compound; and
(c) identifying the compound which inhibits the enzymatic function involved in siderophore biosynthesis.

4. The method of claim 3, wherein said enzyme involved in siderophore biosynthesis is present in whole cell extracts, is unpurified, is partially purified, is purified or is recombinantly expressed.

5. A method for screening inhibitors of fungal siderophore biosynthesis comprising the steps of
(a) contacting a polynucleotide coding for an enzyme involved in siderophore biosynthesis with a compound to be tested;
(b) determining whether said polynucleotide is expressed in the presence of said compounds to be tested when compared to a second polynucleotide comprising the same nucleotide sequence which is not contacted with said compound; and
(c) identifying the compound which inhibits the functionally expression of the polynucleotide expressing an enzyme involved in siderophore biosynthesis.

6. The method of claim 1, wherein said fungal siderophore biosynthesis is in Aspergillus spec.

7. The method of claim 2, wherein said Aspergillus spec. is Aspergillus fumigatus.

8. The method of claim 1, wherein said siderophore biosynthesis comprises one or more enzymes selected from the group consisting of L-ornithine N5-oxygenase, N5-transacylase, non-ribosomal peptide synthetase, enoyl CoA hydratase and N2-transacetylase and/or fragments thereof.

9. The method of claim 3, wherein said enzyme involved in siderophore biosynthesis is selected from the group consisting of L-ornithine N5-oxygenase, N5-transacylase, non-ribosomal peptide synthetase, enoyl CoA hydratase and N2-transacetylase and/or fragments thereof.

10. The method of claim 8, wherein said L-ornithine N5-oxygenase is encoded by a polynucleotide comprising the sidA gene of Aspergillus fumigatus shown in SEQ ID NO: 1.

11. The method of claim 8, wherein said N5-transacylase is encoded by a polynucleotide comprising a nucleic acid molecule having the nucleotide sequence shown in SEQ ID NO: 3.

12. The method of claim 8, wherein said non-ribosomal peptide synthetase is encoded by a polynucleotide comprising a nucleic acid molecule having the nucleotide sequence shown in SEQ ID NO: 5.

13. The method of claim 8, wherein said enoyl CoA hydratase is encoded by a polynucleotide comprising a nucleic acid molecule having the nucleotide sequence shown in SEQ ID NO: 7.

14. The method of claim 8, wherein said N2-transacetylase is encoded by a polynucleotide comprising a nucleic acid molecule having the nucleotide sequence shown in SEQ ID NO: 9 or SEQ ID NO: 16.

15. The method of claim 1, wherein said siderophore is extracellular.

16. The method of claim 1, wherein said siderophore is intracellular.

17. The method of claim 1, wherein said compound to be tested is of chemical or biological origin.

18. The method of claim 1, wherein said compound to be tested is synthetically, recombinantly and/or chemically produced.

19. The method of claim 1, wherein said screening is high throughput screening (HTS).

20. The method of claim 1, wherein said cell is selected from the group consisting of a mammalian cell, insect cell, amphibian cell, fish cell, fungal cell and bacterial cell.

21. The method of claim 20, wherein said cell harbours one or more polynucleotides operatively linked to expression control sequences capable of expressing one or more of the enzymes selected from the group consisting of L-ornithine N5-oxygenase, N5-transacylase, non-ribosomal peptide synthetase, enoyl CoA hydratase and N2-transacetylase and/or fragments thereof.

22. A method for the production of a pharmaceutical composition comprising the steps of the method of claim 1 and the subsequent step of mixing the compound identified in step (c) with a pharmaceutically acceptable carrier.

23-24. (canceled)

25. A method of preparing a pharmaceutical composition comprising the steps of
(a) identifying a compound which inhibits fungal siderophore biosynthesis; and
(b) formulating said compound with a pharmaceutically acceptable carrier.

26. A pharmaceutical composition comprising an inhibitor of siderophore biosynthesis in Aspergillus species, preferably Aspergillus fumigatus.

27-28. (canceled)

29. A method of treating and/or preventing a disease associated with fungal infection comprising administering a therapeutically effective amount of a pharmaceutical composition comprising an inhibitor of fungal siderophore biosynthesis to a subject suffering from said disorder.

30. (canceled)

31. A pharmaceutical composition comprising an inhibitor of siderophore biosynthesis in Aspergillus species, preferably Aspergillus fumigatus or the method of claim 29, wherein said inhibitor of fungal siderophore biosynthesis is selected from the group consisting of antibodies, aptamers, RNAi, shRNA, siRNA, RNAzymes, ribozymes, antisense DNA, antisense oligonucleotides, antisense RNA, affibodies, trinectins and anticalins.

32. The pharmaceutical composition or method of claim 31, whereby said inhibitor targets a nucleotide sequence as comprised in any one of SEQ ID NOS: 1, 3, 5, 7, 9 or 16 or wherein said inhibitor targets an expression product of any one of SEQ ID NOS: 1, 3, 5, 7, 9 or 16.

33. The pharmaceutical composition or method of claim 31, wherein said target nucleotide sequence comprises the sequence selected from the group consisting of SEQ ID NOS: 72, 75, 78, 81, 84, 87, 90, 93, 96, 99, 102, 105, 108, 111, 114, 117, 120, 123, 126, 129, 132, 135, 138, 141, 144, 147, 150, 153, 156 and 159.


SEQUENCE LISTING (161 sequences)

SEQ ID NO: 1 - DNA, 1506 bp, Aspergillus fumigatus
atggaatctgttgaacggaagtcagaatcgagctacttgggtatgcgcaacatgcagcccgagcagcgtctttctttggatcctccccgcttgaggtcaacaccccaggatgagcttcatgatcttctgtgtgttgggtttggacccgcttccctggccattgccattgctttgcatgacgctctggaccctcgattgaacaagtccgcttccaatatccatgcacagcctaagatctgcttcctggagcgccagaagcaatttgcgtggcactcgggtatgctggtccccggttccaagatgcagatctccttcatcaaggatctcgcaactctccgggacccccgcagcagttttacttttctcaactacctccaccagaagggccgtctgattcacttcactaacctcagcaccttcctgccggctcggctggagttcgaggactacatgcgttggtgtgcgcaacaattttcggatgtagtggcttacggggaagaggtggtcgaagtgattcccgggaagtctgatcccagcagctcggtggttgacttcttcactgttcggtcgcgcaacgttgagacgggcgagatcagtgccaggaggacccgcaaggtcgttattgcaatcggaggcactgcaaagatgccatccggactgccccaggatccccggattatacactcgtccaagtactgcacaacgctgccggccctgttgaaggacaagtcgaagccttacaacattgccgttctgggcagtggtcagagtgctgcggagattttccatgaccttcaaaagagatatcccaactcacgaacaacgctgattatgcgggattctgcaatgcggcccagtgacgactcgccttttgtgaatgagatcttcaaccccgagcgagtcgacaagttctacagccaatctgccgcagaacgccagcgctctctcctcgccgacaaggctaccaactacagtgttgtccgcctggagttgattgaggagatctataacgacatgtacctgcagagggtgaagaaccctgacgaaactcagtggcagcatcgcatccttccggaacgcaagatcacacgggtcgagcaccatggtcctcagagtcgaatgcgcatccacctcaagtcatccaagcccgagtcggaaggcgcagcaaacgacgtcaaagaaacgctggaagtcgacgctctcatggtggctacaggctacaatcgcaacgcgcacgagcgactcctgagcaaggttcaacacttgagacctacgggacaggatcagtggaagccccaccgagattaccgggttgagatggacccgagcaaggtcagctcagaagccggcatctggcttcaaggttgtaatgagcggacgcatggccttagtgacagtttgctgtcggtactggctgttcgtggtggcgagatggtccaatcgattttcggagagcagctggaaagagcggcggtacagggccaccagctacgagccatgctgtaa

SEQ ID NO: 2 - protein, 501 aa, Aspergillus fumigatus [sequence omitted]

SEQ ID NO: 3 - DNA, 1065 bp, Aspergillus fumigatus
atgtactcgaaattctggcccaaaggcggtctcccaggcattctacaccactacacagaaaccctcgtcacatttgaatacaccacaaccaccacccgcaaaccacacagtctcctcttcgttggcggtctcggcgatggcctagcaacaacatcctacctggcggatctagctcacgccctccaaccaacagaatggtccctcttcaccctgaccctgacctcctcgtaccaatcctgggggctggggcacctcgaccgagacaccaacgaaatcgcacagtgcctgaagtacatcaaggagtacaaaaccgaaaaattcggcggcagcgctagcagcgggaagatcgtccttatgggccactcaacaggtagccagtgcgtgcttcactacctgtcccgaccaaatccgcacacacacaccccggccttcgacccctacctggagcatgttgagcgaatgccgctcgacggggcgatcatgcaggcgcccgtgtcggaccgggaagcgatccagtgggtccttgccgaggggttgggagataggacgccggcggagatccggcctgtgtttgagaagttgactagcatggcgagggaggctgctcgcgacgcggacgctggcactgatgtcctgctgccgttggcgatgacttcgctggtatacccggctcatacgccgctgagtgcgcggcggtttctgagtctgacgagtcctgagagtccagagtcgccgagtgaggatgatctatttagttcggatctgagcgatgagcagttggggaagacgttcgggatgatcagggagcagggtctcctgagggggaaactgatggtgttgttttctggggcggaccagtcggttccggcctgggttgacaaggacacgttactgtctaggtggaggaatgccacagatcataatggggaagccgcgatatgggacgaaaacagcggtatcataccgaatgcgtctcatgcattgagtaatgatgatcaagctgagcctcggaactttctggtcaacaaggtactaggatatctttcggctttagtaaaagcttag

SEQ ID NO: 4 - protein, 354 aa, Aspergillus fumigatus
MYSKFWPKGGLPGILHHYTETLVTFEYTTTTTRKPHSLLFVGGLGDGLATTSYLADLAHALQPTEWSLFTLTLTSSYQSWGLGHLDRDTNEIAQCLKYIKEYKTEKFGGSASSGKIVLMGHSTGSQCVLHYLSRPNPHTHTPAFDPYLEHVERMPLDGAIMQAPVSDREAIQWVLAEGLGDRTPAEIRPVFEKLTSMAREAARDADAGTDVLLPLAMTSLVYPAHTPLSARRFLSLTSPESPESPSEDDLFSSDLSDEQLGKTFGMIREQGLLRGKLMVLFSGADQSVPAWVDKDTLLSRWRNATDHNGEAAIWDENSGIIPNASHALSNDDQAEPRNFLVNKVLGYLSALVKA

SEQ ID NO: 5 - DNA, 6252 bp, Aspergillus fumigatus
atgggttcaatacagcaagatgacgttcacaatcagatagaccactgcaatcagtcagatgacttaccggcggcccggttgaattgcaatgacgtcgaattattcgaggtggctggcctcgcatgcgacgagacctcgagtcccacaggcatgagagacgaaatggtacttttatcgtggctcattgcgctcctgcggacacgagaaggcggccagattcggtacgaatgggcatataggtatccagaagaggagccggtacctagatgtctggctatgaatgaagtagtggcggggttacaaagtagtgtcaaggaaactgcagcagctgtgtctcggcatatttccgcagatgtatcgagtccgccggcgccagcatctctgcttctgagcactagctccctttctcagacatcagacgaagcaaaagatgaagggttacttcacctagaaatcgctttcgagaatgggctgtgcaagattcgtccgacatggcactccgagaatatgttgccgtttacagtgacgcgatacgccaggaccctcatcgacacggttaggctgtgcatttccaattgcgatgccgccatccaagactgccttcggcctaccgcatacgatctcgacgagatatggcgctggaaccacaacctgcctcccacgtacaatttctgcatgcacgaaataatctccgaccaggctcagaagttccccgacaaggaagcaatcgcatcctgggacggcagcctgacttacagacagattgatcagtattcatcattcgtcgcgcgctcacttataggcatgggagttgggctgcatgatgtcttaccagtctgctttgagaaatcccgatggacgattgtcgcggttctggccgtaatgaaagctggcgcgacgtttgtactaatggacccgacgcttccacttgcacgattgcagaacatggcccagcaggtcggcgcgaagatgatggtttcatctcggggccagtacaacctggcgacggagatcatacccaatgcaaacgtgctcgtcgtggaggaaaacacgttcagcagtctgtccgccgaacagaacggcgagcccctgccaacggttccctcctcggccctgatgtacatgatctttacatccggcagcacaggtactccgaaaggagtcaaaatctcccacgaaacctacaccagtagcgccattccccgggccaatgcggtaggttacacggaggattcccgcgttctcgacttcgcctcctacgccttcgacgtcagcatcgacagtatgctgctgaccctgggcaacggcggctgtctgtgcattccgtccgacgaagaccgcctgaacgacatcaatggcgtgatacggcgaatgaaagtcaactatgcgggcctgacgccttcggtagcccgtattctagacgcagatgtaatatcctcgctcagtggtcttggcctcggaggagaggccgtctcagcgagagatgtcaatctctggggtcaggacaccaggattattatcggctacgggccctgcgaatgcacgattggatgcacagtgaatagtagcgcggcaacgggaagagactatatctccattggccctggtaatggggcagtaatctggatagtcgatcctaacgaccatgaatctctcgtgcctctcggcgcggtaggagagctgctggtagaaggtcccatagtagggcagggctacttgaatgatccggagaaaactgcagcagcgttcattgaagacccctcatggttggttgcgggccacgaaggttaccccggtcgtcggggccgtttgtacaagaccggtgatctcggtcgatatgacccagatgggtcaggcggtatcgtgttcgtggggcgcaaggatacccaggttaaattgcgaggacaacgggtagagctcggggagatcgaaagccaattgagagccagactgccctcggagacgactgtgatcgccgaggtgattgtaccccaaggatctgggggacaaccgacgctggtggcatttgttgctgcccagacgaccaagggacatgatcataccggactcgaggcggcagagcttcccgatgagcttcgaagggcgctgtcagaagccgatgcggagctggcaaaggtattgcctcgatacatggtgccaacagcgtatattcctgtcaaccacatccctacgctgatttcaggcaaaacagaccgcaagagactgaggcagttcggcgcaaccgttgacctgcgccagctggaccaggatgctacgaacaccgcagctcgggagctgagtgatctggagcgtcgcttgcgacaggcatggagccagacgttgaagctccaagcgtgttctattcgtctacaggacaacttcttcgctcttggtggagactccctcacagccatgaagctggtctctgtatgtcggtcccagggtctcgacctcagtgtcaccagcatgtttagcaatcctaccctgtcagccatggccagcgttgtccgtatttgcgacgtcgacgtgcaaaggacagtcccggcattctcgatgatcacatcagatatgaacagtgcctgtgtagaggcagcagagccctgcggagttggcccagcagatatcgaggacatctacccctgtacgccaacacaggagtctcttttcactttctcgctcaagtcagtcaagccctacgtggcacagagagtcctatgtatcccgtctcatatcgacctcaacgcatggaggaaggcatgggaggatgtggtcgccgcactccccatcctacggacacgagtagctcagctgcaagagcctggccttcagcaagttgtactcaagaacagcatttcctggacgcaagcctcagatctagcggaatacctagagaacgaccgaacccaaaagatgaatctcggagagagtctggcccgttacgccatcgtcgaagactcagccgacggcaagcgatacatggtttggactattcaccatgtgttgtacgatgggtggtcagagccgattatcctcaagcaagtcagcgacgccctgcaagggcagccagtcgaggtaaaggcgcagatgcgagactttgtcagattcgtgcgagactcggatgacgctgcagtgcaggaattctggcggcgcgagctgaaaggtgccgtcgggccccaattcccacgtctaccctcaagagacttcatgcctaccccggacgccctagtcgagcgtcaagtctctcttgatactagctccggatcaccgtttaccatggcgacattgattcgcggcgcctgggcgctcgtcgcgtcccaatatactggaagcgacgatatcgtgttcggcgagaccctcacaggtcgcgacatcccactacctggagtcgaaagcattgttggaccattaattgcgacggtccccatccgcgtgcgcattctccggggaagtactgtggagtcctacctccaagccgtgcagcagagtgttttggcccgtaccccgtaccagcatttgggtatgcagaacatccgcaaggttagccaagatgcgcaacacgcgtgcgagactggtacgggtctggtcattcagcctgaaccagagtatgtcggcagcgaactgggcgttgagagaggggacgtggttcttgaagcgctgcatttcaacccgtatccgctgatgctggcttgcgggattcgcaagggtggtttccgggtttgtgcaagctttgacagcagcttgattgagaccaggcagatggaacgaatgcttgcccagcttgagaccgcgtgctggcagttgagtcaaggtctttcccggaaggtcgatgagatctcgtgtctccccgaagccgagttgaaccagatctggcagtggaatcggagtccacctttgtctctggacgaaacaaccagtcggttgcgtgcgaatgcaagcacaaagcctggttcaagctatcctcccgcggttgttccttgggtttgtagtccacgaaactcatctctcctctcgccaatcggctgtgttggcgagctctggcttgaaggggcccttctttcgggcgacacagttgattcacctgcctggcttgtggctggaagctctacctgtgctggccgaactggcaaagtgcaagcaaccggcgacatggttcagttgcgagaagacggcagtttagtgtttgttggtcggaaggagaatgttgttcctgttcaagggcatgcagtggacatcacagagattgaaaggcacctcgcagaacatctgccgccaacaatccgtgccgctgctactgtagtgcggtcttcatccgaccaggagctggttatgttcattgagcaaccggctgcggaggaagcctgtattgagctcctatcggagaagcgcgaaatagtgtgcgacgcgccagacaaagccttccagaccacaatttgtgcgacaatcccgggcagtcttgctgcagtgttgaagaagttggacaaatacatgcgagactcgcttccgtcatatatggctccgtccgcttacatcgtggtagagaaactgcctaatacgatggacgatatcgaccacaatttgctcaatcagatcgcctctcaggtcactccacaaattctcaacgagcttcgcgatgggttgagcaacgcttggaccaaggctacggctccaaaccatctatcagcttcagaaagcattctgcggtctgcatgggccaaagtgcttcgtgtcgatccggagcagatcgacgtggacgacaatttcttccggcgcggtggtgattccgtacttgcaatgaagcttgtgtcgagtctccgagcgcaaggctacagtttgtccgtggctgatatcttccggcacatgcgtctcagcgacgctgccagagtgatgaaggtggatgaacgctcaacagagaagatcaactcatatcagccgttttcgatgctccgtctcccagacgttcagcagttccttgccaacattgtccgaccacagctaggggaccaacactggcccattcgagacgtgttgccggtgaccgactcccaggatatggatatccgtgcaaccattcagccgccgaggacgtcaatccagtatacaatgctgtactttgacaacagcgttgacagagagcggcttttccgttcgtgcagcgacctggttaagacgcacgagattctccgtaccgtcttcatatcacacgaatccagcttcttgcaggttgtcttgaatgagttggaaattcctgtccgcgcacacaagactgacaagcagctcgatcaatacgtcgccagcctcttcagggaggacattgaatccaacttccagttaggatgtccattcctccgacttttctacgtcgagggaaacaacggcgaatcctgcctcgtcatcggcttatcccacgcccaatacgatggcgtctccctgcccagactcctccaagacctcgacgccctatacaccggcacccagctcgccaccttctccccattctccttgtacatggcccagaccagtgaagaggcaattcaaaacaaggcagcggcctactggcgcaacctcctctccagctcctcactctcgaccctcgacggaccctcctctgatcccaccgacaaagcaatcttccacactcgcccggtcaacatccatcccctcaaggagatcaccaccgcaaacctcctcacagcagcctgggcgatggtcctcgctcgccgtctccaaaccccagacgtcaccttcggcagcgtcacctccggccgcaccctcgacattcccaacgccgaaaacttcatgggcccctgctaccagctcacccccgtccgggtccccttccatcccgactggaccgcaagcgacctgctgaacttcgtccagacacagtctgccgagtctgcggcgcatgacttcctcggttttgagaagatcgctaagctcgcggggtgggcctcaggaaggcaaggctttgattcgatcgtgcatcatcaggactgggaggatttcgatatgatgccgtttggcggagggtcttgtcgggtggacattgcgaatccgcatggggatgcggcgtatccggtcaaggctgtttcgtttgtgaaggagggagagatacatgttggtgtggtctgtagtgagagagatgtgatgtttgtagacgaggttctcggggaattggctgctgcggttgtggagttggctggtcagagtacagaggtgttgctggatagcaagttgtttagtggacagtga

SEQ ID NO: 6 - protein, 2083 aa, Aspergillus fumigatus
MGSIQQDDVHNQIDHCNQSDDLPAARLNCNDVELFEVAGLACDETSSPTGMRDEMVLLSWLIALLRTREGGQIRYEWAYRYPEEEPVPRCLAMNEVVAGLQSSVKETAAAVSRHISADVSSPPAPASLLLSTSSLSQTSDEAKDEGLLHLEIAFENGLCKIRPTWHSENMLPFTVTRYARTLIDTVRLCISNCDAAIQDCLRPTAYDLDEIWRWNHNLPPTYNFCMHEIISDQAQKFPDKEAIASWDGSLTYRQIDQYSSFVARSLIGMGVGLHDVLPVCFEKSRWTIVAVLAVMKAGATFVLMDPTLPLARLQNMAQQVGAKMMVSSRGQYNLATEIIPNANVLVVEENTFSSLSAEQNGEPLPTVPSSALMYMIFTSGSTGTPKGVKISHETYTSSAIPRANAVGYTEDSRVLDFASYAFDVSIDSMLLTLGNGGCLCIPSDEDRLNDINGVIRRMKVNYAGLTPSVARILDADVISSLSGLGLGGEAVSARDVNLWGQDTRIIIGYGPCECTIGCTVNSSAATGRDYISIGPGNGAVIWIVDPNDHESLVPLGAVGELLVEGPIVGQGYLNDPEKTAAAFIEDPSWLVAGHEGYPGRRGRLYKTGDLGRYDPDGSGGIVFVGRKDTQVKLRGQRVELGEIESQLRARLPSETTVIAEVIVPQGSGGQPTLVAFVAAQTTKGHDHTGLEAAELPDELRRALSEADAELAKVLPRYMVPTAYIPVNHIPTLISGKTDRKRLRQFGATVDLRQLDQDATNTAARELSDLERRLRQAWSQTLKLQACSIRLQDNFFALGGDSLTAMKLVSVCRSQGLDLSVTSMFSNPTLSAMASVVRICDVDVQRTVPAFSMITSDMNSACVEAAEPCGVGPADIEDIYPCTPTQESLFTFSLKSVKPYVAQRVLCIPSHIDLNAWRKAWEDVVAALPILRTRVAQLQEPGLQQVVLKNSISWTQASDLAEYLENDRTQKMNLGESLARYAIVEDSADGKRYMVWTIHHVLYDGWSEPIILKQVSDALQGQPVEVKAQMRDFVRFVRDSDDAAVQEFWRRELKGAVGPQFPRLPSRDFMPTPDALVERQVSLDTSSGSPFTMATLIRGAWALVASQYTGSDDIVFGETLTGRDIPLPGVESIVGPLIATVPIRVRILRGSTVESYLQAVQQSVLARTPYQHLGMQNIRKVSQDAQHACETGTGLVIQPEPEYVGSELGVERGDVVLEALHFNPYPLMLACGIRKGGFRVCASFDSSLIETRQMERMLAQLETACWQLSQGLSRKVDEISCLPEAELNQIWQWNRSPPLSLDETTSRLRANASTKPGSSYPPAVVPWVCSPRNSSLLSPIGCVGELWLEGALLSGDTVDSPAWLVAGSSTCAGRTGKVQATGDMVQLREDGSLVFVGRKENVVPVQGHAVDITEIERHLAEHLPPTIRAAATVVRSSSDQELVMFIEQPAAEEACIELLSEKREIVCDAPDKAFQTTICATIPGSLAAVLKKLDKYMRDSLPSYMAPSAYIVVEKLPNTMDDIDHNLLNQIASQVTPQILNELRDGLSNAWTKATAPNHLSASESILRSAWAKVLRVDPEQIDVDDNFFRRGGDSVLAMKLVSSLRAQGYSLSVADIFRHMRLSDAARVMKVDERSTEKINSYQPFSMLRLPDVQQFLANIVRPQLGDQHWPIRDVLPVTDSQDMDIRATIQPPRTSIQYTMLYFDNSVDRERLFRSCSDLVKTHEILRTVFISHESSFLQVVLNELEIPVRAHKTDKQLDQYVASLFREDIESNFQLGCPFLRLFYVEGNNGESCLVIGLSHAQYDGVSLPRLLQDLDALYTGTQLATFSPFSLYMAQTSEEAIQNKAAAYWRNLLSSSSLSTLDGPSSDPTDKAIFHTRPVNIHPLKEITTANLLTAAWAMVLARRLQTPDVTFGSVTSGRTLDIPNAENFMGPCYQLTPVRVPFHPDWTASDLLNFVQTQSAESAAHDFLGFEKIAKLAGWASGRQGFDSIVHHQDWEDFDMMPFGGGSCRVDIANPHGDAAYPVKAVSFVKEGEIHVGVVCSERDVMFVDEVLGELAAAVVELAGQSTEVLLDSKLFSGQ

SEQ ID NO: 7 - DNA, 813 bp, Aspergillus fumigatus
atgtcaacagaagcacatccaacagtacaaggatgcctcgtctcgtttccaacacctcacattctcctcctcacactcaaccgcccagagaaaaggaattgcatatccctcgccacgagcgccgagatacagcgcctatggacatggttcgacacacaacctgctctatatgtggccattattaccggcacgggcgagtccttttgtgcgggcgctgacctcaaggaatggaacgacctcaacgcgcgtggtatcaccaacgaaatgacagcccccgggttagccggcctccctcgccgacgaggcagcaaaccaatcattgctgccgtcaacggatactgtctcgggggcggcttcgagatggtagccaactgcgatatcgtggtggccagcgagaacgccacctttggtctccccgaagtgcaacgaggaatcgccgccgtcgcaggctcgctccctcggctggtccgcgtgctgggaaaacagcgagcagccgagatcgctctgagcgggttgagcttttctgcgtcccagctcgagcggtgggggctggtgaaccgcgtggtggagcatgaccagttattggcgacagcggtggagattgcaaccgctatctctaggaatagtccggacagtgtgcgtgtgacgatggaagggctgcattacgggtgggaaatggccagtgtagaagaggctagcagtgcactggttgaccagtggtatgcaaagctcatggctggagaaaatttccacgagggagtacgagcatttgtggagaagcgaaagccacagtggaggccgtctaagttgtga

SEQ ID NO: 8 - protein, 270 aa, Aspergillus fumigatus
MSTEAHPTVQGCLVSFPTPHILLLTLNRPEKRNCISLATSAEIQRLWTWFDTQPALYVAIITGTGESFCAGADLKEWNDLNARGITNEMTAPGLAGLPRRRGSKPIIAAVNGYCLGGGFEMVANCDIVVASENATFGLPEVQRGIAAVAGSLPRLVRVLGKQRAAEIALSGLSFSASQLERWGLVNRVVEHDQLLATAVEIATAISRNSPDSVRVTMEGLHYGWEMASVEEASSALVDQWYAKLMAGENFHEGVRAFVEKRKPQWRPSKL

SEQ ID NO: 9 - DNA, 1389 bp, Aspergillus fumigatus
atggcgactcaaagcagcaccgagcttcctcaaatcaatatgaccactgccgagccaacctcggccaataagcgaacatatccctgggtcaaacttcctcatccctatctcaccagttatgcgatccatgttgtttctgaatctacaccccgggtgcttcaattgagactccatgacgatcaaaagggacaggctctcccagagcctctgcactcggcctcgttgacctacaccgacattgcctttgacgaggccgcccaggaaatcccagataacgacaatagcccatgggcgcgcagtcgcagagctcctggaacctccttccactggactggccaggagccaccgactctgggacagatctggaacgtcatccacgccctcttcctgacatatccccaacacgagattgtccgcttggatctgaacgggtctggcaaggatattattcgggaggaatgcctgcgcactgggctggctgttccctttccttcccctcgagttcccttcggcactgagaaccggccctccgaaaccgacacactcatcctactacgctctgccttctggcaaggagccggctcaccggttggaccacgtccgatatgggccgtcgaccacgggattcacggcctgcttcgacgatcgggaagctcctatccccctctcgctcaaaactacgagttctccatgaagttccccagcgagagaatctacacccggcaccctatccgcccgcagaaaccagccccaggatcactagtctacagccgctacatcccacacctggaccagcatttctcactcatggtggtcgactggcaaaacgaagagcacctgcagctcttccacaagtggcagaacgacccgcgcgtcgccaagggatggaacgagaccggcgacctggagcaccaccgcaattacctgcgccagctccacgaagacaagcatgtcctgtgtttattcggacggttcgacgactttccattctcctatttcgaagtctattgggccaaggaggaccactacggagcacactacgatgccggagactatgaccgtgggcggcattcgctcgtcggcgagtccagcgtccgcggcgcataccgagtcaacgcgtggtggtcgagcctgatccattatattttcctggatgagccgcggacgatgtgcgttgttggggaacccaaggccacgaataccactgttctgtcgtatgagaatgcgcatggcctgacggtgcagaaatacgtggatctgggccataagcgatcggtccatgtgtattgcagccgggagaaatggtttcagctgtgtccccttttctgggatgggagggaaagaccactggaaagttcggatcggatggcgtgggatgcaaagctgtaa

SEQ ID NO: 10 - protein, 462 aa, Aspergillus fumigatus [sequence omitted]

SEQ ID NO: 11 - DNA, 20000 bp, Aspergillus fumigatus
ggtcgcaacattgacctgcctgaccctcaaaccatcaacggcacctgtgcaaacttccttcccatgcggtcgaaagtggacccgtcaatgcctgtgtcggaattccttcgtcaaacccaggatgaattctggcagtacaccgaaaacagtacggtcggcatggacgagatccacgaagcctgtgaaacgacgcgtgaaggattctcgaacaaaactctcttcctcttccagccatttgaaccggccccggcgacagcaaagcaatatgagaagtggattgtgatggccaagtcccaggtgacgatgccgcagccatatgcggtcgtatttgaggttgtcaagacagcggatgtcaacgagtataagctcaagtttgcctttgacaaacgtgtttatgagaaagaacaagtacagggggaggcacaggtgattgagaagttgttggccaaggtcatggagaatgctgaggcgtctgtgggtgatgttctgggatcgtttcgctcttgattcatctaaagattgtatatagttaaaagttgaagaatctgttttgactgagaaagagtaccttgccttgatttgatcccatatctttgtcttcatcgattccagccaggagtgagttgtcgatggagtggttgggttagaggcgcagctgagaaaacatccatcgcttgctggaatcaccacaaacacattaaccatccaacgcatcagataacgctcccacttcgtcgagctcagacttaaagaccaccaggtccacgtcttggctccaaatctggaatcgtcccagcgagcgctcatccttctgactagtcgatgcaacacttaccaacaaggccaagaaagacacttgcacatcgtcttttgtacttttgcccttaggctgtatttaatgcctgatgctacccaggccgctctgcgccgataaacagcttcctcgagcaccacccaccgtgacggaagcagttacgatcatccacacttttgcagatggacattatggatatgtgtcctgagtattcaaagtattatacaccccggatcctcacttattacaattacaaagccgcgacagccaaggcagcagcagccacagccccaaccacgttcaactcgagaattcccgcaccgttccctgtctcagtagcaccagcagaaccagtagtcgtcgaagccacactagacgagcttcctgctttgacactcgtcttcatcgtctccgtcgcactggcagtcgtactcggcgcagcggtaatccgcagactactcgcctcgctcgacattgtggtcatcgcgtcccctgtagtcgacgcgctcgccgtcgcagcaagcgtaggtgtagacgcagccgcaccaggcccatacccagggagatagctcggctcgtaggtcgcggtaccggtgtacagctccgggcaagcgggattatcatgcttgggcggggcgcggccgccgccacagtcggggatgtcgcagatttcgcctgtgtcggggacgtaccagatcatgctggcttcgttccattggttgcgggtgagggaggagatacagcctctcaggtcggtgcgggcgtagacggtggaggcgagagttgtcgctagaaggaggaagaatctcattttgctggagagggttcgtagaagaggggtatcagatgcgaagtaaggagcagtacgttaatgaatgatattcaaaagagtgactgatttttgaaaggaaaggagtggtcggagacaagagtggaggtaatgttgacgatgtgagggactgaaaagtcttataagtctggcttgggcaatctagacctggcccaatggaatctgggtttaggcctaatgagcttagactaaagacggctgactcgagacgaggctgcaactacgggattgagatgcattggatccaattaagagcggcgagactgtgtccaaaatgatctaatggtttgacacatcgtgatttacggctcatatggcaagtttttccaagttctgaaatcccatggaaagatgcagtgtgagcctgattcagggaccatcggcaattcgatgtcggcatctccgactctagtccactttgggtccccttccgaaattgcgagtctcctaattttagttcaaggttcagcctcatgtttggagtggagctcaggacctgttgacttgaccagtgtagttgccgcatgagaaaaaagtggagtgcaaccggcaggaagtaagggtgaaagtaaaacgtttagagatgtggaggctaaggacaaaaggccgaccaaaaggggggcaaaaaaacaaaatggcaaaagaaatctccccgacagggagtcgaaccctggtctcccgcgctcgttctcattagagtgacaagcggaaatcatgaccgttagaccatcgaggatttcttgctgattttttgtgatttcaatatataagagtatatcaggattactctgcacacaagacctaccactccattgatccaaggaatatgtaatgtttgttccaatttctcgtattttattataggaatctccagtcccatgttttcgttagctttctttgactcactgcgctgctcacatgtagcatctcactgatctcatgactatatatcttgccctgcgggtaaacttcggccttgacaatgtggattgcatctatatcttcgttccttactggtgaacattggcatggcattgcaacaatgtcattcatgatatctctaggtctgacatgcctctacggaatgtagggccgacttccccgggatgaatggatagcttcatatgctcgtattctcgcaccaatcgaaggggtcctattggtgcatgtatccctcttcttactgtagatctaactgacaagtacagcacagatgagaagtggtcatcagacaagcttatctcaatcgatacaaacagggacagcgacattggtatataaaccccgacaactcagcgcaacaaacattcctcacactgcagactcaagctcttacaatcacaatgatgcccagaacggagaaagacagccagatcatcgaaaaggccaaaagccttcagggtgtctgctggggcgaggaatatgagaagatgatatcagggatactgtaaggacattgctcagaacaccgaaccactgctaagccgctgtacagatatagtccgctcgcgtcggagctcatagagggtcgcaccagggcgcgcttcctaatgtcggaattcaatggtcctcctaaacccgatgtgccgttctctgagaccgttgcgcagagggaggtgacattaaggaagttgtttgggcgggtaggaaggggcgtctatatcgagcctcctctctttgtggactatggctgcaacatcagtgttggagacggtttttatgcaaacttcaagtaagccgtgcttctaacaactgctcccggatgaggctgatagagcagtttgactgtcctggactgtggactggtcaccatcggaaacaacgtggagattggcccgaatgtaaacatcatcacgggcgagcacgagacgaagatcgaagcccgtcgcaaacatcgaggaacggagttcaccagggaagtcattatcggtgatgattgctggattggagcgaatgtgactatcttggctggggtgaccattggatcagggtgctccatcggtgctggctctgtggtgaagagagatatcccgccgtatagtattgcagtgggaagtcctgctcgagttatcaggtcggcagtctgaaaatgccgctatgccccctgtgcagacattgtcttgttccaatcatattacttgcctcggggtacatctgtgctgagccgacaatgtcattcctaaatccatgtctcaaagcatcctctttcgccacttacactatgagtgactcaactgtctcttatttttgctccttttctatattacaacacttaacctaagcttttactaaagccgaaagatatcctagtaccttgttgaccagaaagttccgaggctcagcttgatcatcattactcaatgcatgagacgcattcggtatgataccgctgttttcgtcccatatcgcggcttccccattatgatctgtggcattcctccacctagacagtaacgtgtccttgtcaacccaggccggaaccgactggtccgccccagaaaacaacaccatcagtttccccctcaggagaccctgctccctgatcatcccgaacgtcttccccaactgctcatcgctcagatccgaactaaatagatcatcctcactcggcgactctggactctcaggactcgtcagactcagaaaccgccgcgcactcagcggcgtatgagccgggtataccagcgaagtcatcgccaacggcagcaggacatcagtgccagcgtccgcgtcgcgagcagcctccctcgccatgctagtcaacttctcaaacacaggccggatctccgccggcgtcctatctcccaacccctcggcaaggacccactggatcgcttcccggtccgacacgggcgcctgcatgatcgccccgtcgagcggcattcgctcaacatgctccaggtaggggtcgaaggccggggtgtgtgtgtgcggatttggtcgggacaggtagtgaagcacgcactggctacctgttgagtggcccataaggacgatcttcccgctgctagcgctgccgccgaatttttcggttttgtactccttgatgtacttcaggcactgtgcgatttcgttggtgtctcggtcgaggtgccccagcccccaggattggtacgaggaggtcagggtcagggtgaagagggaccattctgttggttggagggcgtgagctagatccgccaggtaggatgttgttgctaggccatcgccgagaccgccaacgaagaggagactgtgtggtttgcgggtggtggttgtggtgtattcaaatgtgacgagggtttctgtctaaattgcagatcggttaggtactctgtctgttgcactgtacgaggggaggaaacgtacgtagtggtgtagaatgcctgggagaccgcctttgggccagaatttcgagtacatattcaatgtgtatagagcagcaattggtcgttttgattgtggttgaagctgggacggaaaaagaaaatctgcagggaatagcagaaatcgcatccagaaatgcattggtgccatgcccctcgtccaatctgataaatggccgagatcggcactgcaggttcaagcttgtctcggtgcctcatgtgctgctgcacttggatggggttggcactaagcggaaagcccgttatcaatcttacatacgttacatagtgggacctttccatacttgtacagatacttctctacaatacattggcttggcctaatacctgtcacgttcctcataagagacatgacaagtggagatggctaggaaaaatcttacgtacacctggctcgctaacaggagccagaatgactagctagatctgattagatttcttgggacaagaccaagcggatctgattagatgcagttcggctacaacccctgcttcgggttagaccgggtaaacgccagtttcgcacaccgaaaacttctccgttctcacacaaatcgagtgacactgtttgttgaggaattagacagtggcctgatggaaatatttcatattggatatacaaaggaaatgcatccaggggccatggttcatgaacgcgactctttggtcttggagacttggacgccaaataggtgcggtagacaatagagacgagcgattccatcattacagcccaagaacagcagaacgcacaggcacatgcacagtcatgagcttctcaaccttggcctaggcttgtgactaaagaaagttcgatagacgagtactcctacatgtcacaccatgtttaaacacgttgtattgtaccataagtttggcgggtaatcttaccatctgtaatgagaccggacattacacgccacggttatctgatccaactatggagcactctacgtattcaaccaaggaggtcaggccctaccgggctgccagtggcagaaaccagtgtaatgtgtgtatctccatcgggcaattccggtgaatggcccactggcttcggatcagataactttgcactatgactgaaccagagaatgtgcctgtggctttcctagaatactactccgtattctgtatgaaaacgcgaatttatgtgtcatgggaaagcctctctctgtggggaaaacagttcgcctaaaaatgcccaactaggcgaggattctgcaaatagatggaaagatgggagtaacattctgtcacaagcaccaggacgggtgatctttacagagagactgggtccgagaccgaatgagatcaagcgagcttggttagctggcagggatgacagtcatggctccataggaatggctgtggactcggctaactatctgattagattgataatatgtctgaattatgacaaggtgcacatgagcctggaccagtcattggtggcgtttgagtcttatcacactctagtccctacactgaagtatcccgtaccctcctcaggttacactcatctctttttacattgaaagaaattaaaattgagcctagtaattacagctttgcatcccacgccatccgatccgaactttccagtggtctttccctcccatcccagaaaaggggacacagctgaaaccatttctcccggctgcaatacacatggaccgatcgcttatggcccagatccacgtatttctgcaccgtcaggccatgcgcattctcatacgacagaacagtggtattcgtggccttgggttccccaacaacgcacatcgtccgcggctcatccaggaaaatataatggatcaggctcgaccaccacgcgttgactcggtatgcgccgcggacgctggactcgccgacgagcgaatgccgcccacggtcatagtctccggcatcgtagtgtgctccgtagtggtcctccttggcccaatagacttcgaaataggagaatggaaagtcgtcgaaccgtccgaataaacacaggacatgcttgtcttcgtggagctggcgcaggtaattgcggtggtgctccaggtcgccggtctcgttccatcccttggcgacgcgcgggtcgttctgccacttgtggaagagctgcaggtgctcttcgttttgccagtcgaccaccatgagtgagaaatgctggtccaggtgtgggatgtagcggctgtagactagtgatcctggggctggtttctgcgggcggatagggtgccgggtgtagattctctcgctggggaacttcatggagaactcgtagttttgagcgagagggggataggagcttcccgatcgtcgaagcaggccgtgaatcccgtggtcgacggcccatatcggacgtggtccaaccggtgagccggctccttgccagaaggcagagcgtagtaggatgagtgtgtcggtttcggagggccggttctcagtgccgaagggaactcgaggggaaggaaagggaacagccagcccagtgcgcaggcattcctcccgaataatatccttgccagacccgttcagatccaagcggacaatctcgtgttggggatatgtcaggaagagggcgtggatgacgttccagatctgtcccagagtcggtggctcctggccagtccagtggaaggaggttccaggagctctgcgactgcgcgcccatgggctattgtcgttatctgggatttcctgggcggcctcgtcaaaggcaatgtcggtgtaggtcaacgaggccgagtgcagaggctctgggagagcctgtcccttttgatcgtcatggagtctcaattgaagcacccggggtgtagattcagaaacaacatggatcgcataactggtgagatagggatgaggaagtttgacccagggatatgttcgcttattggccgaggttggctcggcagtggtcatattgatttgaggaagctcggtgctgctttgagtcgccatcgcgcaatgagtttgcgagtaagatgattcttctgcaggcagcaaagacgtaggagaagtacatatatgtaagacatggccgaggaatcttcagaagccgcggtaggaagtggagacaggaggtctagacgataccgactatccggtataatcctctaaatccggccccaaatatgacttaaattacttgatccctatggacaagagtataagaacactattcctcattgagaatcgttaacagactgcttaaggcgccataaatccagtctcagactgtatactgtatctccgacctacagacaacaacaatgtcaacagaagcacatccaacagtacaaggatgcctcgtctcgtttccaacacctcacattctcctcctcacactcaaccgcccagagaaaaggaattgcatatccctcgccacgagcgccgagatacagcgcctatggacatggttcgacacacaacctgctctatatgtggccattattaccggcacgggcgagtccttttgtgcgggcgctgacctcaagggtacactcctcttatctctattcgatggaaacatctctattctaaggaaaacaactgacaatacatcccatagaatggaacgacctcaacgcgcgtggtatcaccaacgaaatgacagcccccgggttagccggcctccctcgccgacgaggcagcaaaccaatcattgctgccgtcaacggatactgtctcgggggcggcttcgagatggtagccaactgcgatatcgtggtggccagcgagaacgccacctttggtctccccgaagtgcaacgaggaatcgccgccgtcgcaggctcgctccctcggctggtccgcgtgctgggaaaacagcgagcagccgagatcgctctgagcgggttgagcttttctgcgtcccagctcgagcggtgggggctggtgaaccgcgtggtggagcatgaccagttattggcgacagcggtggagattgcaaccgctatctctaggaatagtccggacagtgtgcgtgtgacgatggaagggctgcattacgggtgggaaatggccagtgtagaagaggctagcagtgcactggttgaccagtggtatgcaaagctcatggctggagaaaatttccacgagggagtacgagcatttgtggagaagcgaaagccacagtggaggccgtctaagttgtgaaactgctaacaggtggttgtcaattttgccaggatgaaggaatacagctaagtgcgccattttatacagttcttatctagggaatattgattgtgaacagagggtactgacgttatttcgtacgagagtgtaataccatacgcgacgtgtattgatgtactcagtaatactgtatgtatgcttacccactctactggtaatatcactaaccaaagtcagatagtaaaagaactgcgcaagccaggcttgttagaatggctattaactggttgagaatgctttagtgggatcagataacagttacccctccatctcgcaaggctaatatgatgctcgagccgaatattcagccccactgggttgaagaggcccaaactgtaggcggcattgtaattttgtcttcagtgaactaggatgatattacctggtttgaatgggtcctgtccacttgtttgcgcaatgggacacatgctcgctgcccctccatgaaagagacgtgggtattcatctggtcatggtcacggcgatctcggtagatgtgtagactgctttacatagtgttttcggccgctcatcggtggaataatgccaacaacctaggctctccattatatagttaacgcatgccaaatccttttagtcatccatcgcacacatctcccgctcccaggaataacgctccaggatcaagactagaacagccggacaatgggttcaatacagcaagatgacgttcacaatcagatagaccactgcaatcagtcagatgacttaccggcggcccggttgaattgcaatgacgtcgaattattcgaggtggctggcctcgcatgcgacgagacctcgagtcccacaggcatgagagacgaaatggtacttttatcgtggctcattgcgctcctgcggacacgagaaggcggccagattcggtacgaatgggcatataggtatccagaagaggagccggtacctagatgtctggctatgaatgaagtagtggcggggttacaaagtagtgtcaaggaaactgcagcagctgtgtctcggcatatttccgcagatgtatcgagtccgccggcgccagcatctctgcttctgagcactagctccctttctcagacatcagacgaagcaaaagatgaagtaagtcaatgcgtgggttttgaagacgtgagcctagctgactgcgggaacaaaggggttacttcacctagaaatcgctttcgagaatgggctgtgcaagattcgtccgacatggcactccgagaatatgttgccgtttacagtgacgcgatacgccaggaccctcatcgacacggttaggctgtgcatttccaattgcgatgccgccatccaagactgccttcggcctaccgcatacgatctcgacgagatatggcgctggaaccacaacctgcctcccacgtacaatttctgcatgcacgaaataatctccgaccaggctcagaagttccccgacaaggaagcaatcgcatcctgggacggcagcctgacttacagacagattgatcagtattcatcattcgtcgcgcgctcacttataggcatgggagttgggctgcatgatgtcttaccagtctgctttgagaaatcccgatggacgattgtcgcggttctggccgtaatgaaagctggcgcgacgtttgtactaatggacccgacgcttccacttgcacgattgcagaacatggcccagcaggtcggcgcgaagatgatggtttcatctcggggccagtacaacctggcgacggagatcatacccaatgcaaacgtgctcgtcgtggaggaaaacacgttcagcagtctgtccgccgaacagaacggcgagcccctgccaacggttccctcctcggccctgatgtacatgatctttacatccggcagcacaggtactccgaaaggagtcaaaatctcccacgaaacctacaccagtagcgccattccccgggccaatgcggtaggttacacggaggattcccgcgttctcgacttcgcctcctacgccttcgacgtcagcatcgacagtatgctgctgaccctgggcaacggcggctgtctgtgcattccgtccgacgaagaccgcctgaacgacatcaatggcgtgatacggcgaatgaaagtcaactatgcgggcctgacgccttcggtagcccgtattctagacgcagatgtaatatcctcgctcagtggtcttggcctcggaggagaggccgtctcagcgagagatgtcaatctctggggtcaggacaccaggattattatcggctacgggccctgcgaatgcacgattggatgcacagtgaatagtagcgcggcaacgggaagagactatatctccattggccctggtaatggggcagtaatctggatagtcgatcctaacgaccatgaatctctcgtgcctctcggcgcggtaggagagctgctggtagaaggtcccatagtagggcagggctacttgaatgatccggagaaaactgcagcagcgttcattgaagacccctcatggttggttgcgggccacgaaggttaccccggtcgtcggggccgtttgtacaagaccggtgatctcggtcgatatgacccagatgggtcaggcggtatcgtgttcgtggggcgcaaggatacccaggttaaattgcgaggacaacgggtagagctcggggagatcgaaagccaattgagagccagactgccctcggagacgactgtgatcgccgaggtgattgtaccccaaggatctgggggacaaccgacgctggtggcatttgttgctgcccagacgaccaagggacatgatcataccggactcgaggcggcagagcttcccgatgagcttcgaagggcgctgtcagaagccgatgcggagctggcaaaggtattgcctcgatacatggtgccaacagcgtatattcctgtcaaccacatccctacgctgatttcaggcaaaacagaccgcaagagactgaggcagttcggcgcaaccgttgacctgcgccagctggaccaggatgctacgaacaccgcagctcgggagctgagtgatctggagcgtcgcttgcgacaggcatggagccagacgttgaagctccaagcgtgttctattcgtctacaggacaacttcttcgctcttggtggagactccctcacagccatgaagctggtctctgtatgtcggtcccagggtctcgacctcagtgtcaccagcatgtttagcaatcctaccctgtcagccatggccagcgttgtccgtatttgcgacgtcgacgtgcaaaggacagtcccggcattctcgatgatcacatcagatatgaacagtgcctgtgtagaggcagcagagccctgcggagttggcccagcagatatcgaggacatctacccctgtacgccaacacaggagtctcttttcactttctcgctcaagtcagtcaagccctacgtggcacagagagtcctatgtatcccgtctcatatcgacctcaacgcatggaggaaggcatgggaggatgtggtcgccgcactccccatcctacggacacgagtagctcagctgcaagagcctggccttcagcaagttgtactcaagaacagcatttcctggacgcaagcctcagatctagcggaatacctagagaacgaccgaacccaaaagatgaatctcggagagagtctggcccgttacgccatcgtcgaagactcagccgacggcaagcgatacatggtttggactattcaccatgtgttgtacgatgggtggtcagagccgattatcctcaagcaagtcagcgacgccctgcaagggcagccagtcgaggtaaaggcgcagatgcgagactttgtcagattcgtgcgagactcggatgacgctgcagtgcaggaattctggcggcgcgagctgaaaggtgccgtcgggccccaattcccacgtctaccctcaagagacttcatgcctaccccggacgccctagtcgagcgtcaagtctctcttgatactagctccggatcaccgtttaccatggcgacattgattcgcggcgcctgggcgctcgtcgcgtcccaatatactggaagcgacgatatcgtgttcggcgagaccctcacaggtcgcgacatcccactacctggagtcgaaagcattgttggaccattaattgcgacggtccccatccgcgtgcgcattctccggggaagtactgtggagtcctacctccaagccgtgcagcagagtgttttggcccgtaccccgtaccagcatttgggtatgcagaacatccgcaaggttagccaagatgcgcaacacgcgtgcgagactggtacgggtctggtcattcagcctgaaccagagtatgtcggcagcgaactgggcgttgagagaggggacgtggttcttgaagcgctgcatttcaacccgtatccgctgatgctggcttgcgggattcgcaagggtggtttccgggtttgtgcaagctttgacagcagcttgattgagaccaggcagatggaacgaatgcttgcccagcttgagaccgcgtgctggcagttgagtcaaggtctttcccggaaggtcgatgagatctcgtgtctccccgaagccgagttgaaccagatctggcagtggaatcggagtccacctttgtctctggacgaaacaaccagtcggttgcgtgcgaatgcaagcacaaagcctggttcaagctatcctcccgcggttgttccttgggtttgtagtccacgaaactcatctctcctctcgccaatcggctgtgttggcgagctctggcttgaaggggcccttctttcgggcgacacagttgattcacctgcctggcttgtggctggaagctctacctgtgctggccgaactggcaaagtgcaagcaaccggcgacatggttcagttgcgagaagacggcagtttagtgtttgttggtcggaaggagaatgttgttcctgttcaagggcatgcagtggacatcacagagattgaaaggcacctcgcagaacatctgccgccaacaatccgtgccgctgctactgtagtgcggtcttcatccgaccaggagctggttatgttcattgagcaaccggctgcggaggaagcctgtattgagctcctatcggagaagcgcgaaatagtgtgcgacgcgccagacaaagccttccagaccacaatttgtgcgacaatcccgggcagtcttgctgcagtgttgaagaagttggacaaatacatgcgagactcgcttccgtcatatatggctccgtccgcttacatcgtggtagagaaactgcctaatacgatggacgatatcgaccacaatttgctcaatcagatcgcctctcaggtcactccacaaattctcaacgagcttcgcgatgggttgagcaacgcttggaccaaggctacggctccaaaccatctatcagcttcagaaagcattctgcggtctgcatgggccaaagtgcttcgtgtcgatccggagcagatcgacgtggacgacaatttcttccggcgcggtggtgattccgtacttgcaatgaagcttgtgtcgagtctccgagcgcaaggctacagtttgtccgtggctgatatcttccggcacatgcgtctcagcgacgctgccagagtgatgaaggtggatgaacgctcaacagagaagatcaactcatatcagccgttttcgatgctccgtctcccagacgttcagcagttccttgccaacattgtccgaccacagctaggggaccaacactggcccattcgagacgtgttgccggtgaccgactcccaggatatggatatccgtgcaaccattcagccgccgaggacgtcaatccagtatacaatgctgtactttgacaacagcgttgacagagagcggcttttccgttcgtgcagcgacctggttaagacgcacgagattctccgtaccgtcttcatatcacacgaatccagcttcttgcaggttgtcttgaatgagttggaaattcctgtccgcgcacacaagactgacaagcagctcgatcaatacgtcgccagcctcttcagggaggacattgaatccaacttccagttaggatgtccattcctccgacttttctacgtcgagggaaacaacggcgaatcctgcctcgtcatcggcttatcccacgcccaatacgatggcgtctccctgcccagactcctccaagacctcgacgccctatacaccggcacccagctcgccaccttctccccattctccttgtacatggcccagaccagtgaagaggcaattcaaaacaaggcagcggcctactggcgcaacctcctctccagctcctcactctcgaccctcgacggaccctcctctgatcccaccgacaaagcaatcttccacactcgcccggtcaacatccatcccctcaaggagatcaccaccgcaaacctcctcacagcagcctgggcgatggtcctcgctcgccgtctccaaaccccagacgtcaccttcggcagcgtcacctccggccgcaccctcgacattcccaacgccgaaaacttcatgggcccctgctaccagctcacccccgtccgggtccccttccatcccgactggaccgcaagcgacctgctgaacttcgtccagacacagtctgccgagtctgcggcgcatgacttcctcggttttgagaagatcgctaagctcgcggggtgggcctcaggaaggcaaggctttgattcgatcgtgcatcatcaggactgggaggatttcgatatgatgccgtttggcggagggtcttgtcgggtggacattgcgaatccgcatggggatgcggcgtatccggtcaaggctgtttcgtttgtgaaggagggagagatacatgttggtgtggtctgtagtgagagagatgtgatgtttgtagacgaggttctcggggaattggctgctgcggttgtggagttggctggtcagagtacagaggtgttgctggatagcaagttgtttagtggacagtgacgatctgggggtggatagtgtatggttgattgatttgtgattatatgtcttcattgcggagcacttatcaaacgatggtatatttcaatgatcaagtctatgtaaggattaaaatatgtatcaataactacgccacagtctgatgcataacattggccctataactctccgacctctgcagcaactcctcatgcgtcccccgatccacacactttcccccctcgatcaagaaaatcacatccgccttacgaatggtatgcaaccggtgtgcaatggctatgaccgtgatccccttcgcggccctctccaacccatcctgcaacagcttctccgactccgcatccaacgcactcgtcgactcatcgaggatcaatagtttcggtttccgaaccagcgccctggcaatcgacagccgctgcttctgacccccggagaactgactcccgttcggcccacagagcgtctggtacccgtccggcagggactcaatcacatcgtggatattcgcgagcttgcacgcctcaacgatctcgtccatcgttgcttcgtgatctggtcgcgccccaagaccgacgttgaacgcgatgctgccctcgaagagcacgctctcctggggaactagcgccatggtgtctcggaaggagacgtcacggtgtttggtgatgtctacgccgtcgacaaggatagcaccggactcgggggtgtagagccgttctacgagggagatgatggtcgatttacctgcgccgctgggtcccactagcgcgcagaactggcctggccggatgtcgatgctcaggccgttgaggactttgatgtctggtcgggcggggtatgcgaaatggacatcgcggagctgcacgcttgaggcttcgtggttggaggctatgggctttggctcggctgttgcctcggggtcgttcgaatcatttgtgggagggctgcgcacgtgtccttgcatcttgtccgagccgatttcgatgagacttaggatccgggccattgcggcgcgggcgctggagagctccggtgcaagagcgaacatttgactccacaggagtgcactgacaagcagggagaagacgacaatcaggaactgggtttgcgtgtagttgcctgcaatgatctgcttcgcaccccaccagtatgccagcgcgttcaccaggttgccgaggaagtaggtcattgcctgccacaggctggcgtgcatagtgaccttgaatgtttctttgcgcggtcccttgagcgaccgtcgatacgttcggagcgtctcctcttcgagggagagggatgcgatggtcttgatggatgtgatggcttccacgccgatatcaacagactttgtatacgcgttttcgtgacgctcctcaaacttcccgagcacatggagctccatcagtccagctccaagcagaagtggcaccagcgccaggcagactaatgcgatcctccaggcgatgatgtgagtcagaatgattgccgcgatgaggttgacactaatggaaaacaacgtgccaatcaccgaaccgctcaagccagccaaggcattgccatccctggtgatataggacaaaagcaaggctggtgtgcggccctcggactgatgccattgcaagtcctgctcgaagagtgaccggaatgacaagacccgaaccgcgtaaacgatcttctccgacacccagccaaacccagaccagctgaccacgttggcgaagaattcaataatagcgaggataaagaacatcaacccgaagaagtttcctcttgaccggatgctgtctgcgctatggcagggattcagactgcccacggtgtttccgaagatgacagcctctccggagaaggcgccgccaaccacacttgagccaaggagtgcgaggaaaataaacagtagatgtggtcgtagtgctggcgcgtagcctttggccagcgcccagagggactttttcggagtctccggttcttctgcttcctctgccgcgtcggctacaggagctgcggtggtcgagactggaatcttctcggcgtcgccaaagtcgtcgaggagaatggcacttttctcaatgtcagcctcgcttgaagatgtctggtcagacttgctggcggcgtcgatgttgattgttgtctcatccttgcttgcaagagtctgcagtttgacaagctcagcgtaggtgccattcttagccatgagggtcgcatgattgccttgttccacgatatggcccttgtgcatgacaatgatgttgtccgcactggtaattgtcgataagcgatgggcgatcgtgatgatagtcctgccagaggcaatgttagagattgcccgctggatgcgctgttcactccgtgaatccagagacgccgtagcttcatctagtatgagcactgcaggatccttcaccagagccctagcaagcgcaacacgctgcttctggccaccgctgatcagccggccactggagcccacaactgtaccgtatccgtgctgcaggacactgatgaagccgtccgcatccgcaagcgacgcagctttggtgaccaaaccgactatttcagcgacatttggaccccgcgcctctgctgcagcggtcaaatccttgccgcccctgacttcactggcgaggtcggccaggtcagtgctcaacagggttgccttcaggtgtgcatggctcggatggttcgagttgatcagcccatgagcaatgttctctagaagcgaccggtccaggagagagggctcctgctgaactaggctcataaagcttctcaagtcccgagggttgacatcacgcaggtcgtggccgtcgaaaagaacttggccctgcgtgggatcgtagagtcgtgtcaccaagcccgcaatggtcgacttgccactacccgatagaccgacaatggcagtatgcttgttggccgggatgctaagactgatgttgtcgagcacggtgatctcagggcgcgagggatacgtgaaggaaacgtccttgaattcaaaaccgccctccgcctgggtaagccgaagtccggaaccgctcgtactgtcgatcagcggctcgcggtcaatatcctcacgcaacttctggaacgaggcgacggctgcgacgaagaggtggaggaagggcgcgacttgactaaggagcaaggtcgctgttcgcgttagagtggctcactacccaaggttgaatcgaactcaccttcaacaagaatgaaaatcaccgtaaacgtagcccctaccgtggcaccgtgagtattgccactcacagaatcggctatgcttcggctcccctgccagaacgccagaccattggcagaatatgcaatgaagtataacactccagattggatgccaaccgccgtcgccttcttcaacccttccgtctctgcagccttgagtgccttggaaaatttctcctccagccgcgcattggccccaaaggcttgcaccacgacggtgttggaaagtgcctcggaagcgatggacgcggcactcgcagcgtagtccgacatgcgaccagtgtacttctcgatatagtggcttccgacgaacgacatgagaaagtatgccgggattaacgagacgagcattccggcaagcttggcatctttgataaaggcgacgatgtatgcggtgatgaaaaacgacagactcgacaaacatatccccactttctccgacgtgccggatcgaatagcctgtatgtcgccattaagtcgcgatgcaatctcaccggggggtagcttgtcgaagtaggatggttcttgtcggaggacgttctgtagataagtttcccg

SEQ ID NO: 12 - DNA, 1125 bp, Aspergillus fumigatus
atgtactcgaaattctggcccaaaggcggtctcccaggcattctacaccactacgtacgtttcctcccctcgtacagtgcaacagacagagtacctaaccgatctgcaatttagacagaaaccctcgtcacatttgaatacaccacaaccaccacccgcaaaccacacagtctcctcttcgttggcggtctcggcgatggcctagcaacaacatcctacctggcggatctagctcacgccctccaaccaacagaatggtccctcttcaccctgaccctgacctcctcgtaccaatcctgggggctggggcacctcgaccgagacaccaacgaaatcgcacagtgcctgaagtacatcaaggagtacaaaaccgaaaaattcggcggcagcgctagcagcgggaagatcgtccttatgggccactcaacaggtagccagtgcgtgcttcactacctgtcccgaccaaatccgcacacacacaccccggccttcgacccctacctggagcatgttgagcgaatgccgctcgacggggcgatcatgcaggcgcccgtgtcggaccgggaagcgatccagtgggtccttgccgaggggttgggagataggacgccggcggagatccggcctgtgtttgagaagttgactagcatggcgagggaggctgctcgcgacgcggacgctggcactgatgtcctgctgccgttggcgatgacttcgctggtatacccggctcatacgccgctgagtgcgcggcggtttctgagtctgacgagtcctgagagtccagagtcgccgagtgaggatgatctatttagttcggatctgagcgatgagcagttggggaagacgttcgggatgatcagggagcagggtctcctgagggggaaactgatggtgttgttttctggggcggaccagtcggttccggcctgggttgacaaggacacgttactgtctaggtggaggaatgccacagatcataatggggaagccgcgatatgggacgaaaacagcggtatcataccgaatgcgtctcatgcattgagtaatgatgatcaagctgagcctcggaactttctggtcaacaaggtactaggatatctttcggctttagtaaaagcttag

SEQ ID NO: 13 - DNA, 6307 bp, Aspergillus fumigatus
atgggttcaatacagcaagatgacgttcacaatcagatagaccactgcaatcagtcagatgacttaccggcggcccggttgaattgcaatgacgtcgaattattcgaggtggctggcctcgcatgcgacgagacctcgagtcccacaggcatgagagacgaaatggtacttttatcgtggctcattgcgctcctgcggacacgagaaggcggccagattcggtacgaatgggcatataggtatccagaagaggagccggtacctagatgtctggctatgaatgaagtagtggcggggttacaaagtagtgtcaaggaaactgcagcagctgtgtctcggcatatttccgcagatgtatcgagtccgccggcgccagcatctctgcttctgagcactagctccctttctcagacatcagacgaagcaaaagatgaagtaagtcaatgcgtgggttttgaagacgtgagcctagctgactgcgggaacaaaggggttacttcacctagaaatcgctttcgagaatgggctgtgcaagattcgtccgacatggcactccgagaatatgttgccgtttacagtgacgcgatacgccaggaccctcatcgacacggttaggctgtgcatttccaattgcgatgccgccatccaagactgccttcggcctaccgcatacgatctcgacgagatatggcgctggaaccacaacctgcctcccacgtacaatttctgcatgcacgaaataatctccgaccaggctcagaagttccccgacaaggaagcaatcgcatcctgggacggcagcctgacttacagacagattgatcagtattcatcattcgtcgcgcgctcacttataggcatgggagttgggctgcatgatgtcttaccagtctgctttgagaaatcccgatggacgattgtcgcggttctggccgtaatgaaagctggcgcgacgtttgtactaatggacccgacgcttccacttgcacgattgcagaacatggcccagcaggtcggcgcgaagatgatggtttcatctcggggccagtacaacctggcgacggagatcatacccaatgcaaacgtgctcgtcgtggaggaaaacacgttcagcagtctgtccgccgaacagaacggcgagcccctgccaacggttccctcctcggccctgatgtacatgatctttacatccggcagcacaggtactccgaaaggagtcaaaatctcccacgaaacctacaccagtagcgccattccccgggccaatgcggtaggttacacggaggattcccgcgttctcgacttcgcctcctacgccttcgacgtcagcatcgacagtatgctgctgaccctgggcaacggcggctgtctgtgcattccgtccgacgaagaccgcctgaacgacatcaatggcgtgatacggcgaatgaaagtcaactatgcgggcctgacgccttcggtagcccgtattctagacgcagatgtaatatcctcgctcagtggtcttggcctcggaggagaggccgtctcagcgagagatgtcaatctctggggtcaggacaccaggattattatcggctacgggccctgcgaatgcacgattggatgcacagtgaatagtagcgcggcaacgggaagagactatatctccattggccctggtaatggggcagtaatctggatagtcgatcctaacgaccatgaatctctcgtgcctctcggcgcggtaggagagctgctggtagaaggtcccatagtagggcagggctacttgaatgatccggagaaaactgcagcagcgttcattgaagacccctcatggttggttgcgggccacgaaggttaccccggtcgtcggggccgtttgtacaagaccggtgatctcggtcgatatgacccagatgggtcaggcggtatcgtgttcgtggggcgcaaggatacccaggttaaattgcgaggacaacgggtagagctcggggagatcgaaagccaattgagagccagactgccctcggagacgactgtgatcgccgaggtgattgtaccccaaggatctgggggacaaccgacgctggtggcatttgttgctgcccagacgaccaagggacatgatcataccggactcgaggcggcagagcttcccgatgagcttcgaagggcgctgtcagaagccgatgcggagctggcaaaggtattgcctcgatacatggtgccaacagcgtatattcctgtcaaccacatccctacgctgatttcaggcaaaacagaccgcaagagactgaggcagttcggcgcaaccgttgacctgcgccagctggaccaggatgctacgaacaccgcagctcgggagctgagtgatctggagcgtcgcttgcgacaggcatggagccagacgttgaagctccaagcgtgttctattcgtctacaggacaacttcttcgctcttggtggagactccctcacagccatgaagctggtctctgtatgtcggtcccagggtctcgacctcagtgtcaccagcatgtttagcaatcctaccctgtcagccatggccagcgttgtccgtatttgcgacgtcgacgtgcaaaggacagtcccggcattctcgatgatcacatcagatatgaacagtgcctgtgtagaggcagcagagccctgcggagttggcccagcagatatcgaggacatctacccctgtacgccaacacaggagtctcttttcactttctcgctcaagtcagtcaagccctacgtggcacagagagtcctatgtatcccgtctcatatcgacctcaacgcatggaggaaggcatgggaggatgtggtcgccgcactccccatcctacggacacgagtagctcagctgcaagagcctggccttcagcaagttgtactcaagaacagcatttcctggacgcaagcctcagatctagcggaatacctagagaacgaccgaacccaaaagatgaatctcggagagagtctggcccgttacgccatcgtcgaagactcagccgacggcaagcgatacatggtttggactattcaccatgtgttgtacgatgggtggtcagagccgattatcctcaagcaagtcagcgacgccctgcaagggcagccagtcgaggtaaaggcgcagatgcgagactttgtcagattcgtgcgagactcggatgacgctgcagtgcaggaattctggcggcgcgagctgaaaggtgccgtcgggccccaattcccacgtctaccctcaagagacttcatgcctaccccggacgccctagtcgagcgtcaagtctctcttgatactagctccggatcaccgtttaccatggcgacattgattcgcggcgcctgggcgctcgtcgcgtcccaatatactggaagcgacgatatcgtgttcggcgagaccctcacaggtcgcgacatcccactacctggagtcgaaagcattgttggaccattaattgcgacggtccccatccgcgtgcgcattctccggggaagtactgtggagtcctacctccaagccgtgcagcagagtgttttggcccgtaccccgtaccagcatttgggtatgcagaacatccgcaaggttagccaagatgcgcaacacgcgtgcgagactggtacgggtctggtcattcagcctgaaccagagtatgtcggcagcgaactgggcgttgagagaggggacgtggttcttgaagcgctgcatttcaacccgtatccgctgatgctggcttgcgggattcgcaagggtggtttccgggtttgtgcaagctttgacagcagcttgattgagaccaggcagatggaacgaatgcttgcccagcttgagaccgcgtgctggcagttgagtcaaggtctttcccggaaggtcgatgagatctcgtgtctccccgaagccgagttgaaccagatctggcagtggaatcggagtccacctttgtctctggacgaaacaaccagtcggttgcgtgcgaatgcaagcacaaagcctggttcaagctatcctcccgcggttgttccttgggtttgtagtccacgaaactcatctctcctctcgccaatcggctgtgttggcgagctctggcttgaaggggcccttctttcgggcgacacagttgattcacctgcctggcttgtggctggaagctctacctgtgctggccgaactggcaaagtgcaagcaaccggcgacatggttcagttgcgagaagacggcagtttagtgtttgttggtcggaaggagaatgttgttcctgttcaagggcatgcagtggacatcacagagattgaaaggcacctcgcagaacatctgccgccaacaatccgtgccgctgctactgtagtgcggtcttcatccgaccaggagctggttatgttcattgagcaaccggctgcggaggaagcctgtattgagctcctatcggagaagcgcgaaatagtgtgcgacgcgccagacaaagccttccagaccacaatttgtgcgacaatcccgggcagtcttgctgcagtgttgaagaagttggacaaatacatgcgagactcgcttccgtcatatatggctccgtccgcttacatcgtggtagagaaactgcctaatacgatggacgatatcgaccacaatttgctcaatcagatcgcctctcaggtcactccacaaattctcaacgagcttcgcgatgggttgagcaacgcttggaccaaggctacggctccaaaccatctatcagcttcagaaagcattctgcggtctgcatgggccaaagtgcttcgtgtcgatccggagcagatcgacgtggacgacaatttcttccggcgcggtggtgattccgtacttgcaatgaagcttgtgtcgagtctccgagcgcaaggctacagtttgtccgtggctgatatcttccggcacatgcgtctcagcgacgctgccagagtgatgaaggtggatgaacgctcaacagagaagatcaactcatatcagccgttttcgatgctccgtctcccagacgttcagcagttccttgccaacattgtccgaccacagctaggggaccaacactggcccattcgagacgtgttgccggtgaccgactcccaggatatggatatccgtgcaaccattcagccgccgaggacgtcaatccagtatacaatgctgtactttgacaacagcgttgacagagagcggcttttccgttcgtgcagcgacctggttaagacgcacgagattctccgtaccgtcttcatatcacacgaatccagcttcttgcaggttgtcttgaatgagttggaaattcctgtccgcgcacacaagactgacaagcagctcgatcaatacgtcgccagcctcttcagggaggacattgaatccaacttccagttaggatgtccattcctccgacttttctacgtcgagggaaacaacggcgaatcctgcctcgtcatcggcttatcccacgcccaatacgatggcgtctccctgcccagactcctccaagacctcgacgccctatacaccggcacccagctcgccaccttctccccattctccttgtacatggcccagaccagtgaagaggcaattcaaaacaaggcagcggcctactggcgcaacctcctctccagctcctcactctcgaccctcgacggaccctcctctgatcccaccgacaaagcaatcttccacactcgcccggtcaacatccatcccctcaaggagatcaccaccgcaaacctcctcacagcagcctgggcgatggtcctcgctcgccgtctccaaaccccagacgtcaccttcggcagcgtcacctccggccgcaccctcgacattcccaacgccgaaaacttcatgggcccctgctaccagctcacccccgtccgggtccccttccatcccgactggaccgcaagcgacctgctgaacttcgtccagacacagtctgccgagtctgcggcgcatgacttcctcggttttgagaagatcgctaagctcgcggggtgggcctcaggaaggcaaggctttgattcgatcgtgcatcatcaggactgggaggatttcgatatgatgccgtttggcggagggtcttgtcgggtggacattgcgaatccgcatggggatgcggcgtatccggtcaaggctgtttcgtttgtgaaggagggagagatacatgttggtgtggtctgtagtgagagagatgtgatgtttgtagacgaggttctcggggaattggctgctgcggttgtggagttggctggtcagagtacagaggtgttgctggatagcaagttgtttagtggacagtga

SEQ ID NO: 14 - DNA, 886 bp, Aspergillus fumigatus
atgtcaacagaagcacatccaacagtacaaggatgcctcgtctcgtttccaacacctcacattctcctcctcacactcaaccgcccagagaaaaggaattgcatatccctcgccacgagcgccgagatacagcgcctatggacatggttcgacacacaacctgctctatatgtggccattattaccggcacgggcgagtccttttgtgcgggcgctgacctcaagggtacactcctcttatctctattcgatggaaacatctctattctaaggaaaacaactgacaatacatcccatagaatggaacgacctcaacgcgcgtggtatcaccaacgaaatgacagcccccgggttagccggcctccctcgccgacgaggcagcaaaccaatcattgctgccgtcaacggatactgtctcgggggcggcttcgagatggtagccaactgcgatatcgtggtggccagcgagaacgccacctttggtctccccgaagtgcaacgaggaatcgccgccgtcgcaggctcgctccctcggctggtccgcgtgctgggaaaacagcgagcagccgagatcgctctgagcgggttgagcttttctgcgtcccagctcgagcggtgggggctggtgaaccgcgtggtggagcatgaccagttattggcgacagcggtggagattgcaaccgctatctctaggaatagtccggacagtgtgcgtgtgacgatggaagggctgcattacgggtgggaaatggccagtgtagaagaggctagcagtgcactggttgaccagtggtatgcaaagctcatggctggagaaaatttccacgagggagtacgagcatttgtggagaagcgaaagccacagtggaggccgtctaagttgtga

SEQ ID NO: 15 - DNA, 1389 bp, Aspergillus fumigatus; identical to SEQ ID NO: 9

SEQ ID NO: 16 - DNA, 708 bp, Aspergillus fumigatus
atgacaatcaaggctcagcccactctgcacactgcccgcctggagctggtccccctgggccatgagcatcgcgagttcaccatgaagttggacatggaccccgaggtcatgaagatggtcgccttcggccggccatttaccgaagacgaagcaatccaggttcatacctggctgatgaactgcgcaacgtcggtgcctggcttcggaacctgggtcggctttgccgaaggcgagttcgtgggttggtgggtattggctcccgtccccacgacggagaaccccaagagcttcaggaccgatcgaacggagtatggcttccgagtctcgccgaagttctggggccaagggtacgcgaaggagggggctcgggagatggttcgctatgccttcgaggaactgggtctggccgaggtgattggcgagacgatgactatcaacatggcttcgcgggcggtgatggccgggtgcgggttgacgcacgttgagaccttcttcaacaagtacgatactccaccgccaggcattgaggagggagaggtacggtattcgatcactagggaggaatggttgcggatgcaaaagcccagcatgactcgaagtcgctggtttccggctttcgccagctggctgccccgattgctcctgtccaggctctggtcctatattttccaagggcgtaggctcgcagctggggcggccagcccctga

SEQ ID NO: 17 - protein, 235 aa, Aspergillus fumigatus
MTIKAQPTLHTARLELVPLGHEHREFTMKLDMDPEVMKMVAFGRPFTEDEAIQVHTWLMNCATSVPGFGTWVGFAEGEFVGWWVLAPVPTTENPKSFRTDRTEYGFRVSPKFWGQGYAKEGAREMVRYAFEELGLAEVIGETMTINMASRAVMAGCGLTHVETFFNKYDTPPPGIEEGEVRYSITREEWLRMQKPSMTRSRWFPAFASWLPRLLLSRLWSYIFQGRRLAAGAASP

SEQ ID NO: 18 - DNA, 18 bp, Artificial Sequence
  Description of Artificial Sequence Synthetic oligonucleotide
aactacctccaccagaag

SEQ ID NO: 19 - DNA, 18 bp, Artificial Sequence
  Description of Artificial Sequence Synthetic oligonucleotide
gaacggcaatgttgtaag

SEQ ID NO: 20 - DNA, 18 bp, Artificial Sequence
  Description of Artificial Sequence Synthetic oligonucleotide
gggacaagagcaagatgc

SEQ ID NO: 21 - DNA, 18 bp, Artificial Sequence
  Description of Artificial Sequence Synthetic oligonucleotide
cccagtagaggatgcaag

SEQ ID NO: 22 - DNA, 18 bp, Artificial Sequence
  Description of Artificial Sequence Synthetic oligonucleotide
gtgaccgatcccaagaac

SEQ ID NO: 23 - DNA, 18 bp, Artificial Sequence
  Description of Artificial Sequence Synthetic oligonucleotide
ggatgggaatgtcttgtg

SEQ ID NO: 24 - DNA, 20 bp, Artificial Sequence
  Description of Artificial Sequence Synthetic oligonucleotide
atatgttcctcgtgccgttc

SEQ ID NO: 25 - DNA, 20 bp, Artificial Sequence
  Description of Artificial Sequence Synthetic oligonucleotide
ccttaccacggaaaatggca

SEQ ID NO: 26 - DNA, 20 bp, Artificial Sequence
  Description of Artificial Sequence Synthetic oligonucleotide
tcacctgctcgtcatgcgtc

SEQ ID NO: 27 - DNA, 27 bp, Artificial Sequence
  Description of Artificial Sequence Synthetic oligonucleotide
ggagtatctagatgcgacactactctc

SEQ ID NO: 28 - DNA, 20 bp, Artificial Sequence
  Description of Artificial Sequence Synthetic oligonucleotide
caccgcttgaaacccagaat

SEQ ID NO: 29 - DNA, 27 bp, Artificial Sequence
  Description of Artificial Sequence Synthetic oligonucleotide
ggagtatctagatgcgacactactctc

SEQ ID NO: 30 - DNA, 18 bp, Artificial Sequence
  Description of Artificial Sequence Synthetic oligonucleotide
gtgggattgctgatgctg

SEQ ID NO: 31 - DNA, 29 bp, Artificial Sequence
  Description of Artificial Sequence Synthetic oligonucleotide
aagattgatatcaacacctttcccataac

SEQ ID NO: 32 - DNA, 29 bp, Artificial Sequence
  Description of Artificial Sequence Synthetic oligonucleotide
aagattgatatcaacacctttcccataac

SEQ ID NO: 33 - DNA, 18 bp, Artificial Sequence
  Description of Artificial Sequence Synthetic oligonucleotide
gtggcctgccttccctcc

SEQ ID NO: 34 - DNA, 18 bp, Artificial Sequence
  Description of Artificial Sequence Synthetic oligonucleotide
ttggcgagaggagagatg

SEQ ID NO: 35 - DNA, 18 bp, Artificial Sequence
  Description of Artificial Sequence Synthetic oligonucleotide
tacgatgggtggtcagag

SEQ ID NO: 36 - DNA, 18 bp, Artificial Sequence
  Description of Artificial Sequence Synthetic oligonucleotide
cctcatccctatctcacc

SEQ ID NO: 37 - DNA, 18 bp, Artificial Sequence
  Description of Artificial Sequence Synthetic oligonucleotide
agttttgagcgagagggg

SEQ ID NO: 38 - DNA, 18 bp, Artificial Sequence
  Description of Artificial Sequence Synthetic oligonucleotide
acaatcaaggctcagccc

SEQ ID NO: 39 - DNA, 18 bp, Artificial Sequence
  Description of Artificial Sequence Synthetic oligonucleotide
acttcgagtcatgctggg

SEQ ID NO: 40 - DNA, 28 bp, Artificial Sequence
  Description of Artificial Sequence Synthetic oligonucleotide
gcagatcgataacttagacggcctccac

SEQ ID NO: 41 - DNA, 27 bp, Artificial Sequence
  Description of Artificial Sequence Synthetic oligonucleotide
ctcggagctcctttgagtcgccatcgc

SEQ ID NO: 42 - DNA, 28 bp, Artificial Sequence
  Description of Artificial Sequence Synthetic oligonucleotide
ctggaatctagagatcggatggcgtggg

SEQ ID NO: 43 - DNA, 28 bp, Artificial Sequence
  Description of Artificial Sequence Synthetic oligonucleotide
ctgcaagcttatggggttggcactaagc

SEQ ID NO: 44 - DNA, 18 bp, Artificial Sequence
  Description of Artificial Sequence Synthetic oligonucleotide
aatgctcgtactccctcg

SEQ ID NO: 45 - DNA, 18 bp, Artificial Sequence
  Description of Artificial Sequence Synthetic oligonucleotide
ggcttggcctaatacctg

SEQ ID NO: 46 - DNA, 18 bp, Artificial Sequence
  Description of Artificial Sequence Synthetic oligonucleotide
ccatactccatccttccc

SEQ ID NO: 47 - DNA, 18 bp, Artificial Sequence
  Description of Artificial Sequence Synthetic oligonucleotide
ttctgcgggcgatttgtg

SEQ ID NO: 48 - DNA, 24 bp, Artificial Sequence
  Description of Artificial Sequence Synthetic oligonucleotide
ggaggcgccgttgtttccctcgac

SEQ ID NO: 49 - DNA, 21 bp, Artificial Sequence
  Description of Artificial Sequence Synthetic oligonucleotide
tttccgcagatgtatcgagtc

SEQ ID NO: 50 - DNA, 18 bp, Artificial Sequence
  Description of Artificial Sequence Synthetic oligonucleotide
cagaagttccccgacaag

SEQ ID NO: 51 - DNA, 18 bp, Artificial Sequence
  Description of Artificial Sequence Synthetic oligonucleotide
agtcgtttacccagaatg

SEQ ID NO: 52 - DNA, 28 bp, Artificial Sequence
  Description of Artificial Sequence Synthetic oligonucleotide
aaggatcgatggaatatgacgaacccgc

SEQ ID NO: 53 - DNA, 27 bp, Artificial Sequence
  Description of Artificial Sequence Synthetic oligonucleotide
actctcgaggcatcacccaacatcctc

SEQ ID NO: 54 - DNA, 29 bp, Artificial Sequence
  Description of Artificial Sequence Synthetic oligonucleotide
gatattttaaatacctcatggcgtgcaac

SEQ ID NO: 55 - DNA, 30 bp, Artificial Sequence
  Description of Artificial Sequence Synthetic oligonucleotide
gtgtgcggccgcgtgtacctcttgcttccc

SEQ ID NO: 56 - DNA, 18 bp, Artificial Sequence
  Description of Artificial Sequence Synthetic oligonucleotide
gcccaccaaactgtcttc

SEQ ID NO: 57 - DNA, 18 bp, Artificial Sequence
  Description of Artificial Sequence Synthetic oligonucleotide
gcgtatggagccaagaga

SEQ ID NO: 58 - DNA, 30 bp, Artificial Sequence; identical to SEQ ID NO: 55
  Description of Artificial Sequence Synthetic oligonucleotide

SEQ ID NO: 59 - DNA, 18 bp, Artificial Sequence
  Description of Artificial Sequence Synthetic oligonucleotide
gcgtatggagccaagaga

SEQ ID NO: 60 - DNA, 27 bp, Artificial Sequence
  Description of Artificial Sequence Synthetic oligonucleotide
aagatcgatcgtcgggtccattagtac

SEQ ID NO: 61 - DNA, 29 bp, Artificial Sequence
  Description of Artificial Sequence Synthetic oligonucleotide
acggcggccgctggagaagcgaaagccac

SEQ ID NO: 62 - DNA, 27 bp, Artificial Sequence
  Description of Artificial Sequence Synthetic oligonucleotide
agctttaaaaggtaattgcggtggtgc

SEQ ID NO: 63 - DNA, 27 bp, Artificial Sequence
  Description of Artificial Sequence Synthetic oligonucleotide
aggggatccaaacgagacgaggcatcc

SEQ ID NO: 64 - DNA, 18 bp, Artificial Sequence
  Description of Artificial Sequence Synthetic oligonucleotide
gacatcatgcagcccaac

SEQ ID NO: 65 - DNA, 18 bp, Artificial Sequence
  Description of Artificial Sequence Synthetic oligonucleotide
ggtgctcttcgttttgcc

SEQ ID NO: 66 - DNA, 27 bp, Artificial Sequence
  Description of Artificial Sequence Synthetic oligonucleotide
aagatcgatcgtcgggtccattagtac

SEQ ID NO: 67 - DNA, 29 bp, Artificial Sequence
  Description of Artificial Sequence Synthetic oligonucleotide
acggcggccgctggagaagcgaaagccac

SEQ ID NO: 68 - DNA, 18 bp, Artificial Sequence
  Description of Artificial Sequence Synthetic oligonucleotide
cactgtggctttcgcttc

SEQ ID NO: 69 - DNA, 18 bp, Artificial Sequence
  Description of Artificial Sequence Synthetic oligonucleotide
ctccgacctacagacaac

SEQ ID NO: 70 - DNA, 18 bp, Artificial Sequence
  Description of Artificial Sequence Synthetic oligonucleotide
gaagatgttggcgacctc

SEQ ID NO: 71 - DNA, 18 bp, Artificial Sequence
  Description of Artificial Sequence Synthetic oligonucleotide
gagagcctgacctattgc

SEQ ID NO: 72 - DNA, 21 bp, Artificial Sequence
  Description of Artificial Sequence Synthetic oligonucleotide
aagtcgaagccttacaacatt

SEQ ID NO: 73 - DNA, 21 bp, Artificial Sequence
  Description of Artificial Sequence Synthetic oligonucleotide
gucgaagccuuacaacauutt

SEQ ID NO: 74 - DNA, 21 bp, Artificial Sequence
  Description of Artificial Sequence Synthetic oligonucleotide
aauguuguaaggcuucgactt

SEQ ID NO: 75 - DNA, 21 bp, Artificial Sequence
  Description of Artificial Sequence Synthetic oligonucleotide
aacaagtccgcttccaatatc

SEQ ID NO: 76 - DNA, 21 bp, Artificial Sequence
  Description of Artificial Sequence Synthetic oligonucleotide
caaguccgcuuccaauauctt

SEQ ID NO: 77 - DNA, 21 bp, Artificial Sequence
  Description of Artificial Sequence Synthetic oligonucleotide
gauauuggaagcggacuugtt

SEQ ID NO: 78 - DNA, 21 bp, Artificial Sequence
  Description of Artificial Sequence Synthetic oligonucleotide
aagtccgcttccaatatccat

SEQ ID NO: 79 - DNA, 21 bp, Artificial Sequence
  Description of Artificial Sequence Synthetic oligonucleotide
guccgcuuccaauauccautt

SEQ ID NO: 80 - DNA, 21 bp, Artificial Sequence
  Description of Artificial Sequence Synthetic oligonucleotide
auggauauuggaagcggactt

SEQ ID NO: 81 - DNA, 21 bp, Artificial Sequence
  Description of Artificial Sequence Synthetic oligonucleotide
aaggacaagtcgaagccttac

SEQ ID NO: 82 - DNA, 21 bp, Artificial Sequence
  Description of Artificial Sequence Synthetic oligonucleotide
ggacaagucgaagccuuactt

SEQ ID NO: 83 - DNA, 21 bp, Artificial Sequence
  Description of Artificial Sequence Synthetic oligonucleotide
guaaggcuucgacuugucctt

SEQ ID NO: 84 - DNA, 21 bp, Artificial Sequence
  Description of Artificial Sequence Synthetic oligonucleotide
aacaacgctgattatgcggga

SEQ ID NO: 85 - DNA, 21 bp, Artificial Sequence
  Description of Artificial Sequence Synthetic oligonucleotide
caacgcugauuaugcgggatt

SEQ ID NO: 86 - DNA, 21 bp, Artificial Sequence
  Description of Artificial Sequence Synthetic oligonucleotide
ucccgcauaaucagcguugtt

SEQ ID NO: 87 - DNA, 21 bp, Artificial Sequence
  Description of Artificial Sequence Synthetic oligonucleotide
aaggcgcagcaaacgacgtca

SEQ ID NO: 88 - DNA, 21 bp, Artificial Sequence
  Description of Artificial Sequence Synthetic oligonucleotide
ggcgcagcaaacgacgucatt

SEQ ID NO: 89 - DNA, 21 bp, Artificial Sequence
  Description of Artificial Sequence Synthetic oligonucleotide
ugacgucguuugcugcgcctt

SEQ ID NO: 90 - DNA, 21 bp, Artificial Sequence
  Description of Artificial Sequence Synthetic oligonucleotide
aacctgcctcccacgtacaat

SEQ ID NO: 91 - DNA, 21 bp, Artificial Sequence
  Description of Artificial Sequence Synthetic oligonucleotide
ccugccucccacguacaautt

SEQ ID NO: 92 - DNA, 21 bp, Artificial Sequence
  Description of Artificial Sequence Synthetic oligonucleotide
auuguacgugggaggcaggtt

SEQ ID NO: 93 - DNA, 21 bp, Artificial Sequence
  Description of Artificial Sequence Synthetic oligonucleotide
aagggttacttcacctagaaa

SEQ ID NO: 94 - DNA, 21 bp, Artificial Sequence
  Description of Artificial Sequence Synthetic oligonucleotide
ggguuacuucaccuagaaatt

SEQ ID NO: 95 - DNA, 21 bp, Artificial Sequence
  Description of Artificial Sequence Synthetic oligonucleotide
uuucuaggugaaguaaccctt

SEQ ID NO: 96 - DNA, 21 bp, Artificial Sequence
  Description of Artificial Sequence Synthetic oligonucleotide
aaagcattgttggaccattaa

SEQ ID NO: 97 - DNA, 21 bp, Artificial Sequence
  Description of Artificial Sequence Synthetic oligonucleotide
agcauuguuggaccauuaatt

SEQ ID NO: 98 - DNA, 21 bp, Artificial Sequence
  Description of Artificial Sequence Synthetic oligonucleotide
uuaaugguccaacaaugcutt

SEQ ID NO: 99 - DNA, 21 bp, Artificial Sequence
  Description of Artificial Sequence Synthetic oligonucleotide
aagccgtgcagcagagtgttt

SEQ ID NO: 100 - DNA, 21 bp, Artificial Sequence
  Description of Artificial Sequence Synthetic oligonucleotide
gccgugcagcagaguguuutt

SEQ ID NO: 101 - DNA, 21 bp, Artificial Sequence
  Description of Artificial Sequence Synthetic oligonucleotide
aaacacucugcugcacggctt

SEQ ID NO: 102 - DNA, 21 bp, Artificial Sequence
  Description of Artificial Sequence Synthetic oligonucleotide
aacctggcgacggagatcata

SEQ ID NO: 103 - DNA, 21 bp, Artificial Sequence
  Description of Artificial Sequence Synthetic oligonucleotide
ccuggcgacggagaucauatt

SEQ ID NO: 104 - DNA, 21 bp, Artificial Sequence
  Description of Artificial Sequence Synthetic oligonucleotide
uaugaucuccgucgccaggtt

SEQ ID NO: 105 - DNA, 21 bp, Artificial Sequence
  Description of Artificial Sequence Synthetic oligonucleotide
aaacctacaccagtagcgcca

SEQ ID NO: 106 - DNA, 21 bp, Artificial Sequence
  Description of Artificial Sequence Synthetic oligonucleotide
accuacaccaguagcgccatt

SEQ ID NO: 107 - DNA, 21 bp, Artificial Sequence
  Description of Artificial Sequence Synthetic oligonucleotide
uggcgcuacugguguaggutt

SEQ ID NO: 108 - DNA, 21 bp, Artificial Sequence
  Description of Artificial Sequence Synthetic oligonucleotide
aacctgctctatatgtggcca

SEQ ID NO: 109 - DNA, 21 bp, Artificial Sequence
  Description of Artificial Sequence Synthetic oligonucleotide
ccugcucuauauguggccatt

SEQ ID NO: 110 - DNA, 21 bp, Artificial Sequence
  Description of Artificial Sequence Synthetic oligonucleotide
uggccacauauagagcaggtt

SEQ ID NO: 111 - DNA, 21 bp, Artificial Sequence
  Description of Artificial Sequence Synthetic oligonucleotide
aaccgctatctctaggaatag

SEQ ID NO: 112 - DNA, 21 bp, Artificial Sequence
  Description of Artificial Sequence Synthetic oligonucleotide
ccgcuaucucuaggaauagtt

SEQ ID NO: 113 - DNA, 21 bp, Artificial Sequence
  Description of Artificial Sequence Synthetic oligonucleotide
cuauuccuagagauagcggtt

SEQ ID NO: 114 - DNA, 21 bp, Artificial Sequence
  Description of Artificial Sequence Synthetic oligonucleotide
aagaggctagcagtgcactgg

SEQ ID NO: 115 - DNA, 21 bp, Artificial Sequence
  Description of Artificial Sequence Synthetic oligonucleotide
gaggcuagcagugcacuggtt

SEQ ID NO: 116 - DNA, 21 bp, Artificial Sequence
  Description of Artificial Sequence Synthetic oligonucleotide
ccagugcacugcuagccuctt

SEQ ID NO: 117 - DNA, 21 bp, Artificial Sequence
  Description of Artificial Sequence Synthetic oligonucleotide
aaggaatggaacgacctcaac

SEQ ID NO: 118 - DNA, 21 bp, Artificial Sequence
  Description of Artificial Sequence Synthetic oligonucleotide
ggaauggaacgaccucaactt

SEQ ID NO: 119 - DNA, 21 bp, Artificial Sequence
  Description of Artificial Sequence Synthetic oligonucleotide
guugaggucguuccauucctt

SEQ ID NO: 120 - DNA, 21 bp, Artificial Sequence
  Description of Artificial Sequence Synthetic oligonucleotide
aacgacctcaacgcgcgtggt

SEQ ID NO: 121 - DNA, 21 bp, Artificial Sequence
  Description of Artificial Sequence Synthetic oligonucleotide
cgaccucaacgcgcguggutt

SEQ ID NO: 122 - DNA, 21 bp, Artificial Sequence
  Description of Artificial Sequence Synthetic oligonucleotide
accacgcgcguugaggucgtt

SEQ ID NO: 123 - DNA, 21 bp, Artificial Sequence
  Description of Artificial Sequence Synthetic oligonucleotide
aacgaaatgacagcccccggg

SEQ ID NO: 124 - DNA, 21 bp, Artificial Sequence
  Description of Artificial Sequence Synthetic oligonucleotide
cgaaaugacagcccccgggtt

SEQ ID NO: 125 - DNA, 21 bp, Artificial Sequence
  Description of Artificial Sequence Synthetic oligonucleotide
cccgggggcugucauuucgtt

SEQ ID NO: 126 - DNA, 21 bp, Artificial Sequence
  Description of Artificial Sequence Synthetic oligonucleotide
aagcgatcggtccatgtgtat

SEQ ID NO: 127 - DNA, 21 bp, Artificial Sequence
  Description of Artificial Sequence Synthetic oligonucleotide
gcgaucgguccauguguautt

SEQ ID NO: 128 - DNA, 21 bp, Artificial Sequence
  Description of Artificial Sequence Synthetic oligonucleotide
auacacauggaccgaucgctt

SEQ ID NO: 129 - DNA, 21 bp, Artificial Sequence
  Description of Artificial Sequence Synthetic oligonucleotide
aaaccgacacactcatcctac

SEQ ID NO: 130 - DNA, 21 bp, Artificial Sequence
  Description of Artificial Sequence Synthetic oligonucleotide
accgacacacucauccuactt

SEQ ID NO: 131 - DNA, 21 bp, Artificial Sequence
  Description of Artificial Sequence Synthetic oligonucleotide
guaggaugagugugucggutt

SEQ ID NO: 132 - DNA, 21 bp, Artificial Sequence
  Description of Artificial Sequence Synthetic oligonucleotide
aactacgagttctccatgaag

SEQ ID NO: 133 - DNA, 21 bp, Artificial Sequence
  Description of Artificial Sequence Synthetic oligonucleotide
cuacgaguucuccaugaagtt

SEQ ID NO: 134 - DNA, 21 bp, Artificial Sequence
  Description of Artificial Sequence Synthetic oligonucleotide
cuucauggagaacucguagtt

SEQ ID NO: 135 - DNA, 21 bp, Artificial Sequence
  Description of Artificial Sequence Synthetic oligonucleotide
aacgaagagcacctgcagctc

SEQ ID NO: 136 - DNA, 21 bp, Artificial Sequence
  Description of Artificial Sequence Synthetic oligonucleotide
cgaagagcaccugcagcuctt

SEQ ID NO: 137 - DNA, 21 bp, Artificial Sequence
  Description of Artificial Sequence Synthetic oligonucleotide
gagcugcaggugcucuucgtt

SEQ ID NO: 138 - DNA, 21 bp, Artificial Sequence
  Description of Artificial Sequence Synthetic oligonucleotide
aagacaagcatgtcctgtgtt

SEQ ID NO: 139 - DNA, 21 bp, Artificial Sequence
  Description of Artificial Sequence Synthetic oligonucleotide
gacaagcauguccuguguutt

SEQ ID NO: 140 - DNA, 21 bp, Artificial Sequence
  Description of Artificial Sequence Synthetic oligonucleotide
aacacaggacaugcuuguctt

SEQ ID NO: 141 - DNA, 21 bp, Artificial Sequence
  Description of Artificial Sequence Synthetic oligonucleotide
aacctccttccactggactgg

SEQ ID NO: 142 - DNA, 21 bp, Artificial Sequence
  Description of Artificial Sequence Synthetic oligonucleotide
ccuccuuccacuggacuggtt

SEQ ID NO: 143 - DNA, 21 bp, Artificial Sequence
  Description of Artificial Sequence Synthetic oligonucleotide
ccaguccaguggaaggaggtt

SEQ ID NO: 144 - DNA, 21 bp, Artificial Sequence
  Description of Artificial Sequence Synthetic oligonucleotide
aactgggtctggccgaggtga

SEQ ID NO: 145 - DNA, 21 bp, Artificial Sequence
  Description of Artificial Sequence Synthetic oligonucleotide
cugggucuggccgaggugatt

SEQ ID NO: 146 - DNA, 21 bp, Artificial Sequence
  Description of Artificial Sequence Synthetic oligonucleotide
ucaccucggccagacccagtt

SEQ ID NO: 147 - DNA, 21 bp, Artificial Sequence
  Description of Artificial Sequence Synthetic oligonucleotide
aacggagtatggcttccgagt

SEQ ID NO: 148 - DNA, 21 bp, Artificial Sequence
  Description of Artificial Sequence Synthetic oligonucleotide
cggaguauggcuuccgagutt

SEQ ID NO: 149 - DNA, 21 bp, Artificial Sequence
  Description of Artificial Sequence Synthetic oligonucleotide
acucggaagccauacuccgtt

SEQ ID NO: 150 - DNA, 21 bp, Artificial Sequence
  Description of Artificial Sequence Synthetic oligonucleotide
aagtcgctggtttccggcttt

SEQ ID NO: 151 - DNA, 21 bp, Artificial Sequence
  Description of Artificial Sequence Synthetic oligonucleotide
gucgcugguuuccggcuuutt

SEQ ID NO: 152 - DNA, 21 bp, Artificial Sequence
  Description of Artificial Sequence Synthetic oligonucleotide
aaagccggaaaccagcgactt

SEQ ID NO: 153 - DNA, 21 bp, Artificial Sequence
  Description of Artificial Sequence Synthetic oligonucleotide
aagacgaagcaatccaggttc

SEQ ID NO: 154 - DNA, 21 bp, Artificial Sequence
  Description of Artificial Sequence Synthetic oligonucleotide
gacgaagcaauccagguuctt

SEQ ID NO: 155 - DNA, 21 bp, Artificial Sequence
  Description of Artificial Sequence Synthetic oligonucleotide
gaaccuggauugcuucguctt

SEQ ID NO: 156 - DNA, 21 bp, Artificial Sequence
  Description of Artificial Sequence Synthetic oligonucleotide
aacctgggtcggctttgccga

SEQ ID NO: 157 - DNA, 21 bp, Artificial Sequence
  Description of Artificial Sequence Synthetic oligonucleotide
ccugggucggcuuugccgatt

SEQ ID NO: 158 - DNA, 21 bp, Artificial Sequence
  Description of Artificial Sequence Synthetic oligonucleotide
ucggcaaagccgacccaggtt

SEQ ID NO: 159 - DNA, 21 bp, Artificial Sequence
  Description of Artificial Sequence Synthetic oligonucleotide
aaggcgagttcgtgggttggt

SEQ ID NO: 160 - DNA, 21 bp, Artificial Sequence
  Description of Artificial Sequence Synthetic oligonucleotide
ggcgaguucguggguuggutt

SEQ ID NO: 161 - DNA, 21 bp, Artificial Sequence
  Description of Artificial Sequence Synthetic oligonucleotide
accaacccacgaacucgcctt